SEQUENCE LISTING (55 sequences)

SEQ ID NO: 1 - protein, 407 aa, Arabidopsis thaliana
MMSINCFRTAKAPLQSFVAVRTNSRPRNSSNRLVSVFGRKSSSPSFVTLRVVSSMAYEKELDAAKKAASLAARLCQKVQKALLQSDVQSKSDKSPVTVADYGSQAVVSLVLEKELSSEPFSLVAEEDSGDLRKDGSQDTLERITKLVNDTLATEESFNGSTLSTDDLLRAIDCGTSEGGPNGRHWVLDPIDGTKGFLRGDQYAVALGLLEEGKVVLGVLACPNLPLASIAGNNKNKSSSDEIGCLFFATIGSGTYMQLLDSKSSPVKVQVSSVENPEEASFFESFEGAHSLHDLSSSIANKLGVKAPPVRIDSQAKYGALSRGDGAIYLRFPHKGYREKIWDHVAGAIVVTEAGGIVTDAAGKPLDFSKGKYLDLDTGIIVANEKLMPLLLKAVRDSIAEQEKASAL

SEQ ID NO: 2 - DNA, 2122 bp, Arabidopsis thaliana
gacatatatttatcttcttgaaaagcgaatgatgtctataaattgttttcgaacagcgaaggctccgcttcaatcatttgtagcagtaagaacgaattcgagacctagaaattcatcgaaccgtctcgtttctgtattcggacgcaagtcttcttctccttcatttgttactctcagagttgtttcatcgatggcttacgagaaagagcttgatgctgctaagaaagctgcttcactcgctgctcgtctctgtcaggttagggttttttcgattcaatcatgacccatagattctaaagtttgattctttaagaaacccattttgtaaatcttccaaatttcgtttaacattttgtgtttattgtgcattgcatctgtaattgggaatagattctagtgatatagtgtaatggtcctctacatacgaagctcgtgtaaatctttgatcaaaatcttatctttgtgttttgggtttgtttcagaaagttcaaaaggctttgttgcaatcagatgtgcaatcaaaatctgataaaagtccagtgaccgttgctgattatggttagtttgttatacctgtccctgattagaaaaagctcttctctttgaatgttactgagattgttaggaaatcacttaatttgatctgtcttgtgttgaatttcaggttcacaagcagttgttagtttagtcttagaaaaagagctcagttctgaacccttttcattggtggctgaagaggtgaaactgcttaataaatccttgttagatgtctcacactttacttatctttgagtttgtgtttatggactcacattgtctaaaatgatctatataggactcaggcgatctacgcaaggatggttctcaggatactctggagcgcatcacaaaactcgtgaacgacactttggctaccgaggaatcgtttaatggctctactttgtctactgatgatctacttagagccattgactgtggaacatctgaaggtggtccaaatggtcgacactgggtcttggatccaattgatggcactaaagggtacgttttaaaactaactagcctaaagtcaaatcttcttatttcagagaaaatgtaaatttgatagaatgttgagtcagatgttatgttcctgacactgagcattttcatgattttagatttctgaggggagatcaatacgcagtagcactaggattgctcgaggaagggaaagtagttttaggtgtgcttgcttgtccaaacttgccgttagcatccatagcaggaaacaacaagaacaaatcttcgtcagacgaaattggatgcctcttctttgctacaattggttcagggacatatatgcagctcctagattcaaaatcttctcctgtaaaagtgcaagtctctagtgttgagaatcctgaagaggcatcgttcttcgagtcattcgaaggagctcactctctacatgacttatccagctccattgccaatgtaaattgcttctttccttccatgtgattccagctaatagctaactaattttcctcatccatttgatcatgttctatgttgtaatatacagaaactcggtgtcaaagctccaccagtccgtattgatagccaagcaaagtatggagctttatcaagaggagatggagctatatacttacggtttcctcataaaggataccgcgaaaagatttgggaccatgtcgctggtgctatagttgttacaggtaacattaaagcttactctctatgaagctaattttatagtgtcgacatgcggatgtaaatagataaggaatgcaaggttgattcttctttttggtgcagaggcgggtggaatagtgacagatgcagcaggaaagccactggatttctcgaaagggaagtatcttgatttggacacaggcattatcgttgctaacgagaagctaatgcctctgcttttgaaagcagttcgtgactccatagctgagcaagagaaagcttcagctctctgatttgtttttttctctcgtacgttctttgtttctctgtaactgttgtttcattttctttcaccgaatttcaccagtgagaatttcttccattttcgaaaaagaaataaaaatgaaattctgttttgggctaa

SEQ ID NO: 3 - protein, 405 aa, Brassica rapa
MSSINCFRTAKAPLHTFASVSRFRYSSNRIVSVSSRKSSPSFVTLRVVSSMAYEKELDAAKKAASLAARLCQKVQKALLQSDVQSKSDKSPVTVADYGSQAVVSLVLERELTSEPFSLVAEEDSADLRKDGSEDLLERITKLVNDTLATEDLTKAIDSTLTTDDLLRAIDCGTSEGGPNGRHWVLDPIDGTKGFLRGDQYAVALGLLEEGKVVLGVLACPNLPLTSIAGNKNSSSSDEIGCLFFATIGSGTYMQPLDSKSEPVKVHVSSVENPEEASFFESFEGAHSLHDLSSSIASKLGVKAPPVRIDSQAKYGALSRGDGAIYLRFPHKGYREKIWDHVAGAIVVTEAGGIVTDAGGKPLDFSKGKYLDLDTGIIVANEKLMPLLLKAVRDSIAEQEKASSSL

SEQ ID NO: 4 - protein, 358 aa, Brassica rapa
MAYEKELDAAKKAASLAARLCQKVQKALLQSDVQSKSDKSPVTVADYGSQAVVSLLLQRELISEPFSLVAEEDSADLRKEGSQDIIERITKLVNDTLATEDPLKPIDSTLSTDDILRAIDCGTSEGGPNGRHWVLDPIDGTKGFLRGDQYAVALGLLEEGKVVLGVLACPNLPLASIAGNDNKNKNSSSSEEKGCLFYATIGSGTYMQPLDSKSEPVKVHVSSVENPEDASFFESFEGAHSLHDLSSSIANKLGVKAPPVRIDSQAKYGALSRGDGAIYLRFPHKGYREKIWDHVAGAIVVTEAGGIVTDAGGKALDFSKGKYLDLDTGIIVANEKIMPLLLKAVRDSIAEQENASSL

SEQ ID NO: 5 - protein, 350 aa, Glycine max
MPYEKELAAAKKAVTLAARLCQKVQKALLQSDVHSKSDKSPVTVADYGSQALVSFILERELPSESFSLVAEEDSGDLRKESGQDTLNRITELVNDTLASEGSNSFSTLTTDDVLVAIDSGKSEGGSVGRHWVLDPIDGTKGFVRGDQYAIALALLHEGKIVLGVLACPNLPLASIGSNQQHSSSNEVGCLFFAKVGDGTYMQALGGSTQTRVHVCDIDNPEEASFFESFEAAHSLHDLSSSIAEVKLGVKAPPVRIDSQAKYGALSRGDGAIYLRFPHRGYREKIWDHAAGSIVVTEAGGIAMDAAGNPLDFSKGKFLDVVSGIIVTNQKLMPSLLTAVKEALNEKASSL

SEQ ID NO: 6 - protein, 416 aa, Phaseolus vulgaris
MRNIFSKKKKGERERKMGTATVWWRVRGISVGVGSELRLSEARIRRRGISVSTRSSCCSSPLTLIVSSMPYEKELAAAKKAATLAARLCQKVQKALLQSDVHSKSDKSPVTVADYGSQALISFILERELPSEPFSIVAEEDSGDLRKESGQETLKRITELVNDTLASEGSNTFPSLTTDEVLAAIDNGKSEGGSVGKHWVLDPIDGTKGFVRGDQYAIALGLLHEGKVVLGVLACPNLPLASIGSNQQHSSNEVGCLFFAKVGDGTYMQALGGSTQTRVHVWDIDNPEQASLFESFEAAHSSHDLSGSIAEKLGVKAPPVRIDSQAKYGALSRGDGAIYLRFPHKGYREKIWDHAAGSIVVTEAGGIVTDAAGNPLDFSKGKFLDVDTGIIVTNQKLMPSLLRAVKEALNEKASSL

SEQ ID NO: 7 - protein, 396 aa, Cucumis sativus
MAINLLKTFPKVQTSITYKPSTSLFFTTRSSFSFPSYPPVALAVCSMSYEKELAAAKKAASLAARLCQNVQKALLQSDVQSKSDRSPVTVADYGSQALVSFVLERELPSEPFSLVAEEDSGDLRTESGQETLHRITELVNETISSEGSYGASTLTAEDVLRAIDSGKSEGGPTGRHWVLDPIDGTKGFLRGDQYAIALALLDDGKVVLGVLACPNLPLGPINSSNQHSLLGDVGCLFFSTIGAGTYMQSLTGSTLTKVSVSATENPEEASFFESYEAAHSLHDLTSSIAQNLGVKAPPVRIDSQAKYGALSRGDGAIYLRFPHKGYREKIWDHAAGCIVVTEAGGVVTDAAGNALDFSKGRYLDLYKGIIVTNQRLMPSLLKAVQEALQQTASSTL

SEQ ID NO: 8 - protein, 457 aa, Malus domestica
  misc_feature (359)..(359): Xaa can be any naturally occurring amino acid
MAINWFRAVPKLRHSEPEFIRSHAFSLSFSPTKRAFSGSGRIATALSSSSSSMSYEKELAAAKKAASLAALLCQAILSLSDFITFCLRVDFKNGNPEHKLSIESASNSEHDNQYNLSSQVDFSYWFSLLQKVQKALLQSDVHSKSDKSPVTVADYGSQAIVSLVLERELPCESFSLVAEEDSGDLRKESGQETLERIKKLVNDTLANSDFSSVSNLTTEDVLRAIDNGKSEGGSSGQHWVLDPIDGTKGFVRGDQYAIALGLLDEGKVVLGVLACPNLPLASISGNNQHSSYDKVGCLFFATVGTGTYMQPLDGSYPIKVHVSATENPEEASFFESFEAAHSLHDLSSSIAQKLGVKAXPVRIDSQAKYGALSRGDGAIYLRFPHKGYREKIWDHAAGCIVVTEAGGVVADAAGNPLDFSKGKYLDLDTGIIVTNQKLMPLLLKAVRESLEEKLSSL

SEQ ID NO: 9 - protein, 349 aa, Prunus persica
MSYEKELAAAKKAASLAALLCQKVQKALLQSDIHSKSDKSPVTVADYGSQALVSLVLERELPSESFSLVAEEDSGDLRGNSGQETLQRIRTLVNDTLANNEYSSVSNLTTDDVLRAIDNGKSEGGSRGQHWVLDPIDGTKGFLRGDQYAIALALLDEGKVVLGVLACPNLPLTSISGENQQSSQDKLGCLFFATVGTGTYMQPLDGSSLLKVHVSATENPEEASLFESFEAAHSLHDLSSTIAKKLGFKAPPVRIDSQAKYGALSRGDGAIYLRFPHKGYREKIWDHAAGCIVVTEAGGVVADAAGNPLDFSKGKYLDLETGIIVTNQKLMPLLLKAVKESLEERASSL

SEQ ID NO: 10 - protein, 339 aa, Vitis vinifera
MPLFQWQVAPHIWKVQKALLQSDVQSKSDKSPVTVADYGSQAVVSFILQRELPSESFSLVAEEDSGDLRKDGAQETLARITELVNDTLTSDGTCISRVSEEDVLTAIDSGKSEGGSNGQHWVLDPIDGTKGFLRGDQYAIALALLDEGKVVLGVLACPNLPLASIGGQDQHSLHNQIGCLFSAKIGEGTDMQPLDGSSPIKVHVSAIENPEEASFFESFEAAHSKHDLSSSIAKKLGVKAPPVRIDSQAKYGALSRGDGAIYLRFPHKGYREKIWDHAAGCIVVTEAGGEVSDAAGNPLDFSKGRYLDLETGIIVTNKKLMPSLLKAVRECLDEKPSSL

SEQ ID NO: 11 - protein, 419 aa, Unknown
  Eucalyptus grandis
MATAAVSVSSCLRAVVASSSSSSTSSCSYSYCYFYSRRHLPTTSRTPKFAKPHFVSLPSFVASRPLVVAAAAMSYDKEFAAAKKAASLAARLCQKVQKELLQSDVQSKSDRSPVTVADYGSQAIVSFVLERELSTEPLSLVAEEDSGDLRKDSGKDTLQRITKHVNETIATDGSYSASPLSPEDVLRAIDTGKSEGGSFGRHWVLDPIDGTKGFLRGDQYAVALALLDDGKVVLGVLACPNLPLASIAGNVTSENAVGCLFYAKVGSGTFMQSLDGSSPVKVHVSDIGNSEDASFFESYEKAHSMQDLTSSIARKLGVKAPPVRIDSQAKYGALSRGDGAIYMRFPHKGYREKIWDHAAGCIVVTEAGGVATDAAGQPLDFSKGRYLDLDTGIIVTNQKLMPSLLKAVRESLEEKSSSL

SEQ ID NO: 12 - protein, 419 aa, Unknown
  Eucalyptus grandis
MATAAVSVSSCLRAVVASSSSSSTSSCSYSYCYFYSRRHLPTTSRTPKFAKPHFVSLPSFVASRPLVVAAAAMSYDKEFAAAKKAASLAARLCQKVQKELLQSDVQSKSDRSPVTVADYGSQAIVSFVLERELSTEPLSLVAEEDSGDLRKDSGKDTLQRITKHVNETIATDGSYSASPLSPEDVLRAIDTGKSEGGSFGRHWVLDPIDGTKGFLRGDQYAVALALLDDGKVVLGVLACPNLPLASIAGNVTSENAVGCLFYAKVGSGTFMQSLDGSSPVKVHVSDIGNSEDASFFESYEKAHSMQDLTSSIARKLGVKAPPVRIDSQAKYGALSRGDGAIYFRFPCKGYREKIWDHAAGCIVVTEAGGVVIDAAGRPLDFSKGRHLDSDTGIIATNRKLMPSILKAVRESLEEKPTSW

SEQ ID NO: 13 - protein, 348 aa, Carica papaya
MSYDKELAAAKKAASLAARLCQKVQSALLQSDVQTKSDRSPVTVADYGSQALVSFVLERELQSEPFSLVAEEDSGDLRKDGTQEVLERITKAVNDALASDGSYSTTLSKEDVLRAIDNGKSEGGSHGRHWVLDPIDGTKGFLRGDQYAVALALLDEGKVVLGVLACPNLPLAPIGVNYQHSACDEVGCLFFAEVGSGTYMQLLSGSSPVKVQVSAIENSEEASFFESYEKAHSMHDLSSSIAKKLGVKAAPVRIDSQAKYGALSRGDGTIYLRFPHKGYREKIWDHAAGSIVVTEAGGVVTDAAGNPLDFSKGKFLDLEVGIIVTNQKLMPSLLRAVRESIEEKASSL

SEQ ID NO: 14 - protein, 387 aa, Citrus clementina
MTIISCLRTVSKPSLISQFSKPKPKTQQSCSLVVSSIVMSYDKELAAAKKAASLAARLCLKVQKALLQSDVQSKNDKSPVTVADYGSQALVSFALQKEFPSEPFSLVAEEDSKDLRQDGAQETLERITKLVNDTLASDGAYNTSTLSTEDVIRAIDGGKSEGGSHGRHWVLDPIDGTKGFVRGDQYAIALALLDEGKVVLGVLACPNLPLASIVGDNQHSSNNEVGCLFFAQVGAGTYMQSLSGSLPVKVQVTAIENSEEASFFESYEAAHSNRDLSSLIAKKLGVKAPPVRIDSQAKYGALSRGDGAIYLRFPRKGYREKIWDHAAGSIVVTEAGGVVTDAAGYPLDFSKGKHLNLQAGIIVTNQKLMPALLKAVKESLEEQASSL

SEQ ID NO: 15 - protein, 384 aa, Gossypium raimondii
MSINWLRFVSPSLKKPPLFHLCHLPPPPPSSSAVIVAMSYDKELAAAKKAASLAARLCQKVQKALLQSDVQSKNDKSPVTVADYGSQALVSFVLQQEFPDNFSLVAEEDSKDLRKDGGQEIVERITKLVNDSLTIDGSYNVTLSTEDILRAIDNGRSEGGSQGRHWVLDPIDGTKGFLRGDQYAIALALLDGGKVVLGVLACPNLPLTSLSDAGQHSPNNKVGCLFFAVVGGGTYMQPLDGSSAVKVQVSAVENPEEASFFESYEAAHSMHDLSSLIAQKLGVKAPPVRIDSQAKYGALSRGDGAIYLRLPHKGYREKIWDHAAGCIVVSEAGGVVTDAAGQPLDFSKGKYLDLDTGIIVTNQKLMPLLFNAVRKSIQEKASSL

SEQ ID NO: 16 - protein, 347 aa, Gossypium raimondii
MSYHKELAAAKKAASLAARLCQNVQKALLQSDVQSKTDKTPVTVADYGSQALVSFVLQQELRAEFSLVAEEDSKDLRKDGAQEIVERITKLVNYSLTSDGSYNVTLSTEDVLKAIDSGRSEGGSQGQHWVLDPIDGTKGFLRGDQYAIALALLDGGKVVLGVLACPNLPLTSLKDAGQHSPNNEVGCLFFAEVGGGTYMQPLDGSSAVKVQVSAVENPEEASFFESYEAAHSMHDLTSLIAQKLGVKAPPVRIDSQAKYGALSRGDGAIYLRFPHKGYREKIWDHAAGCIVVTEAGGVVTDAAGQPLDFSKGKHLDVDTGIIVTNQKLMPLLLNAVKESLKEKAPSS

SEQ ID NO: 17 - protein, 384 aa, Theobroma cacao
MSINWLRVVFPSIIKLPPPLPLRYSSPSSCSCFSSVVVAMSYDKELTAAKKAASLAARLCQKVQKSLLQSDVQSKNDRSPVTVADYGSQALVSFVLQQEFPAEFSLVAEEDSKDLRKDGAQELVDRITKLVNDSLASDESYSVTLSTEDVLKAIDSGKSEGGSQGRHWVLDPIDGTKGFLRGGQYAIALALLDRGKVVLGVLACPNLPLTPINDQHSPNNEVGCLFFAEVGGGSYMQPLDSSSTVKVQVSGIENPEEASFFESYEAAHSMHDLSSLIAQKLGVKAPPVRIDSQVKYGALSRGDGAIYMRLPHKGYREKIWDHAAGCIVVTEAGGVVMDAAGNPLDFSKGKFLDLDTGIIVTNQKLMPILIKAVRESLEEKASPL

SEQ ID NO: 18 - protein, 400 aa, Manihot esculenta
MISINGLSLSIAKPKIAFTKNPFLIPLFFSNPKTSSSSSSQVALRPVFSSMSYDKELAAAKKAASLSARLCQKVQKALLQSDVQSKSDKSPVTVADYGSQALVSFVLQKELPSEPFSLVAEEDSGDLRKDEGQQTLERITKLVNDTLASDESYATSTLSTEDVLRAIDSGKSDGGSRGRHWVLDPIDGTKGFLRGDQYAIALALLDEGKVVLGALACPNLPLASISSTSQHPSQGEVGCLFFANIGAGTYIQLLNSSSASKVQVTAIDNPEEASFFESYEAAHSLHDLSSLIAKKLGVKAPPVRIDSQAKYGALSRGDGAIYLRFPHKGYREKIWDHAAGCIVVTEAGGVVTDAAGNPLDFSKGRYLDLDTGIIVTNQKLMPSLLKAVRESLEEKKASSL

SEQ ID NO: 19 - protein, 394 aa, Populus trichocarpa
MNLVSIIAKPEITITKNPFLVSSPLLFFSNKTKSKTSIPRVLSSSMSYNKELAAAKKAVSLAARLCQKMQKAILQSDVQSKSDKSPVTVADYGSQALVSYALQRELPSELFSLVAEEDSEDLLKDGGQETLERITKLVNDILATDGSYSDSTLSTEDIVKAIDCGKSEGGSRGRHWVLDPIDGTKGFLRGDQYAIALALLDEGTVVLGVLACPNLPLPSIAGGSQHSLPGEVGCLFFSVVGGGTYMQPLDSSSAVKVQVNATDNPEEASLFESYEAAHSMHDLSSSIVKKLGVKAPPVRIDSQAKYGALSRGDGVIYLRFPHKGYREKIWDHAAGCIVVSEAGGLVTDVAGNPLDFSRGRYLDLDTGIIVTNQKLMPLLLKAVRESIEEKASSL

SEQ ID NO: 20 - protein, 349 aa, Solanum lycopersicum
MSYDKELDAAKNAASLAARLCQKVQKALLQADVQSKSDKSPVTVADYGSQAVVSVVLQKELCSASFSLVAEEDSGDLRNEEGKSTLQRIMKLVNETLASDGTYGTAPLSEEDVLAAIDSGRSEGGPSGQHWVLDPIDGTKGFLRGDQYAIALGLLDEGKVVLGVLACPNLPLSSLASHNLQDDQDKVGCLFYAQVGSGTYMQSLDGSTPIKVHVTDLDNPEEASFFESFEAAHSLHDLSSLIAKKLGVKAPPVRIDSQAKYGALSRGDGAIYLRFPHKGYREKIWDHAAGYLVVAEAGGVVSDAAGNPLDFSKGRYLDLHEGIIVTNQKLMPALLKAVKESLNEKASSL

SEQ ID NO: 21 - protein, 349 aa, Solanum tuberosum
MSYDKELDAAKNAASLAARLCQKVQKALLQADVQSKSDKSPVTVADYGSQAVVSVVLQKELGSASFSLVAEEDSGDLRNEKGKATLQRIMKLVNETLASDGTYGTAPLSEEDVLAAIDSGRSEGGPSGQHWVLDPIDGTKGFLRGDQYAIALGLLDEGKVVLGVLSCPNLPLSSLASHNLQDDQDKVGCLFYAQVGSGTYMQSLDGSTPIKVHVTDLDNPEEASFFESFEAAHSLHDLSSLIAKKLGVKAPPVRIDSQAKYGALSRGDGAIYLRFPHKGYREKIWDHAAGYLVVAEAGGVVSDAAGNPLDFSKGRYLDLHEGIIVTNQKLMPALLKAVKESLNEKASSL

SEQ ID NO: 22 - protein, 466 aa, Medicago truncatula
MSFSSTIIRPRMLLGQTTRFNNNDAFTLSHVVQPQPLVRGSCSFSLSTTRHIGFVSKFDQTCSSASPIMEDEDQKKFSFFSKSEKEDDEEYSKELDVAVRAVQMACSLCQRVQESLISKTNHQVQSKDDNSPVTVADWSVQAVVSWILSECLGSENISIVAEEDVQTLSKSNASELLDSVVKTVNDCLAEAPRFGVQKPKSPLGNSEVLDIISRCNSTGGPSGRFWVLDPVDGTLGFVRGDQYAVALALVEDGEVVLGVLGCPNYPMRKEWLSYQHRYHRIVSKLTPPTSESWNKGCVLYAKKGTGKAWMQPLLHVNKMFVWPNHAIQIFVSNIDNPALATFCEPVEKANSSHSFTAGLAHSVGLRKQPLRVYSMVKYAAIGRGDAEVFMKFARAGYKEKIWDHAAGVIIIQEAGGVVTDAGGCPLDFSKGLYLEGLDRGIIACSGASLHGKIIDAVDASWGCSSL

SEQ ID NO: 23 - protein, 354 aa, Zea mays
MSQATEVVGNPYAAELAAAKKAVALAARLCQRVQRSILHSDIQSKADKTPVTVADYGSQVLVCLVLKKELPSHSFSIIAEEDSKDLREDGAQEIIEHITTLINETIVNDGSYNMSLSKEDVLSAIDGGKSEGGPSGRHWILDPIDGTKGFIRGDQYAVALGLLDEGKVVLGVLGCPNLPLKSTNKNNSSSFGDRIGSLFFATIGCGAQVEALEGSEPQKISVCSTNNPVDASFFESFEASHSKRDLTSSIAEKLGVRAPPVRMDSQAKYGALARGDGAIFLRIPHKSYIETVWDHAAGSIIVTEAGGMVKDAAGNDLDFCKGRYLDRDRGIIATNKHLMPLVLKAVQEAMKEEQ

SEQ ID NO: 24 - protein, 354 aa, Sorghum bicolor
MSQAPGVAGSPYAAELAAAKKAVALAARLCQRVQRGILQSDIQSKADKTPVTVADYGSQVLVCLVLKKELPSHSFSIVAEEDSKDLREDGAQEILEHITTLVNETIVNDGSYNMSLSKEDVLAAIDGGKSEGGPSGRHWILDPIDGTKGFIRGDQYAVALGLLDEGKVVLGVLGCPNLPLKSTNKNNSSFSGDQIGSLFFATIGCGAQVEALEGSEPQKISVCSINNPVDASFFESFEASHSKRDLTSSIAEKLGVQAPPVRMDSQAKYGALARGDGDIFLRIPHKSYIETVWDHAAGSIVVTEAGGMVKDASGNDLDFSKGRYLDRDRGIIATNKHLMPLVLKAVQEAMKEEQ

SEQ ID NO: 25 - protein, 353 aa, Panicum virgatum
MSQLSEVAANPYAAELAAAKKAVVLAARLCQIVQQGILQSDVQSKADRTPVTVADYGSQVLVSLVLKKELPSHSFSMVAEEDSKDLRKDGAQEVLEHITSLVNETIANDGSYNMSLSKEDVLAAIDGGKSEGGPSGRHWILDPIDGTKGFIRGDQYAVALGLLDEGKVVLGVLGCPNLPLKSTNKHNGGSSRGQVGSLFFATIGCGAEVEALEGSEPQKISVCSINNPVDASFFESFEASHSKRDLTSSIAEKLGVQAPPVRMDSQAKYGALARGDGAIFLRIPHKGYIETVWDHAAGSIVVTEAGGMVKDASGNDLDFSKGRYLNRDRGIIATNKYLMPVVLKVVQEAMEEQ

SEQ ID NO: 26 - protein, 352 aa, Setaria italica
MSQHPEAAANPYAAELAAAKKAVALASRLCRRVQQGILQSDIQSKADRTPVTVADYGSQVLVSLVLKKELPSHSFSMVAEEDSEDLRKDDAQEILEHITSLVNETIANDDSYNMSLSKEDVLAALDGGKSEGGPSGRHWILDPIDGTKGFIRGDQYAVALGLLDEGKVVLGVLGCPNLPLKSTNKHNGSSSGDQVGSLFFATIGCGAEVEALEGSEPQKITICSISNPVDASFFESLEHSKRDLTSTIAEKLGVQAPPVRMDSQAKYGALAQGDGAIYLRFSQKGYIETVWDHAAGSIIVTEAGGMVKDASGNDLDFSKGRYLDRDRGIIATNRYLMPMVLKAVQEAIKEEQ

SEQ ID NO: 27 - protein, 360 aa, Oryza sativa
MSELPSPPPPAAGNQYGAEHAAAKKAVALAARLCKKVQQDLLKLDVQTKADRTPVTVADYGSQVLVSVVLKIELPSNSFSMVAEEDSEDLRKDGAQEMLGHITKLVNETIINDGSYSITLSKEDVLVAIDGGKSEGGPSGRYWILDPIDGTKGFIRGDQYAIGLALLDEGKVVLGAMACPNLPFKSIDHNGGSSGDQVGALFSATIGCGSTVESLEGSQPQKISVCSISNPVDASFFESYERKHCMRDCTSSIAEKLGIQAPPVRIDSQAKYGAVAQGDGAIYWRFPHKRSKEAVWDHAAGSIIVTEAGGLVKDASGNDLDFSKGRYLDRDAGIIATNKYLMPLVVKAAQEAMKEEGILG

SEQ ID NO: 28 - protein, 424 aa, Brachypodium distachyon
MAARVGLSHALLASSSLARNPPPRARLLPYPTLLPLPTYRRSVSASASALPSLRRRRPFAARAMSQPPEAAGSPYAAELAAAKKAVALAARLCQTVQQELVQSDVQSKADKTPVTVADYGSQILVSLVLNMEVTSGSFSMVAEEDSEDLRKEGAEEILEHITDLVNETLAEDGSFNISLSQEGILSAIDSGKSEGGPSGRHWVLDPIDGTKGFVRGGQYAIALALLDEGKVVLGVLGCPNLPLTSICNLNGNSSGDQTGVLFSATIGCGAEVQSLDGSPPQKISVCSIDNPVNASFFESYEGAHNMRDVTGSIAEKLGVQAPPVRIDSQAKYGALARGDGAIYLRFPHKGYKEKIWDHAAGSIVVTEAGGIVTDASGKDLDFSKGRCLDDLDTGIVATNKQLMPSLLKAVQEAIKEKNQAPSPL

SEQ ID NO: 29 - protein, 358 aa, Triticum urartu
MSRPAGNPSYAAALGAAKKAVALAARLCQTVQQEIVQSDIQSKADKTPVTVADYGSQVLVSVVLNMEVTSGSFSMVAEEDSEDLRKDGAEEILERITDLVNKTLAEDGSYNLLLSKEAILSAIDTGKSEGGPSGRHWVLDPIDGTKGFVRGGQYAIALALLDEGRVVLGVLGCPNLPFTSVSNLSGSSSGDQTGALFSAAIGCGAEVQSLDGSPPQKISVCTIDNPANASFFESYEGAHSMRDFTGCVAEKLGVQAPPVRIDSQAKYGALARGDGAIYWRFPHEGYRETIWDHAAGSIVVTEAGGVVKDASGNDLDFSKGRFLDRDTGIIATNKQLMPSVLKCVQEAIKEKKQASSPL

SEQ ID NO: 30 - protein, 358 aa, Aegilops tauschii
MSGSAGSPPYAAELAAAKKAVALAARLCQTVQQEILQSDIQSKADKSPVTVADYGSQVLVSLALNMEVTSGSFSMVAEEDSEDLRKDSAEEILEHITDLVNETLAEDGSYNITLSKEGILSAIDTGKSEGGPSGRHWVLDPIDGTKGFLRGGQYAIALALLDEGKVVLGVLGCPNLPLASISNLNGSSSGDQMGALFSATVGCGAEVESLEGSPPQKISVCTIDNPVNASFFESYEGAHTMHDLTGSIAEKLGVQAPPVRIDSQAKYGALARGDGAIYLRFPHKGYKEKIWDHAAGAIVVTEAGGVVTDASGNDLDFSKGRFLDVDTGIIATNKQLMPSLLKSVQEAIKEKSQAPSPL

SEQ ID NO: 31 - protein, 414 aa, Zea mays
MAARVGLLPHAILVSSRALPTPRARVPYPLPPVPSYLRHASSSASRSCRASPRFVAVRAMASGNPYAAELAAAKKAVTLAAKLCQTVQQDIMHSGVQAKADKSPVTVADYGSQILVGFSLKMDVSSGPFSLVAEEDSDELRKDGAEEILEDITDLVNETIFDDGSYNISFTKEGILSAIDDGKSEGGPSGRHWVLDPIDGTKGFLRGDQYAIALALLDEGKVVLGVLACPNLPLSSINNINGNSSGDKVGALFSATIGCGAQVESLDGSPPQKISVCSIDNPVNASFFESYESAHSMHDLTGSIAEKLGVQAPPVRIDSQAKYGALARGDGAIYLRFPHKGYREKIWDHAAGSIVVTEAGGIVTDAAGNDLDFSKGRFLDLDTGIIATNKELMPSLLKAVQEAIKETNQAASLL

SEQ ID NO: 32 - protein, 414 aa, Zea mays; identical to SEQ ID NO: 31

SEQ ID NO: 33 - protein, 412 aa, Sorghum bicolor
MAARVGLFLPHALLVPTARARLPYPPPPPIPTYRRRASSSFASRPRRASPRLLPVRAMASGNPYAAELAAAKKAVTLAARLCKTVQQDIVHSGVQAKADKSPVTVADYGSQILVSLALKMDVASGPFSLVAEEDSEELRKEGAEEILEEITDLVNETIVDDGSYNISFTKEGILSAIDDGKSEGGPSGRHWVLDPIDGTKGFLRGDQYAIALALLDEGKVVLGVLACPNLPLSSVNNLNGNSSGDQVGALFSATIGCGTEVESLYGSPPQKISVCSIDDPVNASFFESYESAHSMHDLTGSIAKKLGVQAPPVRIDSQAKYGALARGDGAIYLRFPHKGYREKIWDHAAGSIVVTEAGGIVTDAAGNDLDFSKGRFLDLDTGIIATNKNLMPSLLKAVQEAIKETNQASSLL

SEQ ID NO: 34 - protein, 424 aa, Setaria italica
MAARVGLVLPHALLASSRALTSCRARARLPNPPPTFPTTYHRSASSSAASPPRLRSRSHRASPRVLPVRSMASGNPYAAELAAAKKAVTLAARLCKTVQQDIVHSGIQSKADKSPVTVADYGSQILVSLALTMEVSGPFSLVAEEDSEELRKDGAEEILEEITDLVNETIVDDGSYNISFSKEGILSAIDDGKSEGGSSGRHWVLDPIDGTKGFLRGDQYAIALALLDEGKIVLGVLACPNLPLSSISNPNGSSSGNQVGALFSATIGCGAEVESLDGSPPQKIGVCSIDNPVNASFFESYESAHSMHDLTSSIAEKLGVLAPPVRIDSQAKYGALARGDGAIYLRFPHKGYREKIWDHAAGSIVVTEAGGIVTDAAGNDLDFSQGRFLDLDTGIIATNKQLMPSLLKAVQDAIKEKNQAASLL

SEQ ID NO: 35 - protein, 437 aa, Panicum virgatum
MDMAMAARLVPLVLPHAALLGSSAAASTRALPLAPSRAPRLPYPLPHFPTCRRPSSSSSSAAAYAARPRPRRGSPRLLAVRAMASGNPYAAELAAAKKAVTLAARLCQAVQQDIVHSGIQSKADKSPVTVADYGSQILVSLVLKMEVSSGPFSLVAEEDSEELRKDGAEEILEEITDLVNETISDSGSYNISFMKEGILSAIDDGKSKGGPTGRHWVLDPIDGTKGFVRGDQYAIALALLDEGKVVLGVLACPNLPLSSISNPNGSSSGGQVGALFSATIGCGAEVESLDGSPPQKISVCSIDNPVNASFFESYESAHSMHDLTGSIAEKLGVLAPPVRIDSQAKYGAMARGDGAIYLRFPHKGYKEKIWDHAAGSIVVTEAGGIVTDAAGNDLDFSKGRFLDLDTGIIATNKQLMPSLLKAVQDAIKEKNQAASLL

SEQ ID NO: 36 - protein, 358 aa, Oryza sativa
MSQAAGNPYAAELAAAKKAVTLAARLCQAVQKDILQSGVQSKADQSPVTVADYGSQILVSLVLKMEAPASSSFSMVAEEDSEELRKEGAEEILENITELVNETIVDDGTYSIYFSKEGILSAIDDGKSEGGPSGRHWVLDPIDGTKGFLRGDQYAIALALLDEGKVVLGVLACPNLSLGSIGNLNGGSSGDQVGALFSATIGCGAEVESLQGSPAQKISVCSIDNPVEASFFESYEGAHSLRDLTGSIAEKLGVQAPPVRIDSQAKYGALARGDGAIYLRFPHKGYREKIWDHAAGSIVVTEAGGLVTDASGNDLDFSKGRFLDLDTGIIATNKQLMPSLLKAVQDAIKEQNQAASPL

SEQ ID NO: 37 - protein, 363 aa, Triticum urartu
MSLKIIEQPELILLVKLIALTAYDMHFSPLSDATSTMIPFEELQQLFPSIPLVAEEDSAFIRSSNAADSKSNTLVESISSFFTKHVNNNGPPLTHDDVLRAIDRGGKDAVSFDSNPATYWLLDPIDGTKGFLKGEDSLYVVGLALVVDGKLAVGVMGCPNLAETTVGDTEDESIAACPGHGILMVSHVGCGTWSRPMSAEISQLTTAPDVWKRCSVDPCSVAQMARFCIVDSHTWDMMPLSAHFSSTMDESEPRDENKILLQNSCGGSLSKYLLVACGRMSVFILLARAEKLLKAWDHAVGVICVEEAGGQTCDWSGKPLDFGADLTGHRIIYPSGGVLATNGALHDKLVEMVSANYKNSNVE

SEQ ID NO: 38 - protein, 448 aa, Aegilops tauschii
MPTRGSFDKTMRLPVCEGPSLETEIMSRTRINLQDYLEKSQKLKVRASMLVGDEKILEKNDQTHVTIADFGVQALISFEEDSAFIRSSNAADSKSNTLVESISSFFTKHVNNNGPPLTHDDVLRAIDRGGKDAVSFDSNPATYWLLDPIDGTKGFLKGEDSLYVAWGSGEVLPTCAKWSWVRCGLSALHHAGVRLTSYNPSPDPPGVGASSTGSVLSFICIDTICVVCSNTLVGLALVVDGKLAVGVMGCPNLTDTTVGDTEDESIAACPGHGILMVSHVGCGTWTRPMSADQLTTAPDIWKRCSVDPCSVAHMARFCIVDSHTWDMMPLSAHFSSTMDESEPRDENKILLQNSCGGSLSKYLLVACGRMSVFILLARAEKLLKAWDHAVGVICVEEAGGQTCDWSGKPLDFGADLTGRRIIYPSGGVLATNGALHDKLVEMVSANYK

SEQ ID NO: 39 - protein, 424 aa, Aegilops tauschii
MGVPKSHVALESQSGNLKMGEAVPLKMRRLCSFHMFHNVYKRIPLWHRLTSFRPLEIISSHQYRCQEGAVLAEVKRSLLSGGRKILEKNDQSPVTIADFGVQALISFELQQLFPSIPLVAEEDSTFLKSSNPDDNSGNVLIESISSAVADKVKNSGSPLTHDDVLRAIDRGGKDAVSFDSNPATYWVLDPIDGTKGFLKGDDALYVVGLALVVNGKVAVGVMGCPNWTNGTTANKKEENLDPCPGNGILMVSHVCCGTWSRHLSPEIGQFTTAQDVWKRCFVDACSVVHMARFCIPDSQTWNMIPLSVLFDSTTDESDTRDENKILLRYACCGSLCKYIMVASGRASVFFSRARVNTQIKAWDHAVGMICVQEAGGQISDWSGEPLDLAADLTGRRILYPSGGVLVTNGALHDKLVELISANYK

SEQ ID NO: 40 - DNA, 1417 bp, Spruce
ggttctctgcgacttgtgtgtataagtctataacatcaatcaatggcgtggtcgagccgattctcatttcaatttacgaggacttctagtgccacctcatttgttttccttccaactacaaccctacctctacagatacatttccccagcgtgagtgcgaggaggagcatcaccaccaagacaacaagagcgaaaatggacatcggtgcatacgaacaagatcttgccattgcaatcaaggccgcctctctcgcagctcgtctatgccagtcggtgcaaaagagccttttacaaacagatactcaagccaagatggacagttctcctgtcactgttgcggattatggttctcaggctttagttagctttgtgcttgagagggaactacaatcgggagtgttttccatggttgcagaagaggattcaggagatttacagaagaatgatgcacaagatatggtggaacgcattacagcacttgtaaatgaaaccatttctaatgatagtgcctataatatttctccattaacaacaggagatgtacttgcagcaatagatagaggcaaatctgaaggagggccacatggtcggcactgggttttggaccccattgatgggacaaaaggatttcttagaggagaccaatatgctgtagccttgggcttattggatgaaggagaagttattttgggtgtgctggcctgcccaaatttgccctggacatcagttagcatcagtgcccgcccttcaaatgatccaattggttgccttttttctgcaagaaaagtgccgttgaaaagtctgaagatgcatctttctttgaatcatatgaggcagcacattccatgcatgacttgacagctacaatagcaaagatcctgggtgtaaaagcaccaccagttagaatagatagtcaggcaaaatatggagcaatggcaaggggtgatggagcaatatatcttcgctttcctcgtccaggctatcgtgaaaagatttgggatcatgcagctggttgcatcgtaatccaagaggctggtggtgttgtggttgatgctgctgggaaacctcttgatttctcacaggggaggtatctggatgtggaaaggggtataattgcaaccaatgccaaactgatgccattgcttcttaatgcagtgcaagctgctctgaaggaagaaggaaatgttcgaaaggctggttccttgtagcaccattcatttattgaacctaattagggatggaagttggaacttatgctgatgaaataaaacaattctatttgcacaaggtccttcctttaagagatataactgagtgtcgtggtaggtgtgtcaactctctggctctagtcctggccacatttgcagtgatagacaataagttgaaaacggaagtacattaatttcctttaattttc

SEQ ID NO: 41 - DNA, 1070 bp, Pine
caagagtgaagatggagaacagggcatacgaacaagatcttgccattgcaatcagggctgcctctctggcagctcgtctatgccagtcagtgcagaagagccttttacaaacagatactcaagccaagacggacagttctcctgtcactgttgcagattacggttctcaggctttagttagctttgtacttgaaagggaattaccctcgggattgttttccttggttgcagaagaggattcaggagatttacagaagaatgatgcacaagatatggtggaacgcattacagcacttgtaaatgaaaccatttctaatgatggtacctatgatatttctccattaacaacaggagatgtacttgcagcgatagatagaggtaaatctgaaggagggccccatggtcggcattgggttttagaccccattgatgggacaaagggatttcttaggggagaccaatatgctgtagccttaggcttattggatgaaggagaagttattttgggtgtgctggcctgcccaaatttgccctggacatcagttagcatcagtgctcgcccttcaaatgatccaattggttgtcttttttctgcaataaaaggagctggaactactgtgcaatcattggatggttctatacaacctaaaagggtttatgtaagtgccatagaaaagtctgaagaagcatctttttttgaatcatacgaggcagcacattccatgcatgacttgacggctacaatagcgaagatcctgggtgtaaaagcacccccagttagaatagatagtcaggcaaaatatggtgcaatggcaaggggtgatggagcaatatattttcgctttccttgtccaggctatcgtgaaaagatttgggatcatgcagctggttgtattgtaatccaagaaaattcatccgaattgcagaggctggtggtgttgtagttgatgctgctggaaaacctcttgatttctcacgggggaggtatctggatgtggaaaagggtataattgcaaccaatgccaaactgatgccattgctttttaatgcagtgcaagctgctctcaagg

SEQ ID NO: 42 - DNA, 1544 bp, Medicago
tggggacgcagagtttgttgggaggaacaagaacggtagtagcaataagggattgttgcgaatccataaatacaataataataatcaggaaaaaatccaccaccgtctctggtttctcattatcatcatcaacgacaagaagatcctcttcattgtattgtcgtcgttggtcaccattaacgacaatactttcttcttcttctttaatgtcttcttacgagaaggaactcgctgctgctaagaaagctgccactctcgctgctcgtctttgccagaaagtacaaaaagctcttcttcaatctgatgttcactctaaatctgacaaaactcctgtcactgttgctgattatggttcgcagatcttggtcagcttgatgcttcagagagagcttccttctgaaccattttcattagtagctgaggaggattcaggggatcttcgtaaggaaagtggccaagatacattgaagcgcattacagatcttgtcaatgatactcttgttaatgaaggatcacataacatttctgctttaacaacagatgacgtgcttaatgccattgataatggtaagtccgaaggtggttccattgggcggcactgggttttggatccgatagatggtactaaaggttttgtaagaggagaccaatatgccatagcattagctctgctagatgaagggaaagttgtattgggtgtcttggcttgtccaaatcttccgctgggaaccattggcccgaatcaaccgcattcttcttctaatgaagttgggtgtcttttctttgccaaagttggcaatggaacatatatgcaagcattggatggtactacacaaactaaggtgaatgttagtactgttgacaatccagaagaagcatcattttttgaatcttatgaagcagcacactcctcacatgacttgtctagcactattgcagtaaaactcggcgtcaaagcaccgccagtcagaattgacagccaggcaaaatacggagctctatccagaggagatggggctatatatttgcgtttccccaacaaaggataccgtgaaaaaatatgggatcatgctgctggttgtattgttgtgagtgaagctggaggtattgtctcagatgctgccggaaaccctttggacttctcaaaaggaaagttccttgatgttgatactggtattattgttacaaaccagaacttgatgccttcgcttttgagagcagttaaagaatcactcaatgagaaagcatcatccttgtaatatctgtcaagtgtatgattctactactgagcttcctcattttgttgtcagccagttatatgaaccttcctctcaattgtgctacatttcatacgcgatctgatgaagctaagcatatgactggacttcctccaacactttatttaaatcatatgcctgttctgctttttgcgtgtactgtcttcaagattcttgtaatttaagcttgagttgtaggtcctttttcttcttgcaagtacttttttcctcccaactttcttttctattaatagatatttttatttc

SEQ ID NO: 43 - DNA, 892 bp, Lotus
  misc_feature (25)..(25): n is a, c, g, or t
agaatctggaaaacaaccatcaccngttgtgatctttcatcaaggggaagaagaagaagaccagtttccttgtcttgttgttgttcatcactttcttcaatgccttacgagaaggagctcgctgctgccaagaaagcagccactctcgctgctcgtctctgtcagaaagtacaaaaggctcttctgcaatccgatgtccactcaaaatcagacaaaagtcctgtcaccgtcgctgattatggttcacaaaccttggtcagcttaatactcgagagagaacttccttctgaaccattttcattagtagctgaggaggattcaggcgatctgcgtgaggaaagtggccacgatactttgaagcggattacagaacttgtcaatgatactcttgctaatgaaggatcaaatagcttttctactttaacaacagacaatgtgcttagagccattgacaatggtaagtctgaaggtggctctgttggacggcactgggttttggatccaatagatggcactaaagggtttgtaagaggagaccaatacgctattgcattagctttgctaaatgaaggaaaagttgtattgggtgtcttggcttgcccgaatcttccactggcatccattgcctgtaatcagcagcattctacttctaatgaagttggttgtcttttctttgctaaagttggcgatggaacatttatgcaggcaatggatggttctacacagactagggtgcatgtcagtgctattgataatccagaagaggcatcattttttgagtcttttgaagcagcgcactccttacatgacttgtctagctccattgcagaaaaacttggtgttaaggcaccgccagtcagaattgacagccaagcaaaatatggagctctgt

SEQ ID NO: 44 - DNA, 1514 bp, Soybean
gaaaaagatgggtactcaaacaacactttggtgtagagcaagaactactataagtgtaggaggtgattggaagttgttcgaatccagaatccgaaaaaagggcaccaccgtgtcaacaagtagaagcagaagctgtttttgttcttcgccgctgacactaatcgtttcttcaatgccttacgagaaggaattcgccgctgcaaaaaaagcagccactctcgctgctcgtctctgcaagaaagtacagaaggctcttctgcaatccgatgtgcactcaaagtcagacaaaagtcctgtcacagtggctgattatggttcacaagcattggtcagctttatacttgagagagaacttccttctgaaccattttcattagtagctgaggaggattcaggtgatcttcgtaaggagagtggtcaggatacgctgaagcgcattacagaacttgtcaatgatacccttgctagtgaaggatcaaatagcttttctactttaacaacagatgatgtgcttgcggccattgacggtggtaaatctgaaggtggttcagttggacggcactgggttttggatccgatagatggtactaaagggtttgtaagaggagatcaatatgctatagcgttagctttactagatgaaggcaaagttgtattgggtgtcttggcttgtccaaaccttccactggcatccattggctccaatcagcagcattcttcttcaaatgaagttggttgtctcttctttgctaaagttggtgatggaacatatatgcaagcactgggcggttctacacaaactagggtgcatgtctgtgatattgataacccagaggaagcatcatttttcgaatcttttgaagcagcacactccttgcatgacttatctagctcaatcgcagaaaaacttggtgtcaaagcaccaccagtcagaattgatagccaagcaaaatatggagctttgtcaagaggagatggggctatatatttgcgtttccctcacagaggataccgtgaaaaaatatgggatcatgctgctggcagcattgttgtgactgaagctggaggtattgccatggatgctgcggggaaccctttggacttttcaaaaggaaagtttcttgatgttgtatctggtattattgttacaaaccagaaattgatgccatcacttctgacagcagttaaagaagcactcaatgagaaagcatcatccttgtgatttccatttaagcgaatgattctacaaatgagcttgctaacgttgttgtcggctcgtgttatatggggccttcccctcaattgtgctatatttcattctgatgaagttaaacaaatgatgggacttcctctgatacttcctttgtagcagcagatttgtgctctggttttttgcagctgctattttcttctccttctatatacctgtccttttctctccttttctattaataaacagagatgagagatcttattttaaacttgtttgataattaattcttctagattacttg

SEQ ID NO: 45 - DNA, 1252 bp, Arabidopsis thaliana
gacatatatttatcttcttgaaaagcgaatgatgtctataaattgttttcgaacagcgaaggctccgcttcaatcatttgtagcagtaagaacgaattcgagacctagaaattcatcgaaccgtctcgtttctgtattcggacgcaagtcttcttctccttcatttgttactctcagagttgtttcatcgatggcttacgagaaagagcttgatgctgctaagaaagctgcttcactcgctgctcgtctctgtcagaaagttcaaaaggctttgttgcaatcagatgtgcaatcaaaatctgataaaagtccagtgaccgttgctgattatggttcacaagcagttgttagtttagtcttagaaaaagagctcagttctgaacccttttcattggtggctgaagaggactcaggcgatctacgcaaggatggttctcaggatactctggagcgcatcacaaaactcgtgaacgacactttggctaccgaggaatcgtttaatggctctactttgtctactgatgatctacttagagccattgactgtggaacatctgaaggtggtccaaatggtcgacactgggtcttggatccaattgatggcactaaaggatttctgaggggagatcaatacgcagtagcactaggattgctcgaggaagggaaagtagttttaggtgtgcttgcttgtccaaacttgccgttagcatccatagcaggaaacaacaagaacaaatcttcgtcagacgaaattggatgcctcttctttgctacaattggttcagggacatatatgcagctcctagattcaaaatcttctcctgtaaaagtgcaagtctctagtgttgagaatcctgaagaggcatcgttcttcgagtcattcgaaggagctcactctctacatgacttatccagctccattgccaataaactcggtgtcaaagctccaccagtccgtattgatagccaagcaaagtatggagctttatcaagaggagatggagctatatacttacggtttcctcataaaggataccgcgaaaagatttgggaccatgtcgctggtgctatagttgttacagaggcgggtggaatagtgacagatgcagcaggaaagccactggatttctcgaaagggaagtatcttgatttggacacaggcattatcgttgctaacgagaagctaatgcctctgcttttgaaagcagttcgtgactccatagctgagcaagagaaagcttcagctctctga

SEQ ID NO: 46 - DNA, 764 bp, Oilseed
ggaacagcccaggcaccgcttcagacattcgcatcagtttcgaaattcagatattcaccgaaccctatcgtttctgtattgagaaggaagtcgtctccttcatttgttactctaagagtagtctcatccatggcttacgagaaagagcttgacgctgctaagaaagctgcttccctcacagctcgtctctgtcagaaagttcaaaaggctttgttgcaatcagatgttcaatcaaaatctgataaaagtccagtcaccgttgctgattatggttcacaagcagttgtcagcatagtcttggaaagggaactcacttctgaacccttttcattggtcgctgaagaggactcagcggatctacgcaaggatggttctgaggatattcttcagcgcatcacgaaactcgtcaacgacactttggctactgaggatctaaccaaagccattgactctactttaaccacagatgatctactcagagccatcgactgtggcacatctgaaggtggtcctaatggtcgacactgggtcttggatcctatcgatggcaccaaaggatttttgaggggagatcagtacgcggtagcactaggattactcgaggaagggaaagtagtgttaggtgtgcttgcttgtccaaacttgccattaacatccatagcaggaaacaagaactcttcttcttcagacgaaatcggatgcacttctttgctacgattggtcagggacatacatgcagcccttagactcgaaatcgaacagtc

SEQ ID NO: 47 - DNA, 1340 bp, Poplar
gcgaaatcacaatcacgaaaaacccttttcttgtttcctctccccttttatttttctctaacaaaaccaaaagcaaaacctcaattccaagagtattatcctcttcaatgtcttacaataaagaacttgctgctgccaagaaagctgtctctcttgctgcccgcctctgccagaagatgcaaaaagctatcttgcaatcagatgtccaatcaaaatcagataaaagtcccgtcactgttgctgattatggctctcaagcactagttagttatgctctgcagcgggagcttccttctgaactattctccttagtggcggaggaggattcagaagatctcctcaaggatggtggccaggaaacactagagcgaatcacaaaacttgttaacgatattctagctaccgatggatcatatagtgattctactttatccactgaagatattgtcaaggccattgactgtggaaaatctgaaggtgttctcgagggcagacactgggttctggacccaatagatggcactaaagggtttttaagaggagatcaatatgcaatagctttagcattgctagatgaagggacagtagtgttgggcgtcttggcttgtcccaatcttccgttaccttccattgctggtggctctcagcattctttgcctggcgaagttggttgccttttcttttctgtagttgggggtggaacttacatgcagccactggatagctcttcagcagtgaaggtgcaagtcaacgctactgacaatcctgaagaagcatcgctctttgaatcatatgaagcagcacactccatgcatgatctatctagttcaattgtcaaaaaacttggtgtcaaagcaccaccagttagaattgatagccaagcaaagtatggcgctctgtctagaggagatggggtcatatacttgcgatttccacataaaggttaccgtgagaaaatatgggatcatgctgctggatgcatagttgtatcagaagctgggggactggtcacagatgttgcggggaaccccttagatttttcaagaggaagatacctggatcttgacacaggcatcattgttacgaatcagaaactgatgccattacttttgaaggcagttagagaatccatagaggagaaagcttcatcattgtgattctttactgatgaatgaagtcatttcctttgctaagtttgttagcaactcaaatcagttttacgtgacttgaattttccactctatgtttgctcaatgtatgttgagcatgaaactcttgcaacttgtgaagttcaaatgtattatccgatgcataatagtagtgtcattctgctaagctac

SEQ ID NO: 48 - DNA, 1588 bp, Cotton
ggaattttcttacgaaatttttgaattgcaaatttaataaatgtctataaattggttgagatttgtttccccctctcttaaaaagccccctctttttcatctctgtcacttgcctcctcctcctccttcttcttctgctgtgattgtggcaatgtcttatgataaagaactggctgctgcaaagaaagcagcctctctcgctgctcgtctctgtcagaaagtacaaaatgctttgctgcaatccgatgttcaatcaaagaatgataaaagtcctgtaactgttgctgattatggctcacaagcactggttagttttgtgctgcagcaggaatttcctgataacttctcattagttgctgaggaggattctaaagatcttcgcaaggatggtggccaggaaatagtagagcgcattacaaaacttgtgaacgattctctaactattgatggatcatacaatgttactttatccacagaagatattctcagggccattgacaatggcagatctgaaggtggttcccaaggtcgacactgggttttggatcctatagatggtactaaaggttttctgagaggagatcaatatgcaatagcattggctttgctagatggaggaaaagttgtcctgggtgtgctggcttgtccaaatcttccactaacttccctcagtgatgctggtcagcattctccaaataataaagttggctgccttttctttgctgtagtaggtggtggaacttatatgcagccacttgatggttcttcggcagtaaaggtgcaagtaagtgctgttgaaaatcctgaagaagcatcattctttgagtcttatgaagcagcacactccatgcatgatttatctagcttgattgcccaaaaactcggcgtcaaagcaccaccggttagaattgatagccaggcgaagtatggtgctctatccagaggagatggagccatatatctgcgtcttccacacaaagggtatcgagaaaaaatatgggatcatgctgctgggtgtattgttgtgagtgaagctgggggtgtggtcacagatgctgcagggcagccattggatttttcaaagggaaagtatcttgatctggacacaggcatcattgtcaccaaccagaagttgatgccattactgtttaatgcagttagaaaatctatccaggagaaagcttcatctttgtgattcattttagaggcaggcttcatcctttcctcataacttgctctgttaagctggttgaaacttgattatttttgccttcaatgcttgaacttttatcattcttctccccaatgcttataggaagattttcatttagcatgcatgaacaagaatgggaccaataaatctcatctctacaattaagacaatgtaattgtagactagaacttgaaagcatggtcctaggccttgagcctttctctttgtcctacctggcagttgggtttaggtgtaattaattagataaaggaactgtgttgttgaatacaaacatattacattattactaaacttatttcatggtaaaacaagtgtgaagtcctctccttcccgaccgtttaccttacatggttt

SEQ ID NO: 49 - DNA, 1355 bp, Tomato
aaaatacaaaaccccacttccccttttcacttttgctcactctaagagcatctaaatcaaccttaagagctgttacaatgtcgtatgataaagaacttgatgctgccaagaatgctgcttctcttgctgctcgcctttgtcaaaaagtccaaaaagcactgttgcaagcagatgttcaatcaaagtctgacaaatctccagtgacggtggctgattatggctcacaggccgtggttagcgttgttttgcagaaagagttgtgttctgcttcattttcattagtggctgaggaggactctggagaccttcgtaatgaagagggaaaatcaacattacagcgtatcatgaagcttgtcaatgaaacacttgctagtgatggaacatatggtactgccccattatctgaagaagatgtccttgctgccattgatagtggtagatctgaagggggtccttctggtcagcactgggtgttggatcctattgatggtactaaagggtttctaaggggagaccaatatgcaattgcattgggattgctagatgaagggaaggtggttttgggcgtcttagcctgtccaaatcttccattatcttctcttgcctcccacaatctacaggatgatcaagacaaagttggttgccttttttatgcccaagttggttctggaacttatatgcagtctcttgatggctctacaccaataaaggtgcatgtaactgatttagacaaccctgaagaggcatctttttttgaatcttttgaagcagcacattctttgcatgacctatctagtttgatagcaaagaaacttggtgtaaaagcccccccagttcgaatagacagccaggcaaagtatggtgctttgtcccgtggagatggagcaatatatctgcggtttcctcataaaggctaccgcgagaagatatgggatcatgctgctggatatctcgttgttgcagaagctggaggtgttgtctcagatgctgcaggaaaccctttggacttctccaagggaagataccttgatttacacgaaggcataatcgttaccaatcaaaagctgatgcctgctctcctcaaggctgttaaagaatctttgaatgagaaagcttcatccttgtgatgatcaaaccaataacaatacgctgcagctatcttattgtagttcccagaatggtacttgcagctgctcattttcacctgttcttctccttctacacatttctgatatcactttttagctctttgattgtcatctcaactcagctttgataaacattttgttgttctctggttatgtcaactcaactttgattaaacattatatatttctcttggtatgttttttttt

SEQ ID NO: 50 - DNA, 1058 bp, Potato
  misc_feature (970)..(970): n is a, c, g, or t
gctttcacattatcatctaggactataaattccttgacacaaaaaaccctgccattgttaacaaaaaatacaaaaccccatttccccttttcactttgtctcactctaagagcatctaaatcaaccttaagagctgtttcaatgtcgtatgataaagaacttgatgctgccaagaatgctgcttctcttgctgctcgcctctgtcaaaaagtccaaaaagcactgttgcaagcagatgttcaatcaaagtctgacaaatctcctgtgacggtggctgattatggctcacaggccgtggttagcgttgttttgcagaaagagttgggttctgcttcattttcattagtggctgaggaggactctggagatcttcgtaatgaaaagggaaaagcaacattacagcgtatcatgaagcttgtcaatgaaacacttgctagtgatggaacatatggtactgccccattatctgaagaagatgtccttgctgccattgatagtggtagatctgaagggggtccttctggtcagcactgggtgttggatcctattgatggtactaaagggtttctgaggggagaccaatatgcaattgcattgggattgctagatgaagggaaggtggttttgggcgtcttatcctgtccaaatcttccattatcttctcttgcctcccacaatctacaggatgatcaagacaaagttggttgccttttttatgcccaagttggttctggaacttatatgcagtctcttgatggctctacaccaataaaggtgcatgtaactgatttagacaaccccttgaagaggcatctttttttgaaatcttttgaaagcagacacatttctttggcatgacctaatctagtttggatagcaaagaaaccttgggtggtaaaagccccgcaggtcgatatacacgccgggcaaagaggggcttcgccccggcgaaaatggcacaaataatctnggggtccccaaaaagggaccaggaaaaatatcgggaccacgggggagactcctggggcacacagcggggggtccacacaaggcgacag

SEQ ID NO: 51 - DNA, 1196 bp, Onion
caatttgcaaaagtccctcctcgtctacactgtgctaacctatcccccttaatctcgattaaatgttccccaattaattatctccctgtcttttctcccaccaaatctagttcactttcttgctcctccgtatctcaattaaaagccaagactgaatctagatctttatccatttctgccatggcttcgtacgagaaagatcttaccgctgccaagaaggctgcttcactggctgctcgtctatgccaaacggtgcagaaggcgatattgcagtcggatgtgcactcaaaagcagataagagtcctgttactgtggccgattatggttcccaagtattggtcagtcttgttttgagaaaagaacttccttttgattccttttcaatggttgctgaagaggattcaggagacttgcgaacagatgctggtcaagaaacattgaaacgtattacgaagcttgtaaatgacactctttcttctgatagtacttataatgatataattttatctgaagaagatatacttgtcgctattgatactggaaaatctgaaggaggccctcatgggcgacactgggtactggatcctatagatggcaccaaagggtttgtacgaggagatcagtatgccattgcgcttgcattgatagatgaaggaaaagtagttctcggagttctttcatgtccgaatcttcctcttaccccaattggtagttctaatacaaatcctactgaaaaccaagttggctgtcttttctctgctaaaattggttgtggaacagagaatgcaatcactagaatggttcggtgtcgtcaaaggtacatgttagcaatatcgaaaatccagccgacgcatcattctttgaatcatatgaagctgctcattctctacatgacttatctagctccatagctaagaagcttggcgtgcaagcaccgcctgtaagaattgatagccaggcaaaatatggtgctctttcacgaggagatggtgctatatatttacggtttccacataaagggtaccgtgagaaaatttgggatcatgcagctggctgcattgtcgtcacagaagctggaggggtagcaaccgatgctgctgggaatgccttagattctcgaaaggaagtatctttgattaatacaggcatattgtcaccaacagagctgatgccaaaacttttggagccgtacaat

SEQ ID NO: 52 - DNA, 2000 bp, Wheat
  misc_feature (1723)..(1723): n is a, c, g, or t
  misc_feature (1793)..(1793): n is a, c, g, or t
  misc_feature (1798)..(1798): n is a, c, g, or t
  misc_feature (1812)..(1812): n is a, c, g, or t
  misc_feature (1825)..(1825): n is a, c, g, or t
  misc_feature (1864)..(1864): n is a, c, g, or t
  misc_feature (1885)..(1885): n is a, c, g, or t
  misc_feature (1891)..(1891): n is a, c, g, or t
  misc_feature (1893)..(1893): n is a, c, g, or t
  misc_feature (1946)..(1946): n is a, c, g, or t
  misc_feature (1973)..(1973): n is a, c, g, or t
  misc_feature (1975)..(1975): n is a, c, g, or t
  misc_feature (1982)..(1982): n is a, c, g, or t
  misc_feature (1985)..(1985): n is a, c, g, or t
ctcgtgccgaattcggcacgaggcgcgtgggcctcctcctcgctccctcctccctcgcccgaaacccacctcgtgctcgcctcccttatcccctgccttcccaccaccttggccgccccacctaccgccgattcgcctcctccgcgccgctgcgctcccccctctggtggccccgccaagcgccacccctcttttccgcccgcgccatgtcagggtccgccggtagccccccgtacgccgccgagcttgcagccgccaagaaggccgtcgcccttgccgcccgcctatgccagactgtacaacaggaaattctgcaatcagatattcagtccaaggcagataaaagtcctgtgacagtagctgactatggatctcaagtattggtaagccttgcgttaaatatggaagtaacttctggttcattttctatggtggccgaagaggactcagaagacttgagaaaggatagcgctgaagaaattctggaacatattactgatcttgtaaatgaaactctcgctgaggatggttcatacaacattactttatctaaggaaggtatcctctctgcaattgatactgggaagtctgaaggaggtccatctggccgacattgggttctggatccaattgatgggactaaaggtttcttgagaggaggccaatatgcaatcgcactggcactgcttgatgagggcaaagttgttttgggcgtgttgggatgtccaaatcttcctttgacatcaataagcaacctcaatggtagctcatcaggagatcaaacgggggccctcttttcagctacaattggttgtggtgctgaagtagagtcattagagggctctccaccacaaaagattagtgtttgtaccatcgacaatccagtgaatgcctcgttctttgaatcctatgaaggagcacacacaatgcatgatttaactggctctatagcggagaaacttggtgtccaagctcctccagtcagaatagatagccaagcaaaatatggtgctctggcccgtggcgatggtgccatttacttgcgttttccacacaagggttataaggaaaagatatgggatcatgcagctggcgcaattgtcgtcacagaagctggaggtgtagtaacagatgcctcaggaaacgatctcgatttctcgaaagggagatttcttgatgttgacacaggcatcattgccacaaataagcagttgatgccatcactgctgaagtctgtccaggaggccatcaaggagaaaagccaggccccttccccattgtagaagcttttttggtttgaaccttccctgttgtagttggattgatccaaaacaatgatcgcaatgccacaccttattcacactgttctatgtgtaaatgacctttttgttgggacaaaggcaatccttgttgtatcacatttgtacatctaaggagtggtgacagccatgtcttaaataaaagaggcatctgcctgcagttataaatatcttagcatagctttgttcaacataaatgtcagcaacaactattctttgtgtgagcaacagccaatctcttgtattatttctttcagagatttttgatgtgcagtttatccccccttgttaaattacatacaattcaagctataatcaatattaactcttatggtaccaactaaatcatcattcatgggtacttgcagactcaacattgcagagctaaggctaataggactaancaggcacatcagctggatactctgcagaatgctttttgatggtatccagcagctaccgctcttgctcacnaaggntgggaagcatttgntcatacacatcantgaagagctccgcaaggccgtgttttggaacctccggcnactgaatggcttgcaggaacnttgccnanatgtcctgagtaaaccctgagtgcgcacacatcatccctgaacaattcgtccngagacggatcccgtgtccaagccaacnanaacggcngantggttaactcgagcc

SEQ ID NO: 53 - DNA, 1339 bp, Barley
  misc_feature (5)..(5): n is a, c, g, or t
  misc_feature (1143)..(1143): n is a, c, g, or t
  misc_feature (1152)..(1152): n is a, c, g, or t
  misc_feature (1220)..(1220): n is a, c, g, or t
  misc_feature (1270)..(1270): n is a, c, g, or t
caaanaatcctcactagaagggaaagacaaagttgcagcgtcagcgcgacctgcccgtgcccgccccacctaccgccgccggccgcccaatcgattggcaatgtcgcggcccgccggcaatcccccgtacgccgtcgagctcggcgccgcgaagaaggccgtctccctcgccgcccgcctatgccagacggtgcagcaggaaatcgtgcagtcagacattcaatctaaggcggataagactcctgtcaccgtagctgattatggatctcaggtattggtgagtcttgtgttaaatatggaagtaacctctggttccttttctatggtggccgaggaggattcagaagacttgagaaaggatggcgctgacgaaattctggagcgcattactgatctcgtaaacaaaactctcgctgaggatggttcatacaacattttattatctaaggaagctatcctctctgcacttgataccgggaagtccgagggtggtccatctggccgacattgggttctagatccaattgatgggactaaaggattcgtgagaggaggccaatatgccattgcactggcactgcttgatgagggcaaagttgtcttaggtgtgttgggatgtccaaatcttcctttgacatcaataagtaacctcagtgatagctcatcaagagatcaaaccggggccctcttttcagctgcaatcggttgtggtgctgaagagcagtctttggatggctctccaccacaaaagattagtgtttgtaccatcattgctacaaataaacagtcaatgccatcagtccttgaagcctatcaaggagcacacaccaagcgagaaaaaacaggccccgtagccaatgaaaaagggtgtccaacctcctccagtcactgaagttagatagccaaccaaaataatgatcgctatgctactccgtggacatggatgcacactgttaatggcgtaacccacacgagggttacagggaaacaatatgggatcatgcagctggctcaattgtcgtcacggaagctggaggtgtagttaaagatgcctcaggaaacgatctcgatttctcgaaaggaagatttcttgatcgtgacacaggcatcattgctacaaataaacagttgatgccatcagtncctgaagtnctgtccaggaggcaatcaaggagaaaaaacaggcctcttctccattgtaaaaggtttttttaaccttnccctgttactgaagttggattggtccaaaataatgatcgctatgctactnccttgttcatttattcacactgtttatgtgtaagtgaccttttgttggaaaaaatacaacccttgttat

SEQ ID NO: 54 - DNA, 1498 bp, Rice
gtggaattccggtcgcgccgtgcatctcgtacgtgacggggaggggcggcctcacccagggatgctgcaacggcgtcaaggggctgaacaacgccgcccgcaccaccgccgaccgccaggccgcctgccgctgccgcgagactccgcgccagcgccaccatgtcgcaggccgccgggaacccctacgccgctgagctcgccgccgccaagaaggccgtcaccctcgccgcccgcctctgccaggcggtgcaaaaggacattctgcagtctggtgttcagtctaaggcggatcaaagtccggtgacagttgccgattatgggtctcaaatattggtaagccttgtcttgaaaatggaagcaccagcttcttcttccttctctatggtggctgaggaggactcggaagaattgaggaaagaaggcgcagaagaaattttagaaaatatcaccgagctcgtaaacgaaactatcgtagatgatggtacatacagcatttacttctctaaggaaggtatcctctctgcaattgacgacggcaagtctgagggaggtccatctgggcaacactgggtgctagatccaattgatgggactaaaggtttcttaaggggagaccaatatgctattgccctggctctgcttgatgagggtaaagttgttttgggtgtattggcttgtcccaacctttctttgggatcaataggcaaccttaatggtggctcctcgggagatcaagttggtgctctcttttctgctactattggttgtggagctgaagtagagtctttacagggctctccagcacaaaagattagtgtctgttccatcgacaatccagtcgaagcttcattctttgagtcctacgaaggggcacactccttgcgtgatttaacaggctccattgcggagaaacttggtgtccaagctcctccagttagaattgatagccaagcaaaatacggtgccctagcccgaggtgacggtgccatttacttgcgttttccacacaaaggttacagagagaagatctgggatcatgcagctgggtcaatcgtcgtgacagaagctggaggtctggtgacagatgcatcaggaaacgatttggatttctccaaagggagatttcttgatctcgacacagggatcatcgcgacgaacaagcagctgatgccttcactcctgaaggctgtgcaagatgccatcaaggagcaaaaccaggctgcttccccgttgtagtagctgtctcaatccacaatccacaaatatcataatgtttccatataataataattgcagtgctgcttccccatcaacattactttaaatgtgtgtacagcaccactttgttgttgttgctgctgcaaattcagtatcttattgttgcacatttctgcatctaattggtgggtgagctggcaccacatgaatgttctttcatctggattcagaagaaataattaaattggcctgattctccctaactcggggcaatattgct

SEQ ID NO: 55 - DNA, 1544 bp, Maize
cgcacatggctgcgcgcgtgggcctgctcccgcacgcgatcctcgtctcctcccgagctctacccacgccccgagctcgcgtcccttatcctcttccacccgtcccttcctacctccgccacgcctcctcctccgcctcccgctcctgccgcgcgtcgccgcgttttgtcgcagtccgcgccatggcttcggggaacccctacgccgccgagctcgccgccgccaagaaggccgtcaccctcgccgccaaactctgccagacagttcaacaggatattatgcattctggcgttcaggctaaggcagacaaaagtcctgtcacagtggctgattatggatctcaaatattggtcggtttttccttaaagatggatgtatcatctggaccattttcattggtggctgaagaggactcagatgaattgagaaaagatggcgctgaagaaattttggaagatattactgaccttgtcaatgaaaccatctttgatgatggttcatataacatttcgtttacaaaggaaggtatactctctgcaattgatgatgggaagtctgagggaggtccatctggacgacattgggtgcttgatccgattgacggtactaaaggtttcttgaggggcgaccaatatgccattgcattggctctgcttgatgaaggtaaagttgttttgggtgtattggcttgtccaaatcttccattgtcatcaataaacaacatcaatggtaactcttcgggagataaagttggtgccctgttttctgctacaattggttgtggggctcaagtagagtccttagatggctctccaccacaaaagattagtgtttgctccatcgacaatcctgtcaatgcatcattttttgaatcctatgaaagtgcacactccatgcatgatttgactggctctattgcagagaaacttggtgtccaagctcctccagttagaatcgacagccaagcaaaatatggtgctctggcccgaggtgatggcgccatttacttgcgctttccacacaaaggttatagagagaaaatatgggaccatgcagcaggatcaattgtcgtgacagaagctggaggcatagtaacagatgctgcgggaaacgacttggatttctccaaagggagatttctggatctcgacacaggcatcatcgcaaccaacaaggagttgatgccgtcgctcctgaaagctgtccaagaggctattaaagagacgaaccaggctgcctccctcttatagctgttgtagtggtcgacaaaaaagtagttgcaaaattttcgtactggcaaacaggtgtgcatcatttctccgtggtattacagagtctagcaaatttatagcatcaacctgattggtgatcagacgtccattcgttttactgaatcaccttcttggtgcagcattgctagaagtggacaacatgaataatttgagtgagtccatagttgcctgaactcagctttggcatttgaactggttatagaaattgtgaaataattaatggatatttggaagacaaacttgaacttcaag

(19) United States
(12) Patent Application Publication
(10) Pub. No.: US 2018/0355369 A1
(43) Pub. Date: Dec. 13, 2018

(54) Method for Increasing Plant Stress Tolerance and Seed Dormancy

(71) Applicant: The Australian National University, Acton (AU)

(72) Inventors: Barry James Pogson, Aranda (AU); Gonzalo Martin Estavillo, Bonner (AU); Kai Xun Chan, Braddon (AU); Su Yin Phua, Braddon (AU); Peter Alexander Crisp, Ainslie, Australian Capital Territory (AU)

(21) Appl. No.: 15/741,084

(22) PCT Filed: Jun. 30, 2016
(86) PCT No.: PCT/AU2016/000234

§ 371 Date: Dec. 29, 2017

(30) Foreign Application Priority Data
Jul. 2, 2015 (AU) 2015902590
Jun. 17, 2016 (AU) 2016902361

(51) Int. Cl. C12N 15/82; C12N 9/16

(52) U.S. Cl. CPC C12N 15/8271; C12N 9/16; C12N 15/8273; C12N 15/8261; C12N 15/8269; C12Y 301/03007

(57) ABSTRACT

The present invention provides a method for increasing the levels of 3′-phosphoadenosine-5′-phosphate (PAP) or a derivative or analogue thereof in cells of said plant compared to an untreated or wild-type plant grown under the same stress conditions. The methods may comprise administration to the plant and/or the soil it is growing in of: PAP or a derivative or analogue thereof, a substance that enhances or promotes synthesis or accumulation of PAP or said derivative or analogue thereof; or a substance that inhibits or compromises an activity metabolising or removing PAP or said derivative or analogue thereof. Alternatively, the methods may comprise genetic modification of cells of said plant which modifications allow for increased accumulation of PAP or a derivative or analogue thereof in cells of said plant (or in seeds thereof) at least under stress conditions compared to a wild-type plant. The methods find particular application in increasing the tolerance of plants to abiotic stress conditions, which may be selected from increased salinity, increased sodium levels, drought, light stress and pH stress. The present invention also provides a method for extending the dormancy of a seed, as compared to an untreated seed, said method comprising accumulating in cells within said seed an increased level of PAP or a derivative or analogue thereof compared to an untreated or wild-type seed.

This application is a national stage application under 35 U.S.C. § 371 of International Application No. PCT/AU2016/000234, filed Jun. 30, 2016, which claims the benefit of Australian Provisional Application No. 2016902361, filed Jun. 17, 2016, and Australian Provisional Application No. 2015902590, filed Jul. 2, 2015, the disclosures of each of which are explicitly incorporated herein by reference.

### FIELD OF THE INVENTION

The present invention relates to methods and materials for increasing stress tolerance in plants, and continued growth under stress conditions as compared to untreated or wild-type plants.

### BACKGROUND TO THE INVENTION

The biggest limiting factor in crop yields across the world is abiotic stress, causing an average loss of more than 50% of potential yield (Boyer, J. S. (1982), “Plant Productivity and Environment” Science 218(4571): 443-448). Abiotic stresses include temperature extremes, salinity, acidic soils, high light intensities, drought and combinations thereof.

As plants are sessile and cannot escape these stress conditions, they respond by changing their protein and metabolite composition, morphology and physiology. These changes allow the plant to limit damage by adapting to the stress conditions and also to repair damage caused by the stress.

These changes are mediated by processes that sense the stress and/or its effects on the plant and activate multiple, complex signalling pathways. Different pathways are activated depending on the type of stress conditions experienced by the plant but often there is overlap and interaction between pathways. This overlap can lead to cross-tolerance, that is tolerance to multiple types of stress despite exposure to only one stress. This is important as stresses rarely occur in isolation. For example cold stress will also cause high light stress as the cold causes the metabolism of the plant to slow yet it is still able to harvest as much light energy as before. Likewise drought can cause heat stress as the stomata close to conserve water but as a result lose the cooling effect of transpiration, resulting in heat stress. Furthermore in nature the stress conditions rarely exist in isolation. In Australia it would be conceivable that a crop would experience drought and high light stress at the same time.

Although some components of stress signalling pathways have been studied, due to the complexity of stress response pathways the position of these components in the pathways and their interactions with other components is poorly understood and existing methods for improving the stress resistance of plants are accordingly limited.

Thus, there is a need for new methods for producing plants with increased stress tolerance.

### SUMMARY OF THE INVENTION

The present investigations have surprisingly shown that the molecule 3′-phosphoadenosine-5′-phosphate (PAP), or derivatives or analogues thereof, is a major component of the plant cell stress signaling pathway, and can mediate plant physiological responses to external stresses.

Thus, according to an aspect of the invention, there is provided a method for increasing stress tolerance of a plant, said method comprising increasing the levels of 3′-phosphoadenosine-5′-phosphate (PAP) or a derivative or analogue thereof in cells of said plant compared to an untreated or wild-type plant grown under the same stress conditions.

The levels of PAP or a derivative or analogue thereof may be increased in the nucleus of said cells.

The method may comprise administering to the plant a substance that: (i) enhances or promotes synthesis or accumulation of PAP or said derivative or analogue thereof; or (ii) inhibits or reduces an activity metabolising or removing PAP or said derivative or analogue thereof, or (iii) mimics the function of PAP

Alternatively, the method may comprise:

(i) introducing a mutation in an endogenous nucleotide sequence;

(ii) replacing an endogenous nucleotide sequence with an exogenous nucleotide sequence; or

(iii) inserting an exogenous nucleotide sequence;

in cells of said plant, wherein the resulting reduced activity, replaced activity or increased activity in said cells associated with said mutation or exogenous nucleotide sequence results in increased accumulation of PAP or said derivative or analogue thereof under at least stress conditions compared to a wild-type plant.

According to certain methods of the invention, the plants yield a greater amount of above-ground plant matter than an untreated or wild-type plant grown under the same conditions. According to other embodiments, the plants grow faster than an untreated or wild-type plant grown under the same conditions. According to other embodiments, the plants develop faster than an untreated or wild-type plant grown under the same conditions. According to another embodiment, the plants survive longer than an untreated or wild-type plant grown under the same conditions. According to another embodiment, the plants are photosynthetically viable for longer than an untreated or wild-type plant grown under the same conditions. According to another embodiment, the plants retain more water than an untreated or wild-type plant grown under the same conditions. According to another embodiment, the plants prevent loss of above-ground matter compared to an untreated or wild-type plant grown under the same conditions. In these embodiments, the conditions may comprise stress conditions and, according to a further embodiment, the stress conditions are abiotic and may further comprise stresses selected from the group comprising increased salinity, drought, nitrogen limitation and pH stress.

Plants obtained by the methods outlined above, and plant parts (including leaves, stems, roots, tubers, flowers, fruit, seeds and parts thereof) are also provided. According to an embodiment, the plants yield a greater amount of above-ground plant matter than an untreated or wild-type plant grown under the same conditions. According to another embodiment, the plants grow faster than an untreated or wild-type plant grown under the same conditions. According to another embodiment, the plants develop faster than an untreated or wild-type plant grown under the same conditions. In these embodiments, the conditions may comprise stress conditions and, according to a further embodiment, the stress conditions are abiotic and may further comprise stresses selected from the group comprising increased salinity or sodium, drought stress, light stress and pH stress.

According to another aspect of the invention, there is provided a method for extending the dormancy of a seed, as compared to an untreated seed, said method comprising accumulating in cells within said seed an increased level of PAP or a derivative or analogue thereof compared to an untreated or wild-type seed.

### BRIEF DESCRIPTION OF THE DRAWINGS

FIG. 1—Shows the ability of the sal1 mutant alx8 to tolerate light stress and drought conditions as compared to the parental wild-type Col-0, as reported in Rossel J. B. et al (2007), Plant Cell 19(12):4091-4110 and in Wilson P. B. et al (2009), Plant Journal 58(2): 299-317. A) an Arabidopsis thaliana alx8 plant grown under mild, moist and low light conditions; B) shows accumulation of reactive oxygen species (ROS; as represented by H₂O₂), as visualized with the ROS stain 3′,3′-diaminobenzinidine (DAB) in wild-type (Wt) and the sal1 mutant alx8 under low light (LL) and high light (HL) conditions; C) shows the result of subjecting the wild-type (Wt) and the sal1 mutant alx8 to drought conditions (well-watered—WW; water-stressed—WS)—the mutant survives 50% longer with less damage.

FIG. 2—provides a summary of findings relating to the enzyme SAL1 as discovered through the sal1 mutant alx8 (Estavillo G. M. et al (2011), Plant Cell 23(11): 3992-4012). A) the role of SAL1 in sulfur assimilation reactions and PAP catabolism; B) identification of the in vivo substrate of SAL1 (PAP), and the discovery of PAP accumulation with no changes in PAPS, APS or inositols (C)) in the sal1 mutant.

FIG. 3—A) shows that PAP level increase several fold during drought stress, in both wild-type plants (Col-0) and, even more so, in a sal1 mutant (alx8); B) shows that wild-type plants (Col-0) do not withstand drought conditions as well as sal1 mutants (alx8).

FIG. 4—A) shows a possible schematic of PAP-mediated intracellular stress signaling; B) provides a illustrative example of the fate of well watered (WW) plants with wild-type PAP signalling and with increased PAP signalling (through addition of PAP or derivatives or analogues thereof, or through genetic manipulation) under drought conditions.

FIG. 5—A)—PAP and ABA quantification from 5-week old plants subjected to progressive drought between days 0 (RWC=0.82±0.06), 4 (RWC=0.78±0.02) and 6 (RWC=0.38±0.05). Data are means±SD, n=4 and n=3 for ABA and PAP samples per time point, respectively; B)—Stomatal aperture, calculated using measurements of pore width and length, in leaf peels of wild type plants treated with either 100 μM PAP or 100 μM ABA over a period of 1 hr. Representative pictures are shown in (C). Graph values are means, expressed as a percentage compared to time 0, of at least 15 stomata, ±SEM. Rates of closure were compared by modelling the closure between 10-25 minutes (log-transformed data), significant difference groups (p<0.05) are denoted by #, *. Final level of closure was also considered by ANOVA across the final 30 minutes; significant difference (p<0.05) denoted (a, b, c); D) shows stomatal aperture in leaf epidermal peels of 3-week old barley plants in measuring buffer (control) for 10 min before adding 100 μM ABA or 100 μM PAP for another 50 min. Values are means±SE (n=17-20 stomata of 4 plants); E) Stomatal aperture as in (B) but treated with either 100 μM PAP or 1 mM ATP alone or in combination, in measuring buffer. The control treatment for both (C) and (D) was 1 hr of measuring buffer. Values are means of at least 8 stomata±SEM. (F) Thermography of 35-day old wild type leaves petiole fed with 250 μL of different combinations of 20 μM ABA, 100 mM LiCl, 1 mM PAP and 10 mM ATP in infiltration buffer or buffer alone (control). Each data point is the average of 3 different leaves from three individual plants per genotype. Leaves in solution were returned to growth chamber and leaf temperature allowed to stabilize for 20 min, prior to continuous measurements till 60 min. Significant difference groups (2-way ANOVA, Tukey's HSD post-hoc test, adj p<0.05) are shown as a, b, c, d.

FIG. 6—shows that petiole feeding of PAP for 1 h results in accumulation of PAP in leaves. Levels were significantly enhanced by co-application with LiCl, an inhibitor of the PAP catabolic enzyme SAL1, or with ATP, which outcompetes PAP for transport into plastids where PAP is degraded. ATP also allows PAP to be localized to its sites of action, the nucleus/cytoplasm. Results averaged from three individual plants

FIG. 7—One possible model of ABA and PAP-mediated stomata regulation. ABA binds to PYR/PYL/RCAR receptors and triggers stomatal closure primarily via ABA-receptor complexes that bind to protein phosphatase 2Cs such as ABI1, releasing their inhibition of SNF1-related protein kinase 2 (SnRK2) OST1 and promoting stomatal closure via phosphorylation of downstream proteins, examples of which are shown (see (5) for a more detailed model and references). ABI5 and ABFs represent transcription factors target of OST that regulate ABA-responsive genes; SLAC1 and KAT1 are anion and cation channels that mediate chloride efflux and potassium influx respectively, to modulate osmotic potential of the guard cells; and RboH is a NADPH oxidase that promotes the ROS burst. PAP can influence these processes (Gene Expression, ROS, ion fluxes and osmotic potential) to close stomata.

FIG. 8—A shows representative photos of two plants per genotype subjected to 10 d drought, where PAP accumulation due to the alx8 mutation is able to rescue drought tolerance in ABA-insensitive mutants abi1-1 and ost1-2, which normally cannot close stomata; B) shows representative micrograph of stomata from ost1-2 and ost1 1-2 alx8 leaf peels treated 50 μM ABA for 2 h.

FIG. 9—Hierarchical clustering of transcripts known to respond to ABA in guard cells. Clusters showing co-expression in WT and ost1 alx8 are shown (I). The ABA-insensitive ost1-2 does not respond transcriptionally to ABA like WT does. High PAP levels due to the alx8 mutation restores part of the transcriptional response to ABA to WT responses in the ost1 alx8 mutant.

FIG. 10—shows an alignment of SAL1 amino acid sequences across dicots, showing the highly conserved nature of cysteine residues in SAL1 across dicots. Arabidopsis (SEQ ID NO:1); Brassica rapa A (SEQ ID NO:3); Brassica rapa B (SEQ ID NO:4); Glycine max (SEQ ID NO:5); Phaseolus vulgaris (SEQ ID NO:6); Cucumis sativus (SEQ ID NO:7); Malus domestica (SEQ ID NO:8); Prunus persica (SEQ ID NO:9); Vitis vinifera (SEQ ID NO:10); Eucalyptus grandis A (SEQ ID NO:11); Eucalyptus grandis B (SEQ ID NO:12); Carica papaya (SEQ ID NO:13); Citrus clementina (SEQ ID NO:14); Gossypium raimondii A (SEQ ID NO:15); Gossypium raimondii B (SEQ ID NO:16); Theobroma cacao (SEQ ID NO:17); Manihot esculenta (SEQ ID NO:18); Populus trichocarpa (SEQ ID NO:19); Solanum lycopersicum (SEQ ID NO:20); Solanum tuberosum (SEQ ID NO:21); Medicago truncatula (SEQ ID NO:22).

FIG. 11—shows an alignment of SAL1 amino acid sequences across monocots, showing the highly conserved nature of cysteine residues in SAL1 across monocots. Zea mays C (SEQ ID NO:23); Sorghum bicolor B (SEQ ID NO:24); Panicum virgatum B (SEQ ID NO:25); Setaria italica B (SEQ ID NO:26); Oryza sativa B (SEQ ID NO:27); Brachypodium distachyon (SEQ ID NO:28); Triticum urartu B (SEQ ID NO:29); Aegilops tauschii C (SEQ ID NO:30); Zea mays A (SEQ ID NO:31); Zea mays B (SEQ ID NO:32); Sorghum bicolor A (SEQ ID NO:33); Setaria italica A (SEQ ID NO:34); Panicum virgatum A (SEQ ID NO:35); Oryza sativa A (SEQ ID NO:36); Triticum urartu A (SEQ ID NO:37); Aegilops tauschii A (SEQ ID NO:38); Aegilops tauschii B (SEQ ID NO:39).

FIG. 12—Sequence alignment of a range of plant nucleotide sequences encoding SAL1 homologues. Sequences were aligned using ClustalW at the EMBL-EBI website. “*” means that the nucleotides in that column are identical in all sequences in the alignment. Codons encoding putative cysteine residues corresponding with cysteines 173, 221 and 244 (119, 167 and 190 in the mature protein) of SAL1 are highlighted. Spruce (SEQ ID NO:40); Pine (SEQ ID NO:41); Medicago (SEQ ID NO:42); Lotus (SEQ ID NO:43); Soybean (SEQ ID NO:44); Arabidopsis thaliana SAL1 (SEQ ID NO:45); Oilseed (SEQ ID NO:46); Poplar (SEQ ID NO:47); Cotton (SEQ ID NO:48); Tomato (SEQ ID NO:49); Potato (SEQ ID NO:50); Onion (SEQ ID NO:51); Wheat (SEQ ID NO:52); Barley (SEQ ID NO:53); rice (SEQ ID NO:54); Maize (SEQ ID NO:55).

FIG. 13—shows the amino acid sequence for SAL1 (TAIR Accession: AASequence: 4010745380; Name: AT5G63980.1; Length: 407aa; Date last modified: 2007-08-16). The cysteines at positions 173, 221 and 244 (119, 167 and 190 in the mature protein) are highlighted.

FIG. 14—shows the SAL1 genomic sequence (TAIR Accession Sequence: 4010730406; Name: AT5G63980.1; Sequence Length (bp): 2122; Date last modified: 2007-04-17). This genomic sequence is an updated version of previous TAIR Accession No. 2160829, and which locates the start codon 162 nucleotides upstream of the presumed start codon in TAIR accession No. 2160829. The location of the codons encoding cysteines 173, 221 and 244 (119, 167 and 190 in the mature protein), the start codon (atg) and the stop codon (tga) are highlighted.

FIG. 15—In vivo AtSAL1 activity is down-regulated by oxidative stress with no change in protein abundance (WW: Well-watered, MD: Mid-Drought, LD: Late-Drought, HL: High-light, MV: Methyl Viologen, H₂O₂: Hydrogen peroxide).

FIG. 16—Down-regulation of AtSAL1 activity and concomitant PAP accumulation correlates with formation of the C167-C190 (mature protein numbering; labeled as C221-C224 in the figure) intramolecular disulfide (black arrows) in endogenous AtSAL1 during drought stress.

FIG. 17—shows redox-mediated regulation of SAL1: A) Activity of SAL1 in native protein extracts was significantly lowered in drought-stressed Arabidopsis leaf samples compared to well-watered (WW) extracts; B) Activity of the SAL1 enzyme is redox-responsive. Monitoring the fraction of active SAL1 enzyme along an oxidation gradient yielded a redox midpoint potential (Eₘ) of −329±2 mV, which is within the range of the physiologically relevant redox state in plants.

FIG. 18—Structural elucidation of AtSAL1 reveals a dimerization interface, and three redox-sensitive cysteine residues. Left inset shows a view of the 2:Fo:-:Fc: map (contoured at 1.0 σ) centered on C119 (labeled as C173 for its position in full length protein) which is located at the interface between chain A (orange sticks) and chain B (green sticks). Right inset shows view of the 2:Fo:-:Fc: map (contoured at 1.0 σ) centered on C167 and C190 (labeled as C221 and C224 for their respective positions in full length protein) capable of an intramolecular disulfide. Redox-reactive thiol groups are indicated as spheres in yellow.

FIG. 19—A) shows formation of the C167-C190 disulfide (mature protein numbering) in endogenous SAL1 protein in leaves during drought stress. Oxidized AtSAL1 proteins migrate at different rates to reduced AtSAL1 protein and the identity of the lower band (black arrows) was determined in B, where Cys-Cys disulfide pairs observed in WT AtSAL1 was compared to cysteine to alanine substitution mutants of AtSAL1 under oxidation. The different Cys-Cys disulfide pairs were identified by cross-comparison to cysteine mutants: AtSAL1 containing a C167-C190 intramolecular disulfide migrates closest to reduced AtSAL1 (black arrows). The oxidized form is absent in single, double and quadruple AtSAL1 mutants lacking either or both of C167 and C190; C) shows that the formation of the C167-C190 disulfide in AtSAL1 by oxidation is rapidly reversed by returning the redox state to reducing conditions. Vertical dashed lines indicate splicing of the gel to show these three samples side-by-side; this did not alter the interpretation of the result and all samples were run on the same gel; D) shows that the oxidation of redox-sensitive cysteine residues in AtSAL1 significantly decreases its activity. Mutagenizing cysteines in AtSAL1 to alanine abrogates the effect of oxidation on enzyme kinetics.

FIG. 20—A) shows that dimerization has a significant impact on AtSAL1 kinetics under both reducing and oxidizing conditions. Note that under oxidation with DTTox, the kinetics of monomeric AtSAL1 is unchanged whereas dimeric AtSAL1 is redox-sensitive (see FIG. 3b); B) shows that the monomer-dimer equilibrium can be shifted by formation of an intermolecular disulfide under oxidizing conditions which results in an increase in dimer abundance, or reduction of the disulfide by DTT which can dissociate the dimer; C) shows that the monomer-dimer equilibrium of AtSAL1 in vivo is shifted in favor of the dimer during oxidative stress, suggesting formation of the C119-C119 intermolecular disulfide to stabilize the dimer.

FIG. 21—Molecular Dynamics (MD) simulations suggest that C167-C190 (mature protein numbering) disulfide formation decreases flexibility of SAL1, as indicated by the decrease in root mean square fluctuation (RMSF) of backbone atoms in reduced (white circles) compared to oxidized (black squares) SAL1.

FIG. 22—A) shows the effect of oxidation on enzyme kinetics of the rice Oryza sativa SAL homolog OsSAL1; B) shows redox titration on Oryza sativa SAL1 showing that the protein is redox sensitive and has a redox midpoint potential (Eₘ) in the physiologically-relevant range.

FIG. 23—Provides a comparison between redox-sensitive cysteine residues detected in structures of the AtSAL1 homolog in rice (OsSAL1). Unlike AtSAL1 which contains both surface-exposed and intramolecular disulfide cysteines, OsSAL1 does not contain intramolecular disulfides. Surface exposed thiol groups of cysteines are indicated.

FIG. 24—A) shows germination rates (radicle emergence) of wild type and fry1-6 (T-DNA sal1 allele) of non-stratified seeds. Values show averages of five plates containing at least 70 seeds per plate; B) shows germination rates of wild type seeds in the presence of 1 μM ABA alone or in combination with increasing amounts of PAP. Details as for (A; C) shows PAP levels in wild type and alx8 leaves and seeds. Data are means±SD, n=3.

FIG. 25—Shows stomatal aperture, calculated using measurements of pore width and length, in leaf peels of wild type plants treated with either mock measuring buffer (MB), 10 μM cordycepin, 100 μM PAP or 10 μM cordycepin and 100 μM PAP over a period of 1 hr. Statistically significant differences between treatments (p<0.05) are indicated by #,& for the 20-30 minute timeframe and a, b for the 40-60 minute timeframe.

FIG. 26—Shows stomatal closure in 5 week-old wild type plants sprayed with 100 mM PAP, PAP analogues or an equivalent volume of mock buffer.

FIG. 27—Shows inhibition of in vitro SAL1 activity against 50 μM PAP in the presence of increasing concentrations of Analogue 12.

FIG. 28—Shows accumulation of three PAP analogues in leaf tissue of Arabidopsis plants at various time points after spraying.

### DEFINITIONS

As used herein, the term “comprising” means “including principally, but not necessarily solely”. Variations of the word “comprising”, such as “comprise” and “comprises”, have correspondingly similar meanings.

As used herein the term “gene”, refers to a defined region that is located within a genome and that may comprise regulatory, nucleic acid sequences responsible for the control of expression, i.e., transcription and translation of the coding portion. A gene may also comprise other 5′ and 3′ untranslated sequences and termination sequences. Further elements that may be present are, for example, introns and coding sequences.

As used herein, the term “analogue” in the context of a peptide or protein means an artificial or natural substance that resembles the peptide or protein in function. For example, an enzyme analogue will bind the enzyme's substrate and thereby bring about the same or similar result as if the natural enzyme had bound the substrate. In an embodiment such analogues may also resemble the enzyme in structure. Analogues contemplated in an embodiment of the present invention include fully or partially peptidomimetic compounds as well as peptides or proteins resembling a subject peptide in activity but comprising addition, deletion, or substitution of one or more amino acids compared to the subject peptide or protein. The term “analogue” as used herein with reference to nucleotide sequences encompasses sequences comprising addition, deletion, or substitution (including conservative amino acid substitutions) of one or more bases relative to a subject nucleotide sequence, wherein the encoded polypeptide resembles the polypeptide encoded by the subject nucleic acid molecule in function.

As used herein, the term “homologue” in the context of proteins means proteins having substantially the same functions and similar properties in different species, and which, within at least regions, share at least 50% amino acid identity. Such homologous proteins may share, over their entire amino acid sequences, at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity. Similarly, homologues of nucleic acid molecules are nucleic acid molecules that encode proteins having substantially the same functions and similar properties in different species, wherein the encoded proteins share, within at least regions, at least 50% amino acid identity (such nucleic acid homologues may share significantly less than 50% identity due to degeneracy in the genetic code, and differences in preferred codon usage amongst different genuses and species), and may share at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity over the whole encoded amino acid sequences.

As used herein, the term “derivative or analogue thereof” in the context of 3′-phosphoadenosine-5′-phosphate (PAP) signaling means a substance that shares one or more aspects of the stress-signaling attributes of PAP such that the derivative or analogue thereof can at least partially mediate a stress response when present at sufficient levels. An analogue of PAP need not share any chemical or physical homology with PAP, but interacts with one or more of the same stress-signaling components that PAP interacts with in order to at least partially mediate a stress response effected by PAP.

As used herein, the term “PAP derivative or analogue thereof” in the context of 3′(2′),5′-bisphosphate nucleotidase inhibition means a substance that is capable of binding to the active site of a protein with 3′(2′),5′-bisphosphate nucleotidase activity and negatively affecting at least the 3′(2′),5′-bisphosphate nucleotidase activity of said protein. Such a derivative or analogue thereof need not share any stress-signaling attributes with PAP.

As used herein, the term “3′(2′),5′-bisphosphate nucleotidase activity” means an enzymatic activity that catalyses the reaction

adenosine 3′,5′-bisphosphate+H₂O→AMP+phosphate

and may be a SAL protein.

As used herein, the term “SAL protein” means a protein having at least 3′(2′),5′-bisphosphate nucleotidase activity and having an amino acid sequence comprising the amino acid sequence shown in SEQ ID NO:1, or the mature protein processed therefrom and lacking the secretion sequence, or any analogues or homologues thereof.

“Conservative amino acid substitutions” refer to the interchangeability of residues having similar side chains. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. Typically, conservative amino acids substitution groups are: valine-leucine-isoleucine, phenylalanine-tyrosine, lysine-arginine, alanine-valine, and asparagine-glutamine. Typically, conservative amino acid substitution(s) will result in a protein or polypeptide retaining at least some of the biological activity of the protein or polypeptide without such a conservative amino acid substitution. More typically, conservative amino acid substitution(s) will result in a protein or polypeptide having substantially the same, or at least comparable biological activity as the protein or polypeptide without such a conservative amino acid substitution. Conservative amino acid substitution(s) may result in proteins or polypeptides having greater biological activity than the protein or polypeptide without such a conservative amino acid substitution.

As used herein, the term “inhibitor” in the context of a biological activity relates to a substance that at least partially reduces that biological activity. Reduction of the biological activity may be through any mechanism, including interaction with the active site within a biological molecule (either reversibly or irreversibly), steric hindrance, conformational changes (including interference with or inhibition of conformational changes which a biological molecule usually undergoes when performing its biological activity), polymerisation (ie. promoting formation of dimers, trimers, tetramers, etc. of the biological molecule), or denaturation or degradation of the biological molecule, or any combination of such mechanisms. “Inhibits” and “reduces” and corresponding terms as used herein in the context of biological activity have corresponding meanings. Conversely, “enhances” and “promotes” and corresponding terms as used herein in the context of biological activity have converse meanings.

As used herein, the term “mutation” means any change in a polypeptide or nucleic acid molecule relative to a wild-type polypeptide or nucleic acid molecule from which the ‘mutant’ is derived and may, for example, comprise single or multiple amino acid or nucleotide changes, or both nucleotide and amino acid changes, including point mutations, null mutations, frame-shift mutations, and may comprise deletions, or insertions, or substitutions of one or more nucleic acids or amino acids, which may comprise naturally or non-naturally occurring nucleotides or amino acids or analogues thereof.

A “nucleic acid”, as referred to herein, refers to deoxyribonucleotides or ribonucleotides and polymers thereof in either single-, double-stranded or triplexed form. The term may encompass nucleic acids containing known analogues of natural nucleotides having similar binding properties as the reference nucleic acid. A particular nucleic acid sequence may also implicitly encompass conservatively modified variants thereof (e.g. degenerate codon substitutions) and complementary sequences. The terms “nucleic acid”, “nucleic acid sequence” or “polynucleotide” may also be used interchangeably with gene, cDNA, and mRNA encoded by a gene.

The terms “polypeptide”, “peptide” and “protein” may be used interchangeably herein to refer to a polymer of amino acid residues. Included within the scope of these terms are polymers in which one or more amino acid residues may comprise artificial chemical analogue(s) of corresponding naturally occurring amino acid(s), as well as, or instead of naturally occurring amino acid polymers. The terms “polypeptide”, “peptide” and “protein” may also include polymers including modifications, including post-translational modifications, such as, but not limited to, glycosylation (including arabinosylation), lipid attachment, sulfation, phosphorylation, gamma-carboxylation of glutamic acid residues, hydroxylation and ADP-ribosylation. Modified amino-acids may include further modifications. For example, hydroxylated residues may be glycosylated, such as arabinosylated hydroxyproline residues.

Within the scope of the terms “protein”, “polypeptide”, “polynucleotide” and “nucleic acid” as used herein are fragments and variants thereof, including but not limited to reverse compliment and antisense forms of polynucleotides and nucleic acids.

### DETAILED DESCRIPTION OF THE INVENTION

The present invention relates to methods for increasing plant stress responses, to create plants which, compared to untreated or unmodified plants, have greater above-ground biomass yield and/or faster development timelines when grown under stressful conditions, especially abiotic stress conditions, such as increased salinity, increased sodium levels, drought conditions, pH stress, and high light conditions. Thus, in embodiments, the plants yield a greater amount of above-ground plant matter than an untreated or wild-type plant grown under the same conditions. According to another embodiment, the plants grow faster than an untreated or wild-type plant grown under the same conditions. According to another embodiment, the plants develop faster than an untreated or wild-type plant grown under the same conditions. According to another embodiment, the plants survive longer than an untreated or wild-type plant grown under the same conditions. According to another embodiment, the plants are photosynthetically viable for longer than an untreated or wild-type plant grown under the same conditions. According to another embodiment, the plants retain more water than an untreated or wild-type plant grown under the same conditions. According to another embodiment, the plants prevent loss of above-ground matter compared to an untreated or wild-type plant grown under the same conditions.

The present studies have shown that PAP plays an important role in stress signalling in plants, and its levels correlate directly with stress tolerance by plants. PAP appears to function as a secondary messenger that acts in multiple cells, tissues and species, providing a previously unanticipated additional level of input and control into ABA-mediated signaling. The interaction between PAP and ABA signaling indicates that input from chloroplasts, in response to stimuli such as oxidative stress, can be incorporated into other cellular responses to drought. PAP may also be acting through other cellular compartments, or its levels may be affected by activities in other cellular compartments.

### Methods for Increasing Plant Stress Resistance

In agriculture it would be desirable to be able to produce plants which are better capable of tolerating stressful conditions while minimising effects on growth (and rates of growth) and biomass yield, and which continue to grow notwithstanding at least short term stress conditions. Alternatively, plants with increased stress sensitivity may also be of interest or of commercial value.

One manner of achieving such modulated stress tolerance may be through modulation of the levels of 3′-phosphoadenosine-5′-phosphate (PAP) or derivatives or analogues thereof in the cells of plants.

According to an aspect, methods of the present invention for increasing stress tolerance of a plant, said method comprising increasing the levels of 3′-phosphoadenosine-5′-phosphate (PAP) or a derivative or analogue thereof in the cells of said plant. While not wishing to be limited by theory, it is believed that PAP exerts its effects in the nucleus, and therefore, in an embodiment, PAP or a derivative or analogue thereof is accumulated in the nucleus of plant cells.

The methods of the invention may be carried out in any manner which results in increased levels of PAP in the plant cells, or in one or more compartments thereof.

For example, PAP or a derivative or analogue thereof may be applied to the plant by, for example, foliar spray or by application to the soil the plant is located in. In the case of foliar or stem application, the PAP or derivative or analogue thereof may be applied in any suitable composition as known in the art, and may include any suitable permeation enhancer or other substance that facilitates transfer of the PAP or analogue thereof into the plant cells. For example, a composition comprising PAP or a derivative or analogue thereof may comprise an abrasive agent (such as silica, carborundum or other hard substance), or may comprise a wetting agent (such as a detergent or other surfactant as known in the art).

Alternatively, a method of the present invention may comprise administering to the plant a substance that:
(i) enhances or promotes synthesis or accumulation of PAP or said derivative or analogue thereof; or
(ii) inhibits or reduces an activity metabolising or removing PAP or said derivative or analogue thereof.

Any substance that may lead to enhancement or promotion of synthesis or accumulation of PAP or a derivative or analogue thereof, or which inhibits or reduces an activity metabolising or removing PAP or a derivative or analogue thereof in plant cells (or one or more compartments thereof) is contemplated by the present invention.

Of particular interest are substances that may be involved in the synthesis of PAP or its precursors (that is, any substrate upstream of PAP in its synthetic pathway(s)), or substances that otherwise enhance the activity of an enzyme in the PAP biosynthetic pathway, or a pathway associated with the synthesis of a derivative or analogue of PAP.

Also of particular interest is any substance that inhibits an activity removing PAP or a derivative or analogue thereof from a cell or a compartment thereof, thereby depleting PAP or a derivative or analogue thereof in the cell or compartment rather than allowing PAP or a derivative or analogue thereof to accumulate. Such activities may be capable of catabolising PAP or a derivative or analogue thereof, or converting it to a different compound, or of transporting the PAP or a derivative or analogue thereof out of the cell, or a compartment thereof. An example of a substance that inhibits an activity removing PAP or a derivative or analogue thereof is a substance that inhibits or reduces the activity of a 3′(2′),5′-bisphosphate nucleotidase.

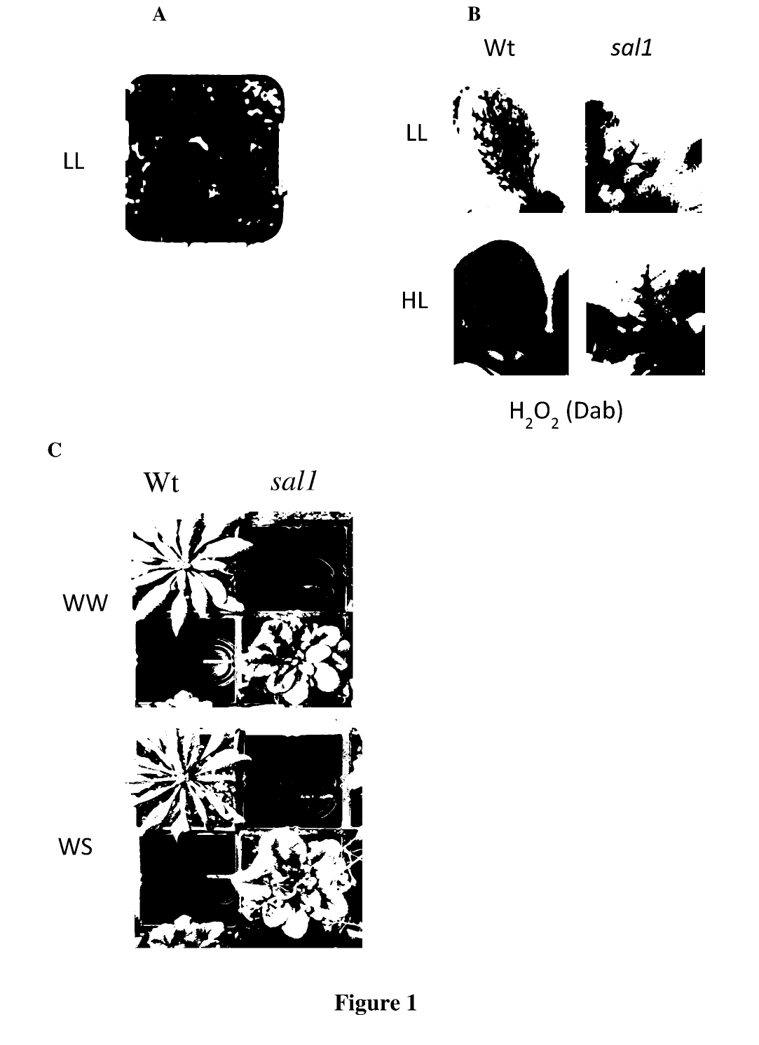

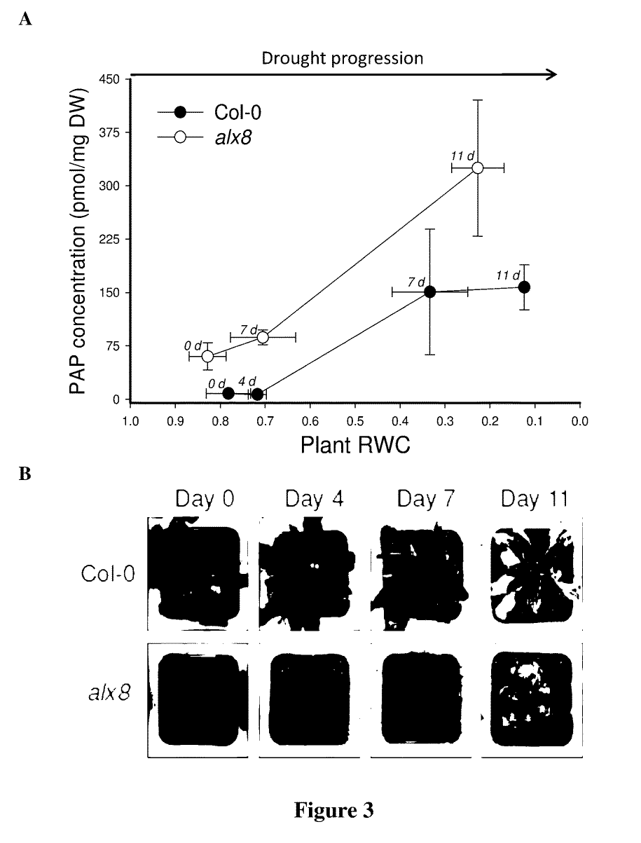

SAL1 is a plant protein that is bifunctional, with inositol polyphosphate 1-phosphatase activity and 3′(2′),5′-bisphosphate nucleotidase activity, and is involved in the catabolism of IP₃, a small molecule implicated in stress signalling, as well as PAP. It is the latter activity that, through the present studies, appears to be involved in stress-signalling. As shown in FIGS. 1 and 3, the sal1 mutant alx8, which does not express an active SAL1 protein, is more tolerant to at least high light and drought stress, as well as other oxidative stress conditions.

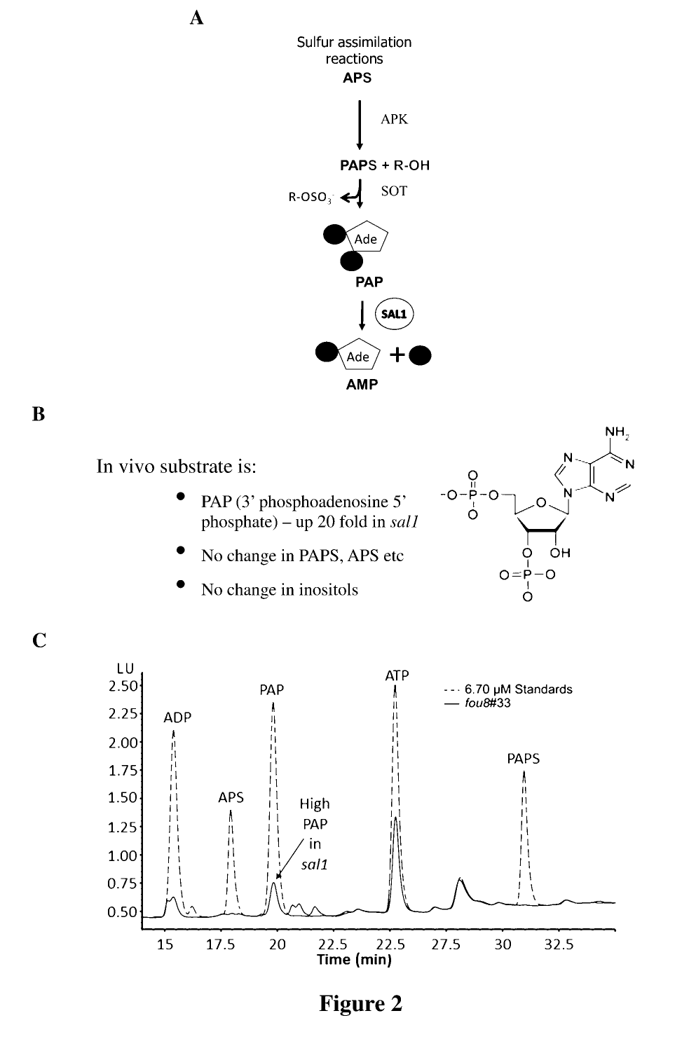

As shown in FIG. 2, SAL1 plays a significant role in sulfur assimilation reactions and in the catabolism of PAP. Cells of the alx8 mutant, showed significantly greater accumulation of PAP but, however, had normal PAPS, APS and inositol levels.

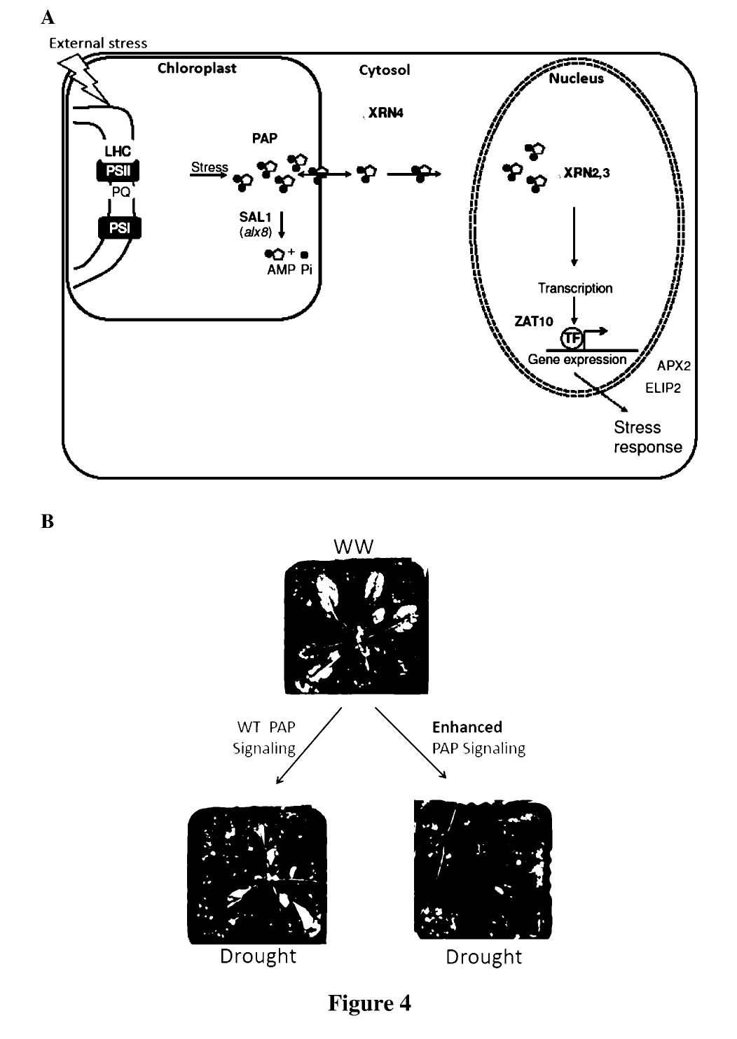

FIG. 4A shows a possible stress-signaling PAP-dependent pathway (acting through transcriptional regulation mediated by PAP, XRN2.3 and ZAT10), showing the role of SAL1 in controlling that response pathway through catabolism of PAP.

Thus, of particular interest are inhibitors of SAL1, or homologues thereof, or substances that reduce the activity thereof, through inhibition or reduced levels of active SAL1. Thus, in an embodiment, the substance is selected from PAP analogues or derivatives, or lithium or sodium ions.

Through the present studies, SAL1 has been found to have cysteine residues at the protein surface and therefore exposed to the cellular environment. Those cysteine residues are prone to oxidation and may also allow for dimerisation, or even formation of trimers and tetramers (or higher polymers) with reduced specific activity compared to the monomeric protein. During the course of these studies, it has been found that under oxidative conditions, the activity of these different forms of SAL1 is decreased. Thus, in another embodiment, the substance induces increased oxidative conditions inside said cells. In another embodiment, the substance induces reactive oxygen species inside said cells.

Thus, in another embodiment, the substance induces:

at least partial inactivation of a SAL protein;

(ii) oxidative conditions inside said cells;

(iii) conformational changes, polymerization, denaturation, degradation, or any combination thereof of a SAL protein;

(iv) reactive oxygen species inside said cells; or

(v) any combination of (i) to (iv).

Alternatively, a method of the present invention may comprise genetic modification of plants to allow accumulation of PAP or a derivative or analogue thereof in cells of plants, or compartments of said cells, by genetically impairing PAP (or a derivative or analogue thereof) catabolism or transport out of the cell or compartment, or by genetically enhancing synthesis in, or retention by said cells/compartments of PAP or a derivative or analogue thereof. Alternatively, the method may comprise mutating an endogenous sequence to make the encoded protein more susceptible to oxidative damage, dimerisation, denaturation, conformational change, degradation or any combination thereof, especially under stress conditions, such as abiotic stress conditions. For example, a method of the invention may comprise
(i) introducing a mutation in an endogenous nucleotide sequence;
(ii) replacing an endogenous nucleotide sequence with an exogenous nucleotide sequence; or
(iii) inserting an exogenous nucleotide sequence;
   in cells of said plant, wherein the resulting reduced activity, replaced activity or increased activity in said cells associated with said mutation or exogenous nucleotide sequence results in increased accumulation of PAP or said derivative or analogue thereof under at least stress conditions compared to a wild-type plant.

Of particular interest is a method which comprises:
(i) introducing a mutation in an endogenous 3′(2′),5′-bisphosphate nucleotidase activity-encoding sequence, optionally a SAL-encoding sequence; or
(ii) replacing an endogenous 3′(2′),5′-bisphosphate nucleotidase-encoding sequence, optionally an endogenous SAL-encoding sequence, with an exogenous 3′(2′),5′-bisphosphate nucleotidase-encoding sequence, optionally an exogenous SAL-encoding sequence;
   into said cells, whereby the 3′(2′),5′-bisphosphate nucleotidase activity, optionally SAL activity, in said cells is at least reduced under at least stress conditions compared to a wild-type plant.

Preferably the 3′(2′),5′-bisphosphate nucleotidase is a SAL protein, and said mutated endogenous SAL-encoding sequence or exogenous SAL-encoding sequence encode a SAL protein comprising cysteine residues at positions equivalent to one or more of positions 173, 221 and 244 of SEQ ID NO:1. According to another embodiment of this aspect, the method further comprises administering to said plant a substance that induces increased oxidative conditions inside said cells.

According to an embodiment, the mutation in a SAL protein or homologue thereof is not a known mutation, including the Arabidopsis thaliana fry, fry1, fiery, hos, hos2, salk02882, or alx8 mutations.

### PAP and Derivatives and Analogues

Any substance that shares one or more aspects of the stress-signaling attributes of PAP is contemplated by the present invention such that the derivative or analogue thereof can at least partially mediate a stress response when present at sufficient levels. Thus, such a derivative or analogue may at least partially increase stress tolerance of a plant compared to an untreated plant. An analogue of PAP need not share any chemical or physical homology with PAP, but interacts with one or more of the same stress-signaling components that PAP interacts with in order to at least partially mediate a stress response effected by PAP. Examples of PAP analogues for use in methods according to the present invention include 3′-deoxyadenosine (cordycepin; i.e. Analogue 16) as well as analogues 4, 5, 7, 8, 11, 12, 13, 14 as identified in Table 1 below.

Table 1 provides a non-exhaustive list of potential PAP derivatives or analogues.

TABLE 1
Non-exhaustive examples of structural analogues of PAP, which may have the same function as PAP in vivo.

| Name | Mol Weight | Structure |
|---|---|---|
| 3′-phosphoadenosine 5′- phosphate PAP), alternative name: Adenosine 3′,5′- diphosphate | 423.1 | 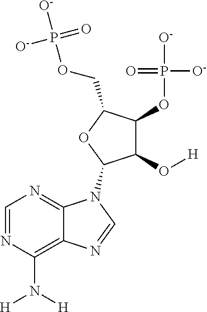 [SMILES: Nc1ncnc2c1ncn2[C@@H]1O[C@H](COP(=O)([O-])[O-])[C@@H](OP(=O)([O-])[O-])[C@H]1O] |
| Adenosine 2′,5′- diphosphate (2′-PAP) | 423.1 | 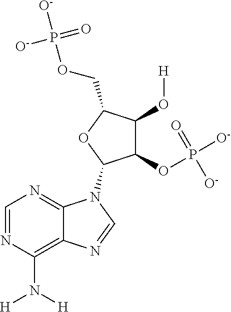 [SMILES: Nc1ncnc2c1ncn2[C@@H]1O[C@H](COP(=O)([O-])[O-])[C@@H](O)[C@H]1OP(=O)([O-])[O-]] |
| 3′-phosphate-adenosine- 5′-diphosphate | 507.1 | 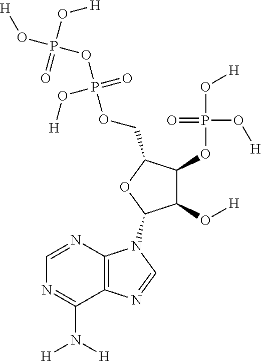 [SMILES: Nc1ncnc2c1ncn2[C@@H]1O[C@H](COP(=O)(O)OP(=O)(O)O)[C@@H](OP(=O)(O)O)[C@H]1O] |
| 3′-phosphoadenosine-5′- phosphosulfate (PAPS) | 507.2 | 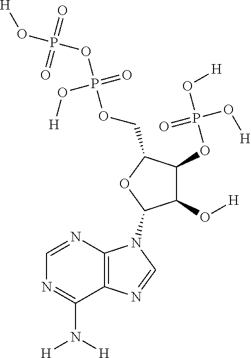 |
| PAP-Malonyl-CoA | 1255.7 | 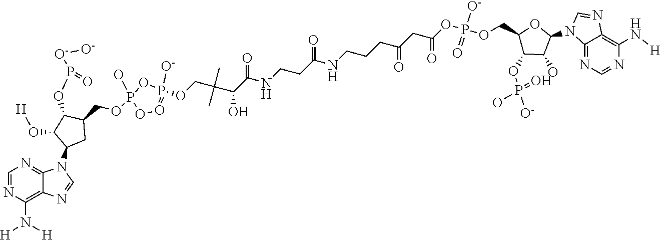 |
| Adenosine 5′ Monophosphate (5′- AMP) | 347.22 | 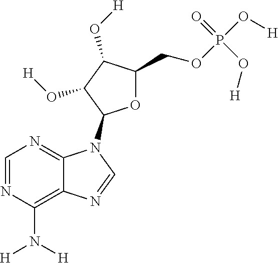 [SMILES: Nc1ncnc2c1ncn2[C@@H]1O[C@H](COP(=O)(O)O)[C@@H](O)[C@H]1O] |
| Adenosine 3′ Monophosphate (3′- AMP) | 345.2 | 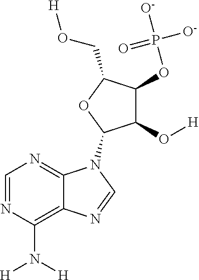 [SMILES: Nc1ncnc2c1ncn2[C@@H]1O[C@H](CO)[C@@H](OP(=O)([O-])[O-])[C@H]1O] |
| N-[9-(5-{[bis(4- methoxyphenyl)(phenyl) methoxy]methyl}1-3,4- dihydroxytetrahydro-2- furanyl)-9H-purin-6- yl]benzamide [Analogue 1] | 673.7 | 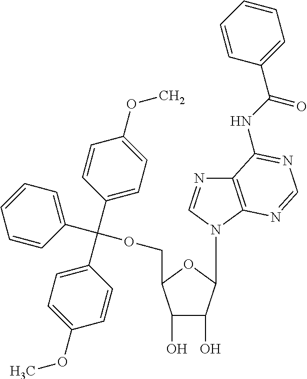 [SMILES: COc1ccc(C(OCC2OC(n3cnc4c(NC(=O)c5ccccc5)ncnc43)C(O)C2O)(c2ccccc2)c2ccc(OC)cc2)cc1] |
| 2-{[bis(4- methoxyphenyl)(phenyl) methoxy]methyl}-5-(6- hydroxy-9H-purin-9- yl)tetrahydro-3,4- furandiol [Analogue 2] | 570.6 | 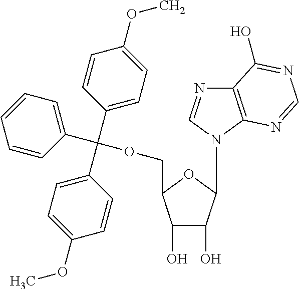 [SMILES: COc1ccc(C(OCC2OC(n3cnc4c(O)ncnc43)C(O)C2O)(c2ccccc2)c2ccc(OC)cc2)cc1] |
| 2-{6-[(4- hydroxybutyl)amino]- 9H-purin-9-yl}-5- (hydroxymethyl) tetrahydro- 3,4-furandiol [Analogue 3] | 339.3 | 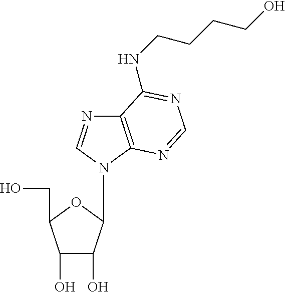 [SMILES: OCCCCNc1ncnc2c1ncn2C1OC(CO)C(O)C1O] |
| 2-{6-[(2- hydroxyethyl)amino]- 9H-purin-9-yl}-5- (hydroxymethyl) tetrahydro- 3,4-furandiol [Analogue 4] | 311.3 | 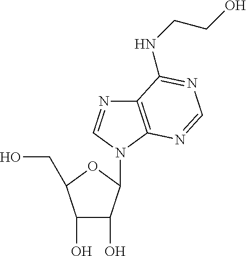 [SMILES: OCCNc1ncnc2c1ncn2C1OC(CO)C(O)C1O] |
| 2-[6-(diethylamino)-9H- purin-9-yl]-5- (hydroxymethyl) tetrahydro- 3,4-furandiol [Analogue 5] | 323.3 | 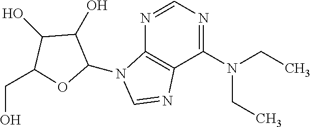 [SMILES: CCN(CC)c1ncnc2c1ncn2C1OC(CO)C(O)C1O] |
| 2-{2-amino-6-[(4- nitrobenzyl)thio]-9H- purin-9-yl}-5- (hydroxymethyl) tetrahydro- 3,4-furandiol [Analogue 6] | 434.4 | 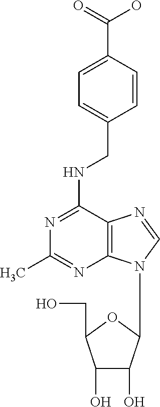 [SMILES: Cc1nc(NCc2ccc(C(=O)O)cc2)c2ncn(C3OC(CO)C(O)C3O)c2n1] |
| 2-(hydroxymethyl)-5-(6- mercapto-9H-purin-9- yl)tetrahydro-3,4- furandiol [Analogue 7] | 284.3 | 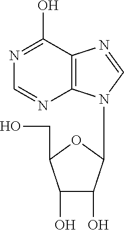 [SMILES: OCC1OC(n2cnc3c(O)ncnc32)C(O)C1O] |
| 5-(6-amino-9H-purin-9- yl)-N-ethyl-3,4- dihydroxytetrahydro-2- furancarboxamide [Analogue 8] | 308.3 | 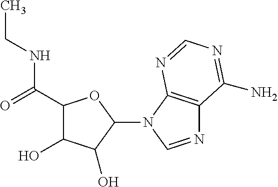 [SMILES: CCNC(=O)C1OC(n2cnc3c(N)ncnc32)C(O)C1O] |
| 6-(6-amino-9H-purin-9- yl)-2,2- dimethyltetrahydrofuro [3,4-d][1,3]dioxole-4- carboxylic acid [Analogue 9] | 321.3 | 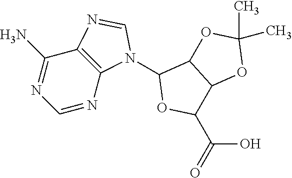 [SMILES: CC1(C)OC2C(C(=O)O)OC(n3cnc4c(N)ncnc43)C2O1] |
| 5-(6-amino-9H-purin-9- yl)-3,4- dihydroxytetrahydro-2- furancarboxylic acid [Analogue 10] | 281.2 | 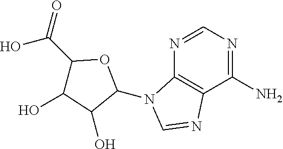 [SMILES: Nc1ncnc2c1ncn2C1OC(C(=O)O)C(O)C1O] |
| 2-[6-(dimethylamino)- 9H-purin-9-yl]-5- (hydroxymethyl) tetrahydro- 3,4-furandiol [Analogue 11] | 295.3 | 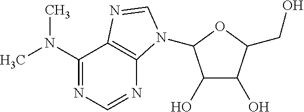 [SMILES: CN(C)c1ncnc2c1ncn2C1OC(CO)C(O)C1O] |
| 2-(6-amino-8-mercapto- 9H-purin-9-yl)-5- (hydroxymethyl) tetrahydro- 3,4-furandiol [Analogue 12] | 299.3 | 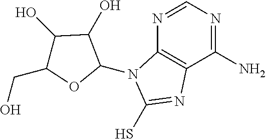 [SMILES: Nc1ncnc2c1nc(S)n2C1OC(CO)C(O)C1O] |
| methyl 5-(6-amino-9H- purin-9-yl)-3,4- dihydroxytetrahydro-2- furancarboxylate [Analogue 13] | 295.3 | 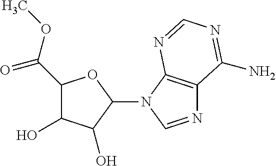 [SMILES: COC(=O)C1OC(n2cnc3c(N)ncnc32)C(O)C1O] |
| 2-[2-(acetylamino)-6- hydroxy-9H-purin-9-yl]- 5- [(acetyloxy)methyl]tetra- hydrofuran-3,4-diyl diacetate [Analogue 14] | 451.4 | 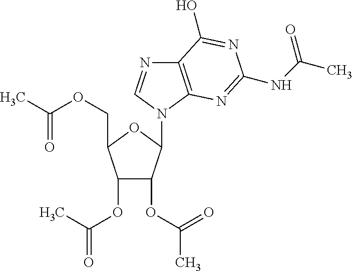 [SMILES: CC(=O)Nc1nc(O)c2ncn(C3OC(COC(C)=O)C(OC(C)=O)C3OC(C)=O)c2n1] |
| 3,4- dimethoxybenzaldehyde {6-amino-9-[3,4- dihydroxy-5- (hydroxymethyl)tetra- hydro-2-furanyl]-9H- purin- 8-yl}hydrazone [Analogue 15] | 445.4 | 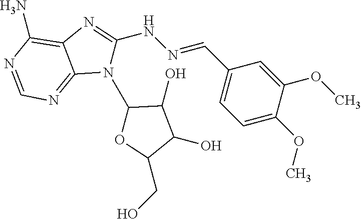 [SMILES: COc1ccc(/C=N/Nc2nc3c(N)ncnc3n2C2OC(CO)C(O)C2O)cc1OC] |
| 3′-deoxyadenosine (cordycepin) [Analogue 16] | 251.2 | 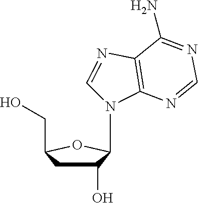 [SMILES: Nc1ncnc2c1ncn2[C@@H]1O[C@H](CO)C[C@@H]1O] |
| 8-azidoadenosine 3,5′- biphosphate [Analogue 17] | 468.2 | 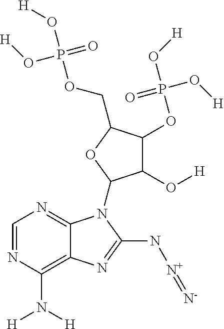 [SMILES: [N-]=[NH+]Nc1nc2c(N)ncnc2n1C1OC(COP(=O)(O)O)C(OP(=O)(O)O)C1O] |

### Inhibitors of Biological Activity Resulting in PAP Metabolism or Transport

Any substance that at least partially reduces a biological activity resulting in PAP metabolism or transport is contemplated by the present invention. Reduction of the biological activity may be through any mechanism, including interaction with the active site within a biological molecule (either reversibly or irreversibly), steric hindrance, conformational changes (including interference with or inhibition of conformational changes which a biological molecule usually undergoes when performing its biological activity), polymerisation (ie. promoting formation of dimers, trimers, tetramers, etc. of the biological molecule), or denaturation or degradation of the biological molecule, or any combination of such mechanisms.

Inhibitors of particular interest are any inhibitors that reduce the activity of a SAL protein (or homologue thereof) in a cell or compartment within that cell. Such a reduction in activity may occur through competitive, uncompetitive or noncompetitive inhibition, including steric hindrance, reversible or irreversible binding to the active site, or moieties involved in the activity, or may occur through physical damage to the activity, such as denaturation, conformational change, polymerisation or degradation.

Substances that induce formation of reactive oxygen species within the plant cells, or otherwise result in increased redox levels, or otherwise result in oxidative stress inside plant cells are contemplated by the present invention.

Table 2 provides a non-exhaustive list of compounds that may induce oxidative stress and production of reactive oxygen species in planta, which could be used to modulate SAL1 activity and chloroplast signaling.

TABLE 2
Summary of different herbicide classes and chemical families known to induce
oxidative stress and production of reactive oxygen species in vivo.

| Classification | | | |
|---|---|---|---|
| group* | Site of action | Chemical family | Common name |
| 1 | Inhibitors of acetyl CoA | Aryloxyphenoxy | Clodinafop‡ |
| | carboxylase | propion | Cyhalofop-butyl |
| | | | Diclofop |
| | | | Fenoxaprop |
| | | | Fluazifop-P |
| | | | Haloxyfop |
| | | | Propaquizafop |
| | | | Quizalofop-P |
| | | Cyclohexanedioneate | Alloxydim |
| | | | Butroxydim |
| | | | Clethodim |
| | | | Cycloxydim |
| | | | Sethoxydim |
| | | | Tralkoxydim |
| 5 | Inhibitors of | Phenyl-carbamate | Desmedipham |
| | photosynthesis at | | Phenmedipham |
| | photosystem II, site A† | | |
| | | Pyridazinone | Pyrazon |
| | | Triazine | Ametryn |
| | | | Atrazine‡ |
| | | | Cyanazine |
| | | | Desmetryn |
| | | | Prometon |
| | | | Prometryn |
| | | | Propazine |
| | | | Simazine |
| | | | Simetryn |
| | | | Terbumeton |
| | | | Terbuthylazine |
| | | | Trietazine |
| | | Triazinone | Hexazinone |
| | | | Metamitron |
| | | | Metribuzin |
| | | Triazolinone | Amicarbazone |
| | | Uracil | Bromacil |
| | | | Terbacil |
| 6 | Inhibitors of | Benzothiadiazole | Bentazon |
| | photosynthesis at | Nitrile | Bromoxynil |
| | photosystem II, site B† | | Ioxynil |
| | | Phenyl-pyridazine | Pyridate |
| 7 | Inhibitors of | Amide | Propanil |
| | photosynthesis at | Urea | Chlorotoluron |
| | photosystem II site A; | | Dimefuron |
| | different binding | | Diuron |
| | behavior | | Fluometuron‡ |
| | from group 5† | | Isoproturon |
| | | | Linuron |
| | | | Methibenzuron |
| | | | Metoxuron |
| | | | Monolinuron |
| | | | Siduron |
| | | | Tebuthiuron |
| 11 | Inhibitors of carotenoid | Triazole | Amitrole |
| | synthesis (unknown | | Aclonifen |
| | target)† | | |
| 12 | Inhibitors of Phytoene | Pyridazinone | Norflurazon |
| | Desaturase (PDS)† | Pyridinecarboxamide | Diflufenican |
| | | | Picolinafen |
| | | Other | Beflubtamid |
| | | | Fluridone |
| | | | Fluorochloridone |
| | | | Flurtamone |
| 13 | Inhibitor of 1-deoxy-D- | Isoxazolidinone | Clomazone |
| | xyulose 5-phos phate | | |
| | synthatase (DOXP | | |
| | synthase)† | | |
| 14 | Inhibitors of | Diphenylether | Acifluorfen |
| | protoporphyrin-ogen | | Bifenox |
| | oxidase (Protox) | | Fomesafen |
| | | | Fluoroglycofen |
| | | | Lactofen |
| | | | Oxyfluorfen‡ |
| | | N-phenylphthalimide | CGA-248757 |
| | | | Flumiclorac |
| | | | Flumioxazin |
| | | Oxadiazole | Oxadiazon |
| | | | Oxadiargyl |
| | | Phenylpyrazole | Pyraflufen-ethyl |
| | | Pyrimidindione | Butafenacil |
| | | Thiadiazole | Fluthiacet- |
| | | | methyl |
| | | Triazinone | Carfentrazone- |
| | | | ethyl |
| | | | Sulfentrazone |
| | | Triazolone | Azafenidin |
| | | Other | Flufenpyr-ethyl |
| 15 | Inhibitors of synthesis of | Acetamide | Napropamide |
| | very long-chain fatty acids | Chloroacetamide | Acetochlor |
| | | | Alachlor‡ |
| | | | Butachlor |
| | | | Dimethenamid |
| | | | Metolachlort |
| | | | Metazachlor |
| | | | Pretilachlor |
| | | | Propachlor |
| | | | Thenylchlor |
| | | Oxyacetamide | Mefenacet |
| | | | Flufenacet |
| | | Tetrazolinone | Fentrazamide |
| | | Other | Anilofos |
| 22 | Photosystem I electron | Bipyridylium | Diquat‡ |
| | diverters† | | Paraquat‡ |
| 27 | Inhibitors of 4- | Isoxazole | Isoxaflutole |
| | hydroxyhenyl-pyruvate | Pyrazole | Benzofenap |
| | dioxygenase (4-HPPD)† | | Pyrazolynate |
| | | | Pyrazoxyfen |
| | | Triketone | Mesotrione |
| | | | Sulcotrione |

*based on Weed Science Society of America (WSSA) nomenclature, Mallory-Smith & Retzinger 2003 Weed Technology 17: 605-619
†Classified as herbicides known to induce oxidative stress in plants by the Weed Science Society of America (WSSA)
‡Direct experimental evidence from the literature for induction of oxidative stress and production of reactive oxygen species in vivo upon application.

Modification of Proteins to Make them More Susceptible to Oxidative Stress

An aim of the present invention is to make biological activities responsible for catabolism of PAP or a derivative or analogue thereof, or for transport of PAP or a derivative or analogue thereof out of plant cells or a compartment thereof, more susceptible to conformational changes, polymerisation (ie. promoting formation of dimers, trimers, tetramers, etc. of the biological molecule), or denaturation or degradation of the biological molecule, or any combination of such mechanisms.

As illustrated in FIGS. 2, 4 and 7, SAL1 is involved in metabolism of PAP, being responsible for its dephosphorylation, whereas PAP is the active agent in stress signalling. Oxidative conditions, such as those that arise during periods of oxidative stress have been found to result in increased levels of PAP, presumably due to reduced activity of SAL1, and increased tolerance to abiotic stresses, such as drought tolerance.

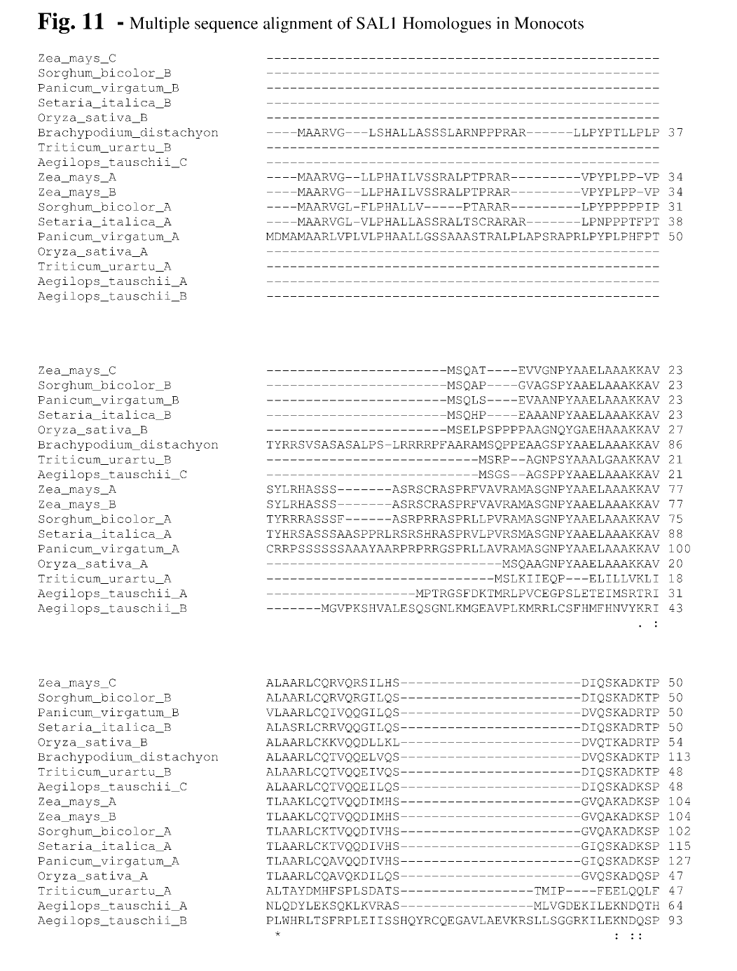

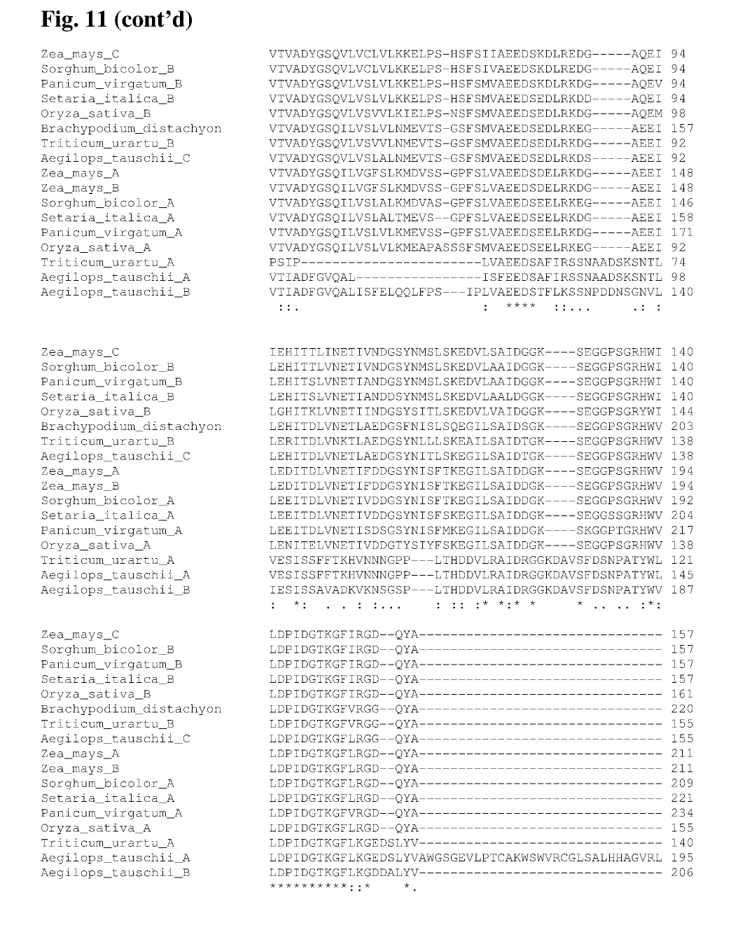

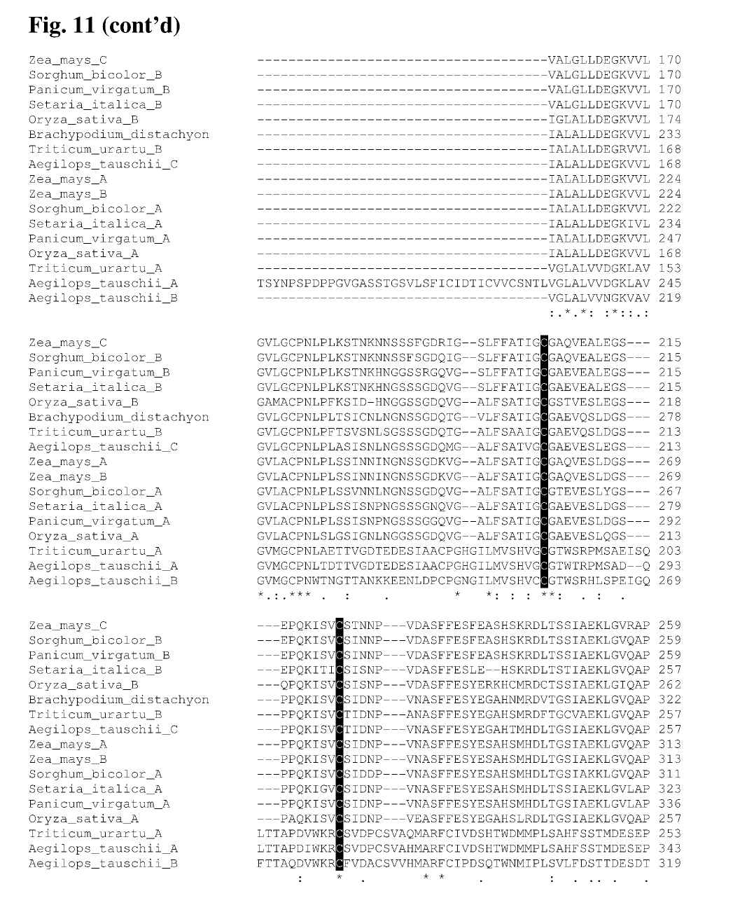

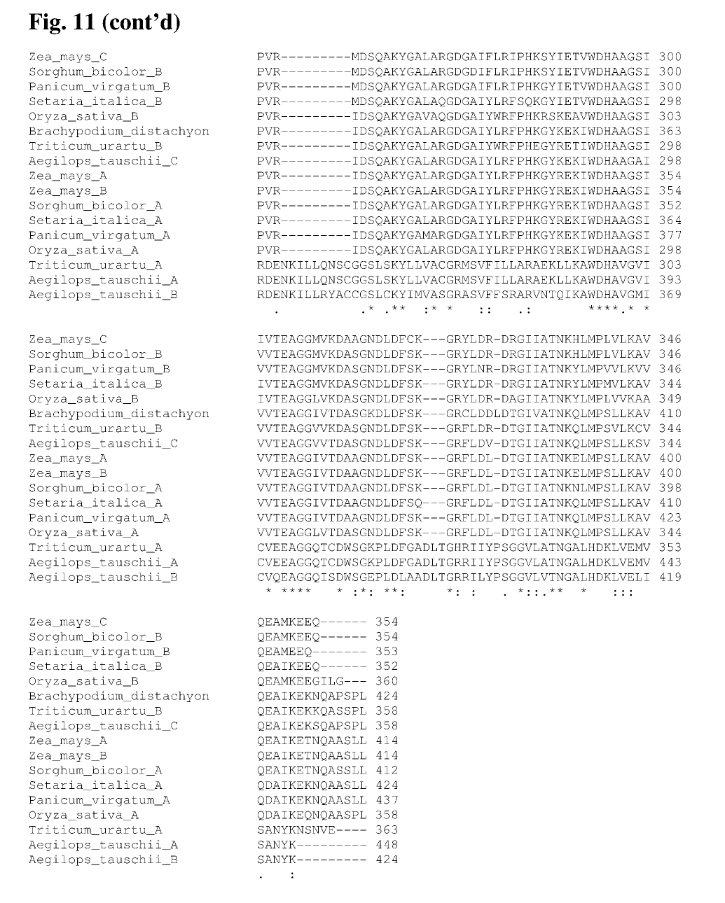

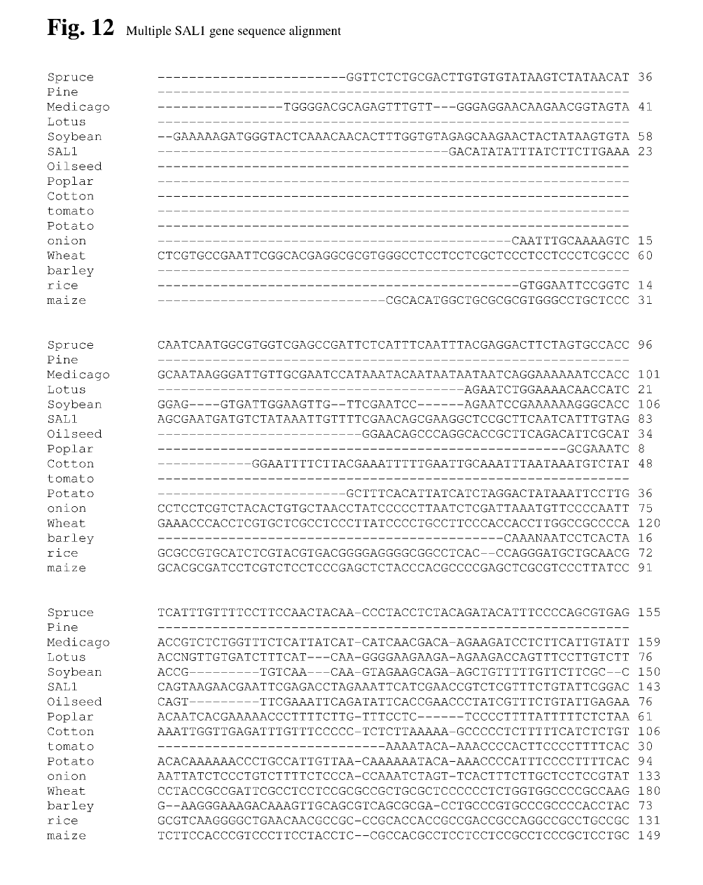

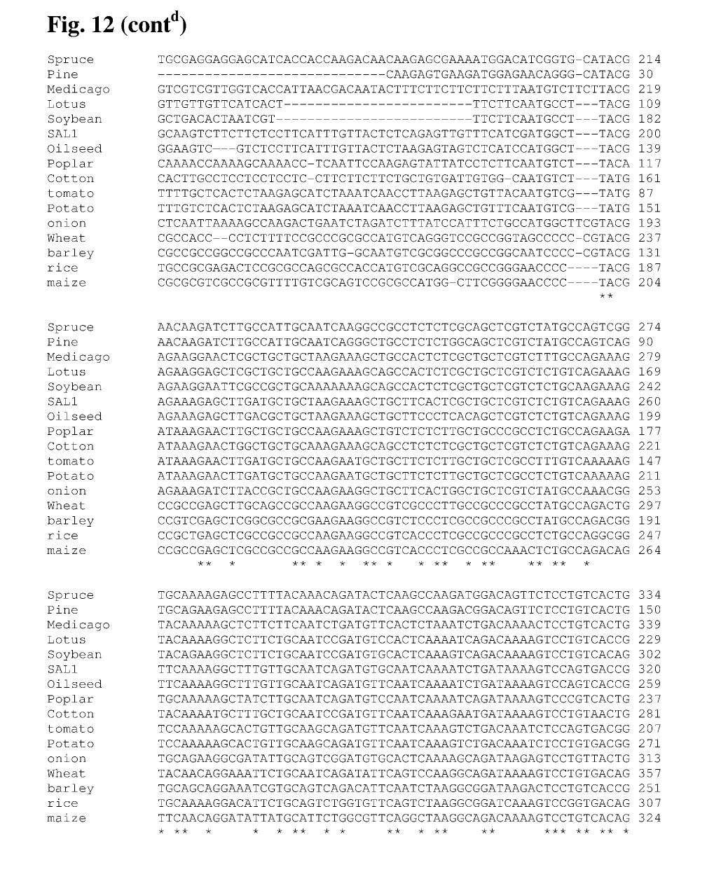

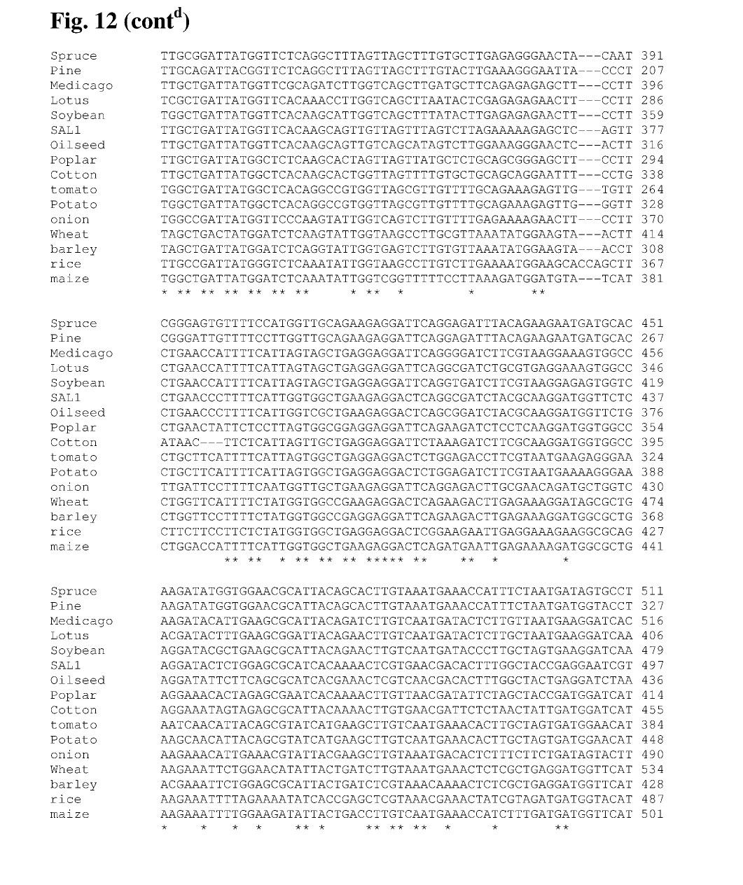

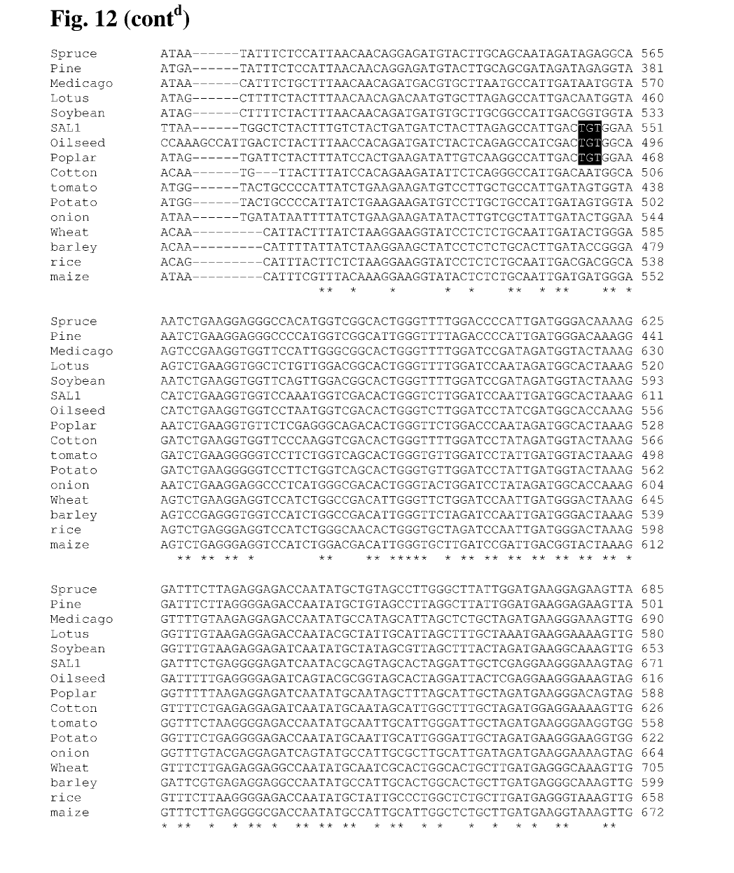

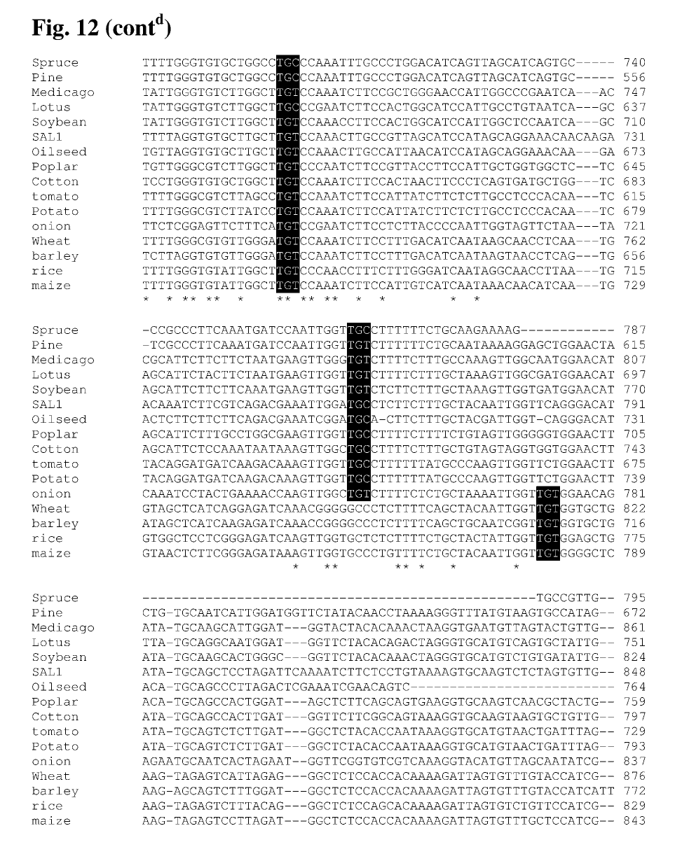

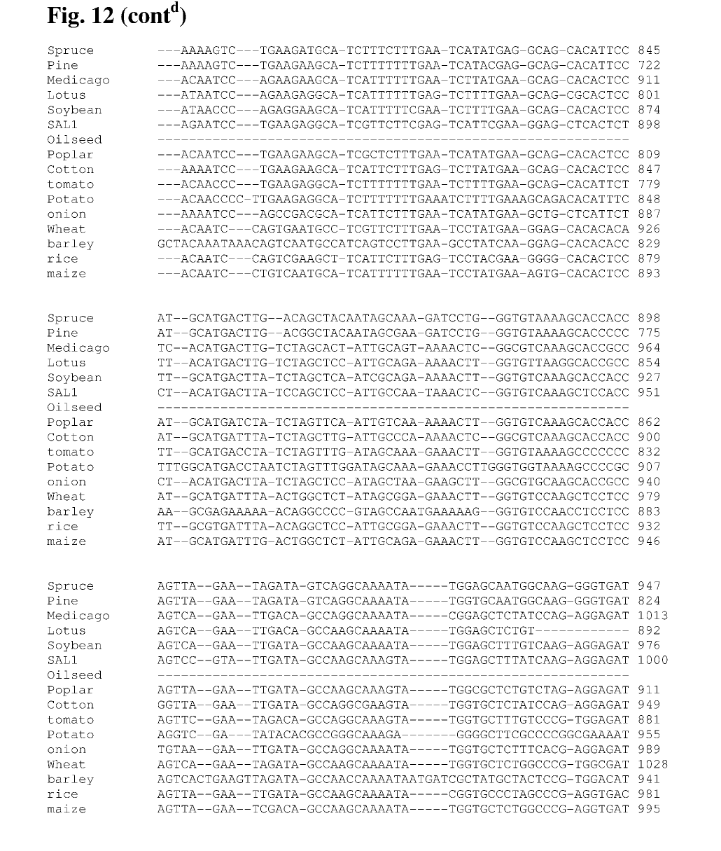

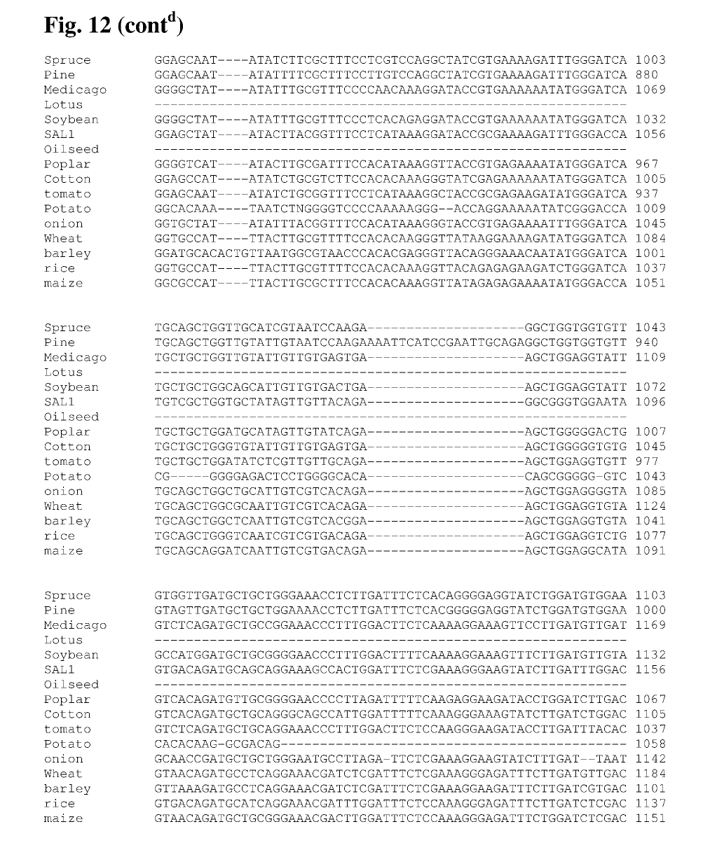

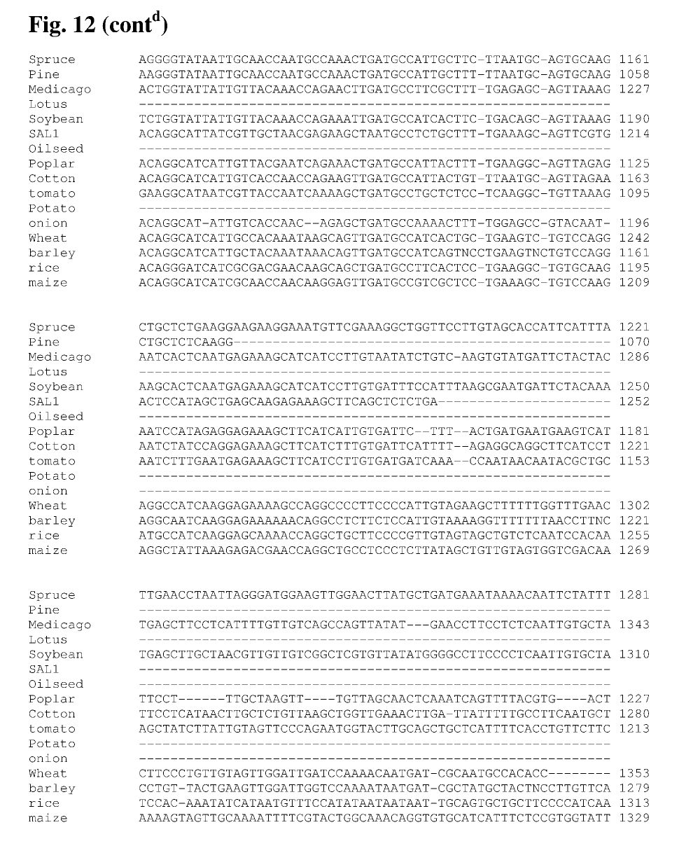

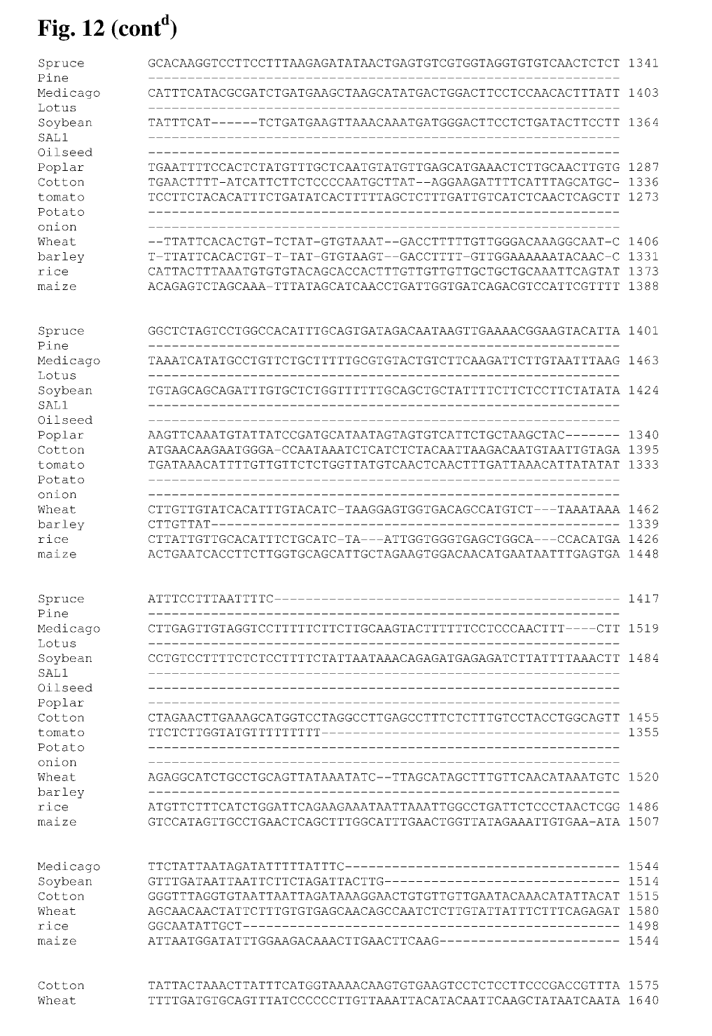

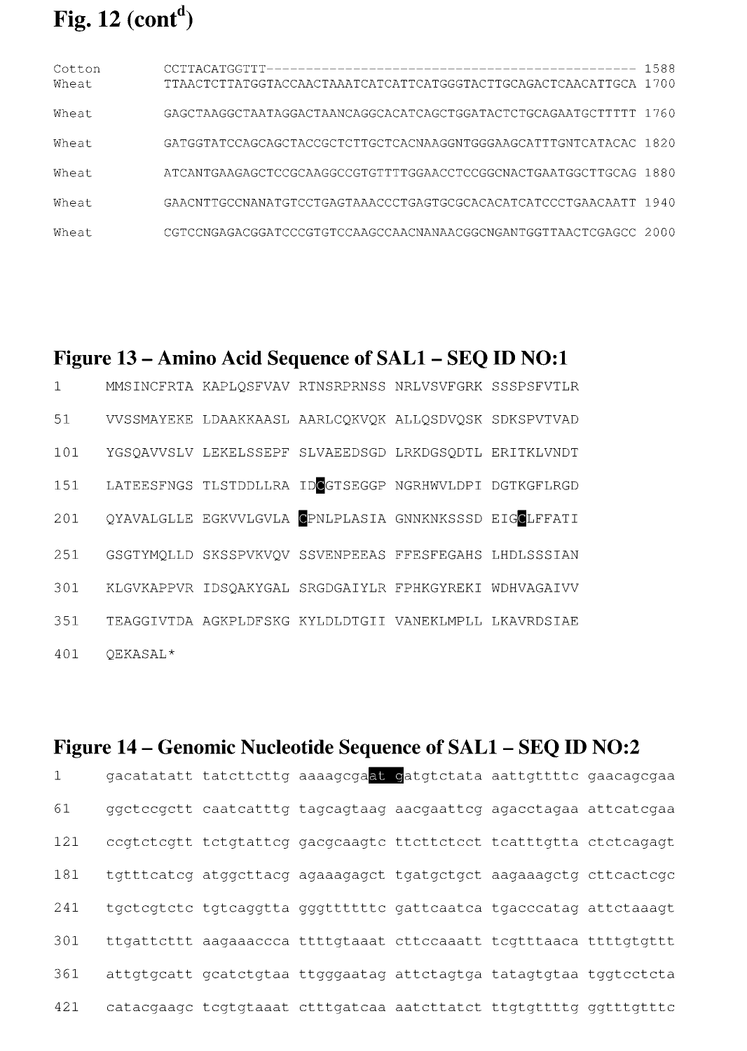

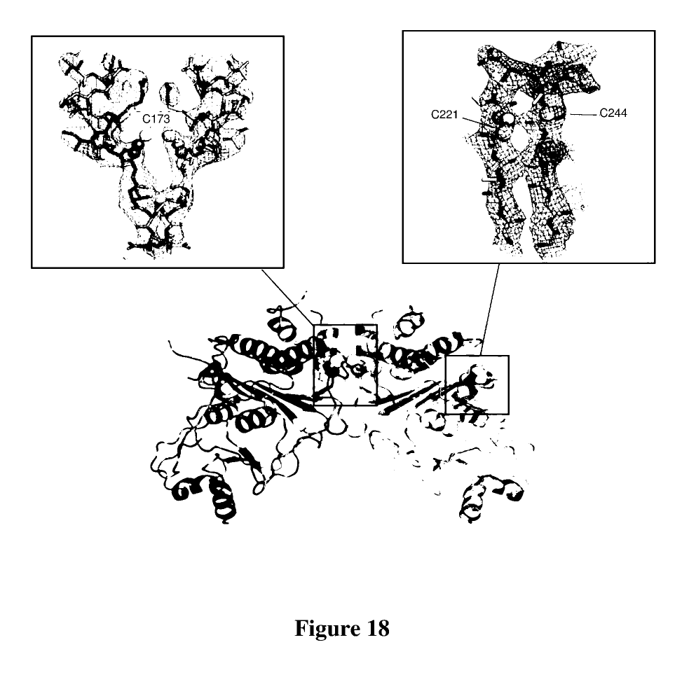

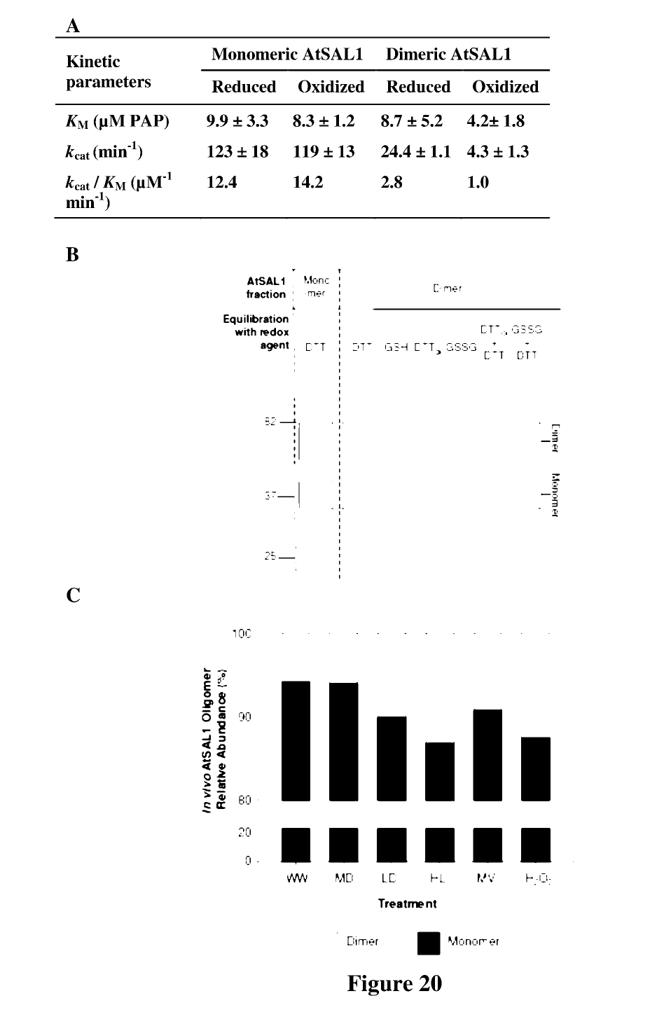

Preliminary modelling of a 4 Angstrom recombinant SAL1 (rSAL1) crystal structure (see FIG. 18) reveals both internal and surface-exposed, redox-sensitive cysteine residues that are highly conserved across the plant kingdom (both monocots and dicots—see FIGS. 11 and 12), including in crops such as rice and tomato. Oxidation in vitro significantly decreased activity of dimeric, but not monomeric, rSAL1 with up to 6-fold decrease in k_{cat} for the oxidized dimeric rSAL1 (FIG. 20A).

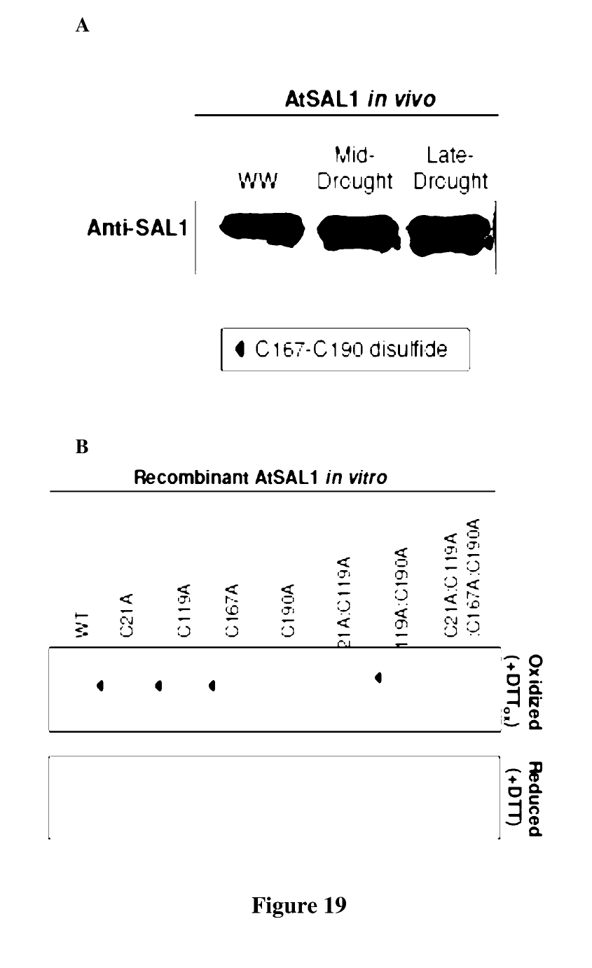

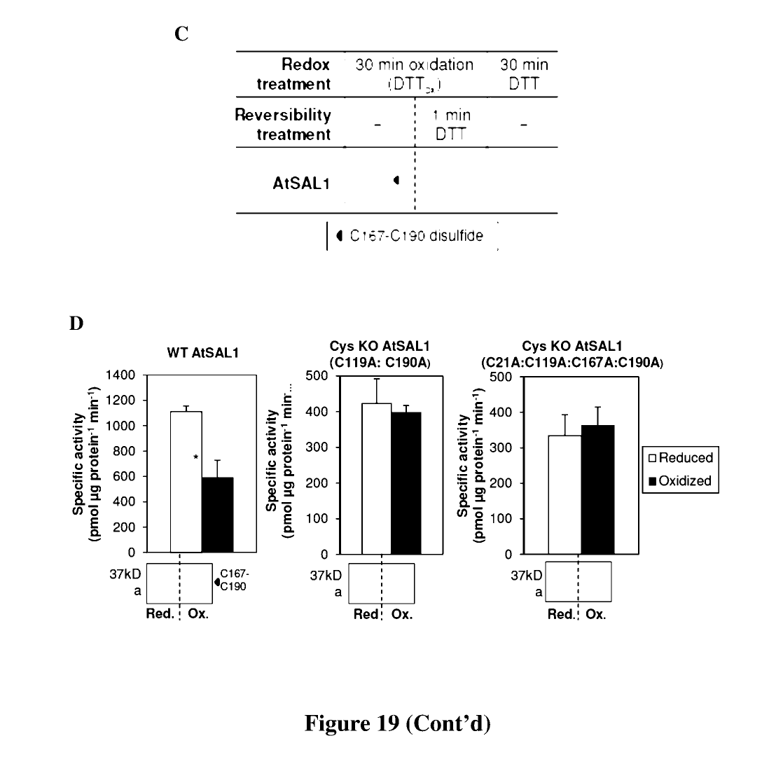

These observations indicate that oxidation of cysteine residues in SAL1 or homologues thereof, or other proteins, could lead to proteins which are more susceptible to oxidative conditions. Therefore, engineering specific residues important for oxidation (cysteines), optionally dimerization could confer a ‘switch’ for altered function and regulation. Indeed, mutagenesis of selected cysteine residues predicted to be redox sensitive have resulted in mutated rSAL1 proteins that were less sensitive to oxidation (FIG. 19).

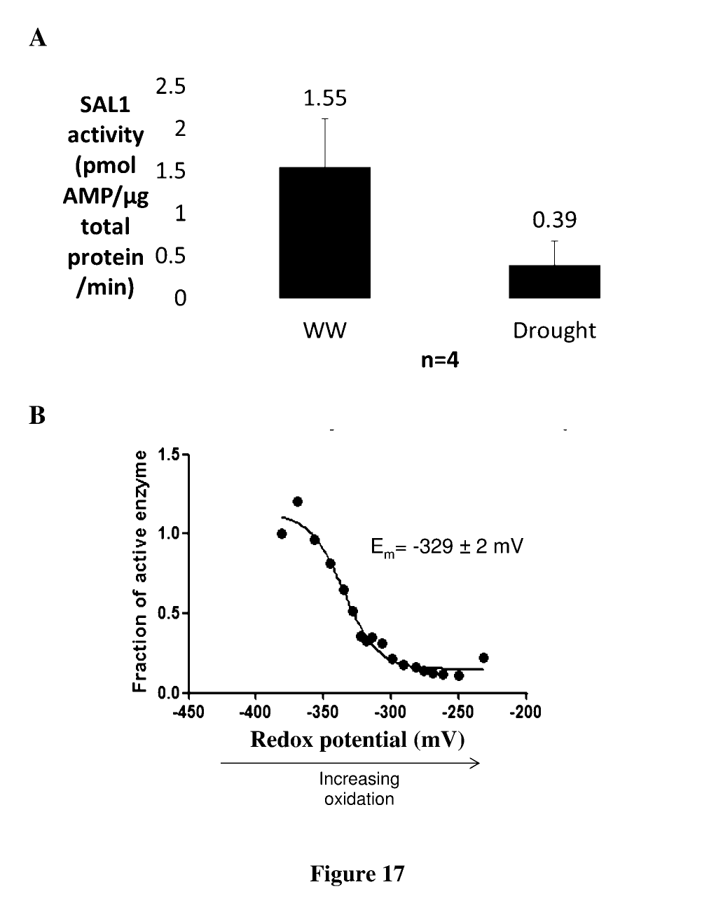

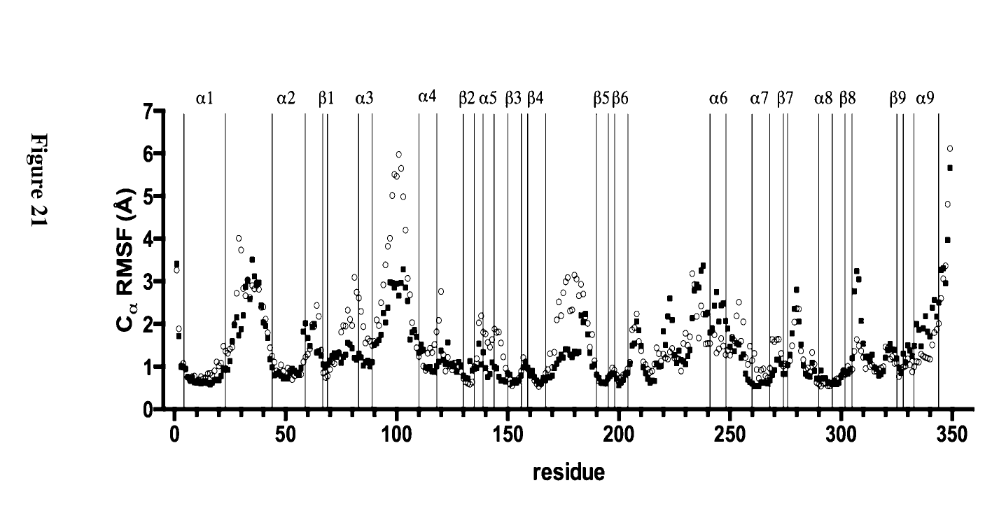

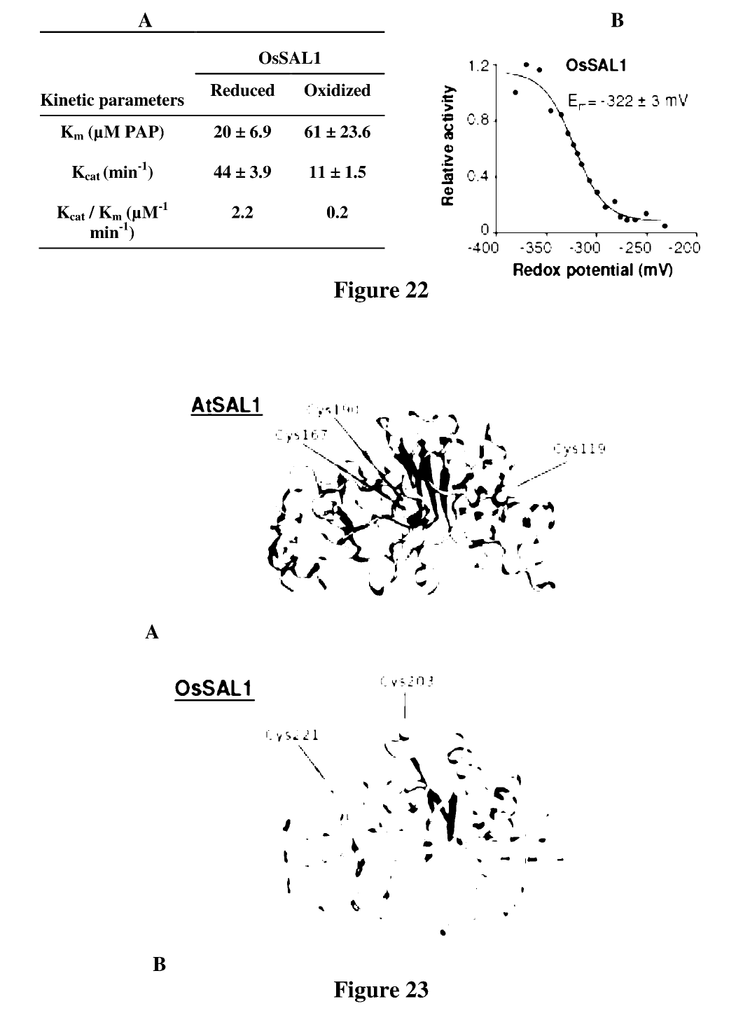

Finally, rSAL1 redox midpoint potential (Eₘ) was in the physiologically-relevant range and SAL1 activity was decreased during drought stress, suggesting that this mechanism can be extended to protein regulation in vivo (FIGS. 17 and 22).

This mechanism could be more broadly applied to provide a rapid, sensitive ‘switch’ to regulate enzymatic activity and function both in a controlled, user-defined manner in vitro and under physiologically-relevant stress conditions, especially abiotic stress conditions in vivo.

### Selecting Proteins for Transformation

SAL1 has been described herein as a model protein for exemplifying the present invention which, however, is not so limited. Proteins which share similar protein folds and therefore architecture as the model protein SAL1 can be identified by employing search algorithms to query the SAL1 structure against protein crystal structure databases, including the method by Krissinel and Henrick (2004), Acta Cryst. D 60: 2256-2268 incorporated herein in its entirety by cross-reference.

For proteins where the crystal structure is not available, possible protein structure can be first deduced by homology modelling to structurally-characterized family members of these target proteins using a variety of algorithms such as those by Schwede et al (2003), Nucleic Acids Research 31(13): 3381-3385; Kelley and Sternberg (2009), Nature Protocols 4: 363-371; Roy et al (2010) Nature Protocols, 5: 725-738; all of which are hereby incorporated herein in their entirety by cross-reference. The predicted protein structure can then be compared to the SAL1 structure to assess similarity in folds containing the redox-sensitive residues. For example, using the methods described above, proteins containing inositol phosphatase/kinase or fructose-1,6-bisphosphatase activity were identified as having similar folds to SAL1 and may therefore be amenable to modification.

However, proteins which do not have similar folds to SAL1 can still be engineered. For instance, the motifs/folds that are responsible for redox regulation in SAL1 can be inserted into these target proteins to produce hybrid proteins.

Position and type of residues to be modified or inserted in a particular target protein can be identified by analyzing the 3D structure of the protein, whether already available as a crystal structure or generated by homology modelling as described above. In both cases analysis of the structure will reveal, for instance, peptide loops which are already in close contact in 3D space. Amino acid residues in these loops will be candidates for replacement with cysteine residues for oxidation.

Introducing Modifications into Desired Polypeptides

Introducing modifications into target polypeptides may, for example, be achieved by modifications in the underlying nucleotide sequences encoding for said polypeptides, as described below. Introduction, or removal, of redox-sensitive cysteine residues in a target polypeptide can be achieved by mutagenesis of the underlying nucleotide sequence at the codon encoding for the target residue. This can be performed by site-directed mutagenesis (see Braman, Jeff ed. 2002, “In vitro Mutagenesis Protocols” in Methods in Molecular Biology, 182, 2^{nd} ed, Humana Press), domain-swapping (e.g. Atanassov et al (2009), Plant Methods 5:14), or chemical synthesis of the nucleotide sequence using established methods (see Herdewijn 2005, “Oligonucleotide Synthesis: Methods and Applications”, Humana Press). Commercial services for oligonucleotide synthesis are also widely available

For modifying a target polypeptide by fusing the native polypeptide sequence to redox-regulated domains (for example those from SAL1), the underlying nucleotide sequences for both the native polypeptide and the engineered domains can be fused to generate a single, in-frame hybrid nucleotide sequence using conventional molecular biology techniques including restriction digest and ligation, recombination, and blunt-end ligation. See, for example, Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000). Additionally, the hybrid nucleotide sequence may also be generated by artificial gene synthesis via chemical means, as described earlier. For in vivo applications of the invention, the nucleotide sequences may be generated by the methods above to additionally include nucleotide sequences encoding transit and/or signal peptides for translocation into the correct cellular compartment and/or target cell. For in vitro applications, additional sequences may also be included, such as those encoding protein fusion partners (e.g. ubiquitin, MBP), protein purification tags (e.g. polyhistidine, glutathione S-transferase) and/or protease cleavage sites (ubiquitin, TEV protease, enterokinase, thrombin) for removal of fusion partners and purification tags post-purification using conventional heterologous protein expression and purification methods.

For small polypeptides, modifications may also be introduced into the target polypeptides through peptide synthesis. That is, the entire peptide sequence containing the modified residues may be generated via chemical synthesis. Such procedures are well established, for examples see Artherton and Sheppard (1989) “Solid Phase Peptide Synthesis: A practical approach” Oxford, England: IRL Press ISBN 0-19-963067-4 and Nilsson et al (2005) Annu Rev. Biophys. Biomol Struct. 34:91-118. Commercial services for peptide synthesis are also widely available.

Using the modified nucleotides, vectors, and methods of transforming said vectors into host organisms as outlined below, the modified polypeptides can be expressed in the target organism for in vivo applications. Alternatively the polypeptide may be heterologously expressed in vitro in bacterial, yeast, insect, plant and/or mammalian cells using conventional expression techniques prior to purification for other applications.

Modifications in nucleotide sequences encoding a desired polypeptide may be introduced into the desired nucleotide sequence by any appropriate methods as are known in the art. For example, single or multiple nucleotide insertions, deletions or substitutions may be introduced via recombination of the target mutation site with an introduced targeting nucleotide sequence. Such an introduced nucleotide sequence may, for example, comprise a nucleotide sequence to be introduced into the genome flanked either side by nucleotide sequences homologous to target sequences contiguous in or located either side of a desired mutation insertion point. In accordance with the methods of the present invention, a nucleotide sequence to be introduced into the genome may also include a selectable marker operably linked to desired regulatory regions (which may include, for example, a stress-inducible promoter).

The nucleotide sequences homologous to the target sequences may be isogenic with the target sequences to thereby promote the frequency of homologous recombination.

Homologous nucleotide sequences that are not strictly isogenic to the target sequences can also be used. Although mismatches between the homologous nucleotide sequences and the target sequences can adversely affect the frequency of homologous recombination, isogenicity is not strictly required and substantial homology may be sufficient. For the purposes of the present invention, the level of homology between the homologous sequences and the target sequences may be at least about 90% identity, at least about 95% identity, at least about 99% identity or 100% identity.

A targeting nucleotide sequence can be comprised in a vector. Representative vectors include plasmids, cosmids, and viral vectors. Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Selection of these and other common vector elements are conventional and many such sequences can be derived from commercially available vectors. See, for example, Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000).

A targeting vector can be introduced into targeting cells using any suitable method known in the art for introducing DNA into cells, including but not limited to microinjection, electroporation, calcium phosphate precipitation, liposome-mediated delivery, viral infection, protoplast fusion, and particle-mediated uptake.

Optionally, a targeting DNA is co-administered with a recombinase, for example recA, to a target cell to thereby enhance the rate of homologous recombination. The target cell(s) may already comprise, or have been transformed to comprise suitable recombinase target sequences, if required. For example, a recombinase protein(s) can be loaded onto a targeting DNA as described in U.S. Pat. No. 6,255,113. To enhance the loading process, a targeting DNA can contain one or more recombinogenic nucleation sequences. A targeting DNA can also be coated with a recombinase protein by pre-incubating the targeting polynucleotide with a recombinase, whereby the recombinase is non-covalently bound to the polynucleotide. See, for example, A. Vergunst et al (1998), Nucleic Acids Res. 26:2729 and A. Vergunst and P. Hooykaas (1998), Plant Molec. Biol. 38:393 406, International patent publications WO 99/25821, WO 99/25840, WO 99/25855, and WO 99/25854 and U.S. Pat. Nos. 5,780,296, 6,255,113, and 6,686,515.

Mutations may also be introduced into target organisms using zinc finger nucleases (ZFNs), transcription activator-like effector nucleases (TALENs), clustered regulatory interspaced short palindromic repeat (CRISPR)/Cas-based RNA-guided DNA endonucleases, and homing endonucleases (HEs) as discussed in, for example, Gaj T. et al (2013); Trends Biotechnol. 31(7): 397-405), Carroll D. (2012; Molecular therapy 20(9): 1659-1660), Xiao A. et al (6 Jun. 2013); Nucleic Acids Research 2013, 1-11, doi: 10.1093/nar/gkt464, the disclosures of these references being hereby incorporated by cross-reference.

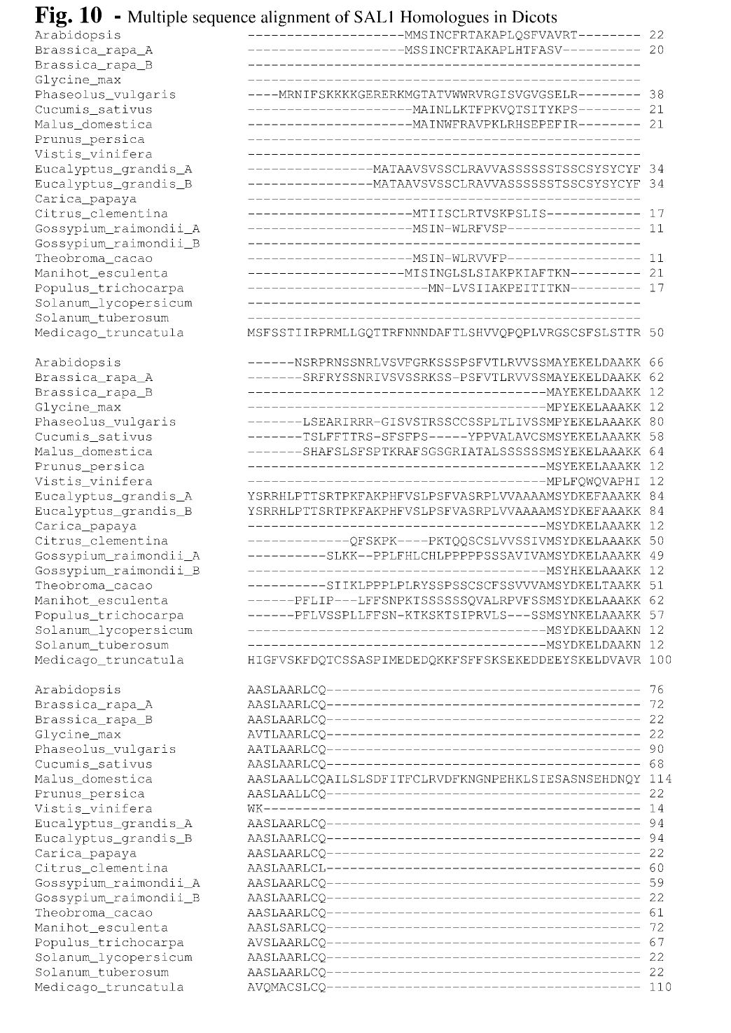

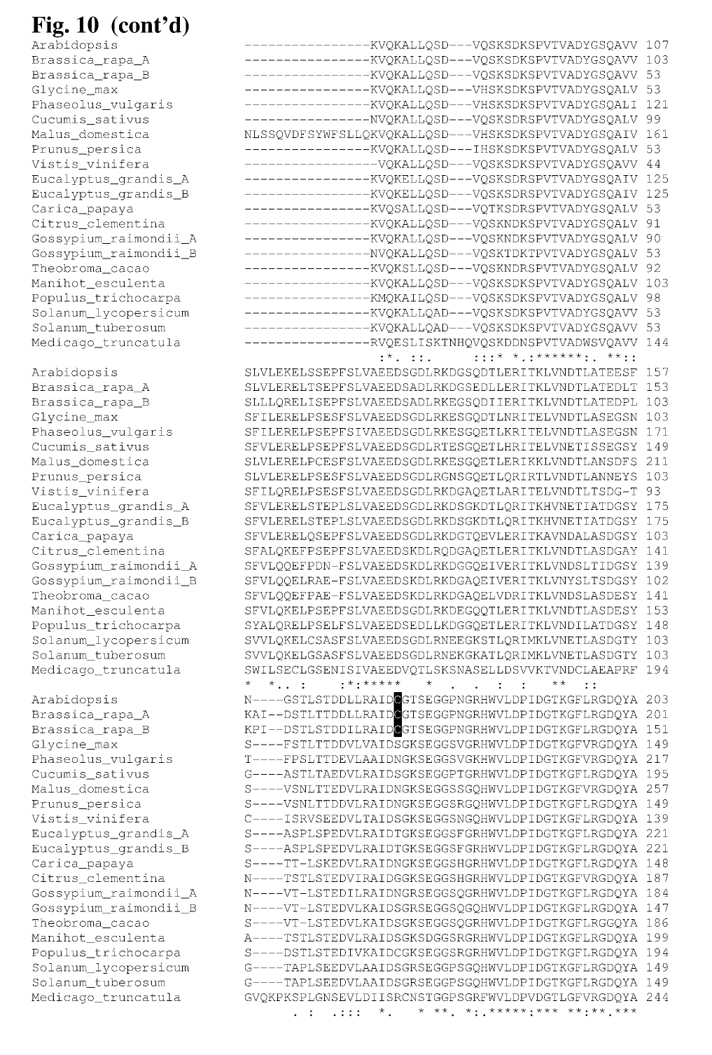

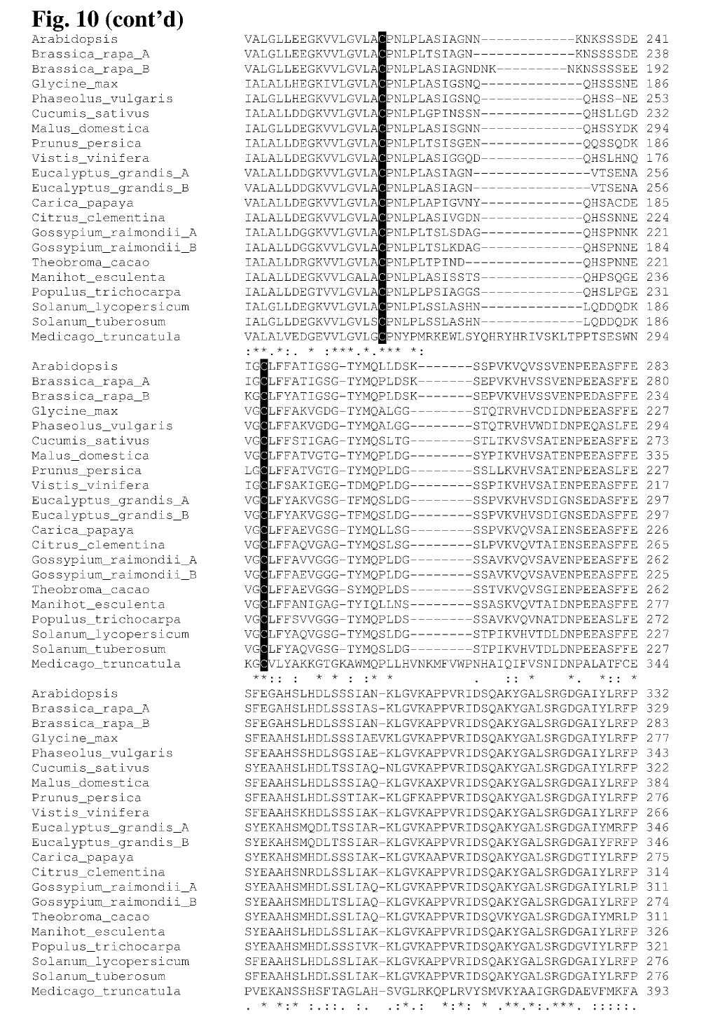

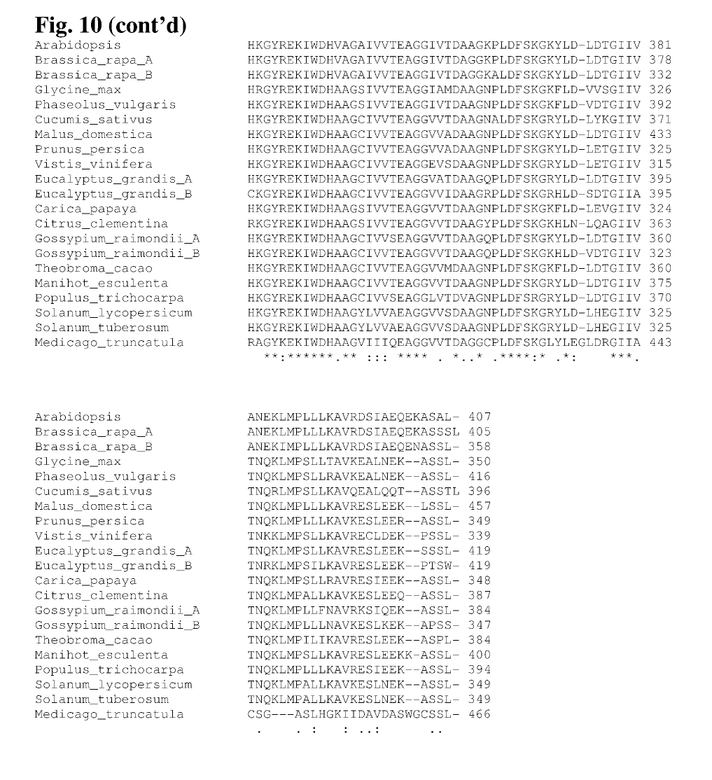

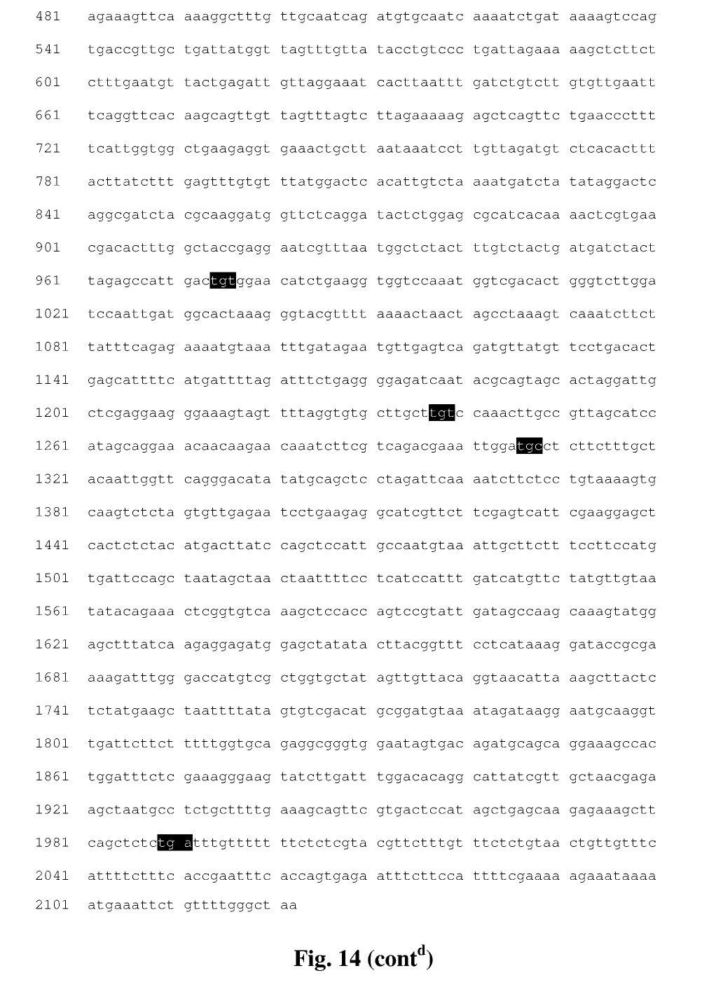

The present invention also contemplates altering the activity of a polypeptide or other compound by fusing/conjugating the polypeptide or compound to polypeptide domains/folds which are known to be redox sensitive (for example those from SAL1). Such fusing/conjugating may be carried out by any appropriate method as known in the art. The resulting fusion product or conjugate may have activity under reducing conditions (while the polypepetide domain(s)/fold(s) are intact), and then be inactivated in oxidising condition, or it may be inactive under reducing conditions, its activity being blocked or hindered by association with the intact polypeptide domain(s)/fold(s) but be activated by oxidising conditions.

Where the polypeptide already has two cysteines in close proximity and is redox sensitive, the polypeptide may be made more unstable under oxidising conditions by inserting known domains (e.g. those from SAL1) into their structure, by methods as known in the art.

Where the polypeptide is SAL1, or an analogue or homologue thereof, a method of the present invention may comprise substituting or deleting one or more amino acids as shown in SEQ ID NO:1 (FIG. 13) having reference to alignment of homologues with SEQ ID NO:1 (see, for example, FIGS. 10 and 11), or deleting, substituting or inserting one or more nucleotides in a sequence corresponding with SEQ ID NO:2 (FIG. 14), having reference to alignment of homologues with SEQ ID NO:2 (see, for example, FIG. 12.)

The method may therefore comprise deleting or substituting at least one of the cysteines at positions 75, 173, 221 or 244 of SEQ ID NO: 1, or an equivalent thereof in an analogous or homologous sequence.

Alternatively, where the polypeptide is an analogue or homologue of SAL1 lacking a cysteine at a position equivalent to at least one of the cysteines at positions 75, 173, 221 or 244 of SEQ ID NO: 1 and which comprises modifying said analogue or homologue to comprise cysteines at at least positions 75 and 173, or at positions 221 and 244, or at positions 75, 173, 221 and 244.

The method may therefore comprise substituting, deleting or inserting at least one nucleotide in positions 251-253, 974-976, 1237-1239 or 1306-1308 of a nucleotide sequence analogous or homologous to SEQ ID NO:2.

Alternatively, where the polypeptide is an analogue or homologue of SAL1 lacking a cysteine at a position equivalent to at least one of the cysteines at positions 75, 173, 221 or 244 of SEQ ID NO: 1 and which comprises substituting, deleting or inserting at least one nucleotide in positions 251-253, 974-976, 1237-1239 or 1306-1308 of a nucleotide sequence analogous or homologous to SEQ ID NO:2 such that a polypeptide expressed by the modified nucleotide sequence comprises cysteines at at least positions 75 and 173, or at positions 221 and 244, or at positions 75, 173, 221 and 244.

According to an embodiment of the invention, mutant SAL1 proteins as found in the alx8, fry1-2, fry1-3, salk_02882, or hos2 Arabidopsis thaliana mutants are excluded from the scope all of the methods of the present invention. In particular, according to an embodiment of the invention, the following mutations in SAL1 proteins are not encompassed or contemplated:

a mutation in SEQ ID NO:2 resulting in a stop codon instead of a codon encoding a tryptophan residue at position 341 in SEQ ID NO:1;

a guanine to adenine mutation at position 736 of SEQ ID NO:2;

a mutation in SEQ ID NO:2 resulting in a glutamic acid to lysine substitution at position 126 of SEQ ID NO:1;

a 6.7 kb T-DNA insertion between the fifth and sixth exons at position 1518 of SEQ ID NO:2;

a T-DNA insertion between positions 734 and 735 of SEQ ID NO:2;

a T-DNA insertion replacing nucleotides 735-745 of SEQ ID NO:2;

a cytosine to thymine mutation at position 731 of SEQ ID NO:1;

a mutation in SEQ ID NO:2 resulting in an alanine to valine substitution at position 124 of SEQ ID NO:1;

a guanine to adenine mutation oat position 1226 of SEQ ID NO:2; and a mutation in SEQ ID NO:1 resulting in a glycine to aspartic acid substitution at position 217 of SEQ ID NO: 2.

According to another embodiment of the present invention, the Arabidopsis thaliana alx8, fry1-1, fry1-2, fry1-3, salk_02882, and hos2 mutants are excluded from the scope of mutants according to the present invention.

### Methods for Detecting Inserted Mutations

Screening an organism for the presence of at least one mutation associated with an at least partially modified SAL1 encoding sequence may comprise analysing DNA of the organism using at least one nucleic acid molecule suitable as a probe or primer which is capable of hybridising to the relevant sequence under stringent conditions. In a more specific method, the screening method may comprise the use of at least one oligonucleotide primer pair suitable for amplification of a region of the sequence, comprising a forward primer and a reverse primer to detect the presence or absence of a mutation in said region. The region may comprise, for example, a whole gene, or may comprise only a portion thereof.

DNA from a transformed organism may be extracted by a number of suitable methods known to those skilled in the art, such as are described in a wide range of well-known texts, including (but not limited to) Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000), incorporated herein by cross-reference. See also the methods described in Lukowitz, W., Gillmor, C. S. and Scheble, W-R. (2000) “Positional Cloning in Arabidopsis: Why It Feels Good to Have a Genome Initiative Working for You” Plant Physiology 123, 795-805, and references cited therein.

Once suitable DNA has been isolated, this may be analysed for the presence or absence of a mutation by any suitable method as known in the art, and which method/strategy is employed may depend on the specificity desired, and the availability of suitable sequences and/or enzymes for restriction fragment length polymorphism (RFLP) analysis. Suitable methods may involve detection of labelled hybridisation product(s) between a mutation-specific probe and at least a portion of the target sequence, more typically, by amplification of at least a portion of the target sequence using either a primer and suitable probe, or using a pair of primers (forward and reverse primers) for amplification of a specific portion of the target sequence, followed by either direct partial and/or complete sequencing of the amplified DNA, or RFLP analysis thereof.

The methods and reagents for use in a PCR amplification reaction are well known to those skilled in the art. Suitable protocols and reagents will largely depend on individual circumstances. Guidance may be obtained from a variety of sources, such as for example Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000), incorporated herein by cross-reference.

A person skilled in the art would readily appreciate that various parameters of the PCR reaction may be altered without affecting the ability to amplify the desired product.

For example the Mg²⁺ concentration and temperatures employed may be varied. Similarly, the amount of genomic DNA used as a template may also be varied depending on the amount of DNA available.

Other methods of analysis of the amplified DNA to determine the presence or absence of a mutation are well known to those skilled in the art. For instance, following digestion of the amplified DNA with a suitable restriction enzyme to detect a mutation in the target sequence, the DNA may be analysed by a range of suitable methods, including electrophoresis. Of particular use is agarose or polyacrylamide gel electrophoresis, a technique commonly used by those skilled in the art for separation of DNA fragments on the basis of size. The concentration of agarose or polyacrylamide in the gel in large part determines the resolution ability of the gel and the appropriate concentration of agarose or polyacrylamide will therefore depend on the size of the DNA fragments to be distinguished.

Detection and/or determination of the existence of a mutation in the target sequence may be aided by computer analysis using any appropriate software. Suitable software packages for comparison of determined nucleotide sequences are well known in the art and are readily available.

If the mutation is in an unknown sequence, the mutation may need to be identified, located and/or characterised before it can be traced/followed through generations. Suitable methods for identifying, locating and characterising unknown mutations are known to those in the art and are described in a number of well-known standard texts, such as Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000). See also Rossel, J. B., Cuttriss, A. and Pogson, B. J. “Identifying Photoprotection Mutants in Arabidopsis thaliana” in Methods in Molecular Biology 274: 287-299 (Carpentier, R. ed, Humana Press).

More recent methods for identifying mutant sequences include ‘Tilling’, high resolution melts (HRMs) and deep sequencing/high-throughput sequencing.

TILLING (Targeting Induced Local Lesions in Genomes) is a method in molecular biology that allows directed identification of mutations in a specific gene. The method combines a standard technique (for example, mutagenesis with a chemical mutagen such as ethyl methanesulfonate (EMS)) with a sensitive DNA screening-technique that identifies single base mutations (also called point mutations) in a target gene. The first paper describing TILLING in Arabidopsis (McCallum C M, Comai L, Greene E A, Henikoff S, “Targeted screening for induced mutations”, Nat Biotechnol. (2000) April; 18(4):455-7, hereby incorporated by cross-reference) used dHPLC HPLC to identify mutations. The method was made more high throughput by using the restriction enzyme Cel-I combined with a gel based system to identify mutations (Colbert T, Till B J, Tompa R, Reynolds S, Steine M N, Yeung A T, McCallum C M, Comai L, Henikoff S, “High-throughput screening for induced point mutations”, Plant Physiol. (2001) June; 126(2):480-4, also hereby incorporated by cross-reference). Other methods of mutation detection, such as resequencing DNA, have been combined for TILLING. TILLING has since been used as a reverse genetics method in other organisms such as zebrafish, corn, wheat, rice, soybean, tomato and lettuce. See also: McCallum C M, Comai L, Greene E A, Henikoff S. “Targeting induced local lesions in genomes (TILLING) for plant functional genomics” Plant Physiol. (2000) June; 123(2):439-42; Colbert T, Till B J, Tompa R, Reynolds S, Steine M N, Yeung A T, McCallum C M, Comai L, Henikoff S. High-throughput screening for induced point mutations”, Plant Physiol. (2001) June; 126(2):480-4; Draper B W, McCallum C M, Stout J L, Slade A J, Moens C B, “A high-throughput method for identifying N-ethyl-N-nitrosourea (ENU)-induced point mutations in zebrafish”, Methods Cell Biol. (2004); 77:91-112; and Slade A J, Fuerstenberg S I, Loeffler D, Steine M N, Facciotti D, “A reverse genetic, nontransgenic approach to wheat crop improvement by TILLING”, Nat Biotechnol. (2005) January; 23(1):75-81, also hereby incorporated by cross-reference.

HRM (High Resolution Melt) is a recent development that can greatly extend the utility of traditional DNA melting analysis by taking advantage of recent improvements in high resolution melt instrumentation and the development of double strand specific DNA (dsDNA) binding dyes that can be used at high enough concentrations to saturate all double stranded sites produced during PCR amplifications (see http://www.corbettlifescience.com/control.cfm?page=Introduction_4&bhcp=1), as well as: Dufresne S D, Belloni D R, Wells W A, Tsongalis G J, “BRCA1 and BRCA2 Mutation Screening using SmartCyclerII high-resolution melt curve analysis”, Arch Pathol Lab Med (2006) 130: 185-187; Graham R, Liew M, Meadows C, Lyon E, Wittwer C T, “Distinguishing different DNA heterozygotes by high resolution melting”, Clinical Chemistry (2005) 51: 1295-1298; Hermann M G, Durtschl J D, Bromley K, Wittwer C T, Voelkerding K V, “Amplicon DNA melting analysis for mutation scanning and genotyping: cross-platform comparison of instruments and dyes”, Clinical Chemistry (2006) 52: 494-503; Liew M, Pryor R, Palais R, Meadows C, Erali M, Lyon E, Wittwer C, “Genotyping of single nucleotide polymorphisms by high resolution melting of small amplicons”, Clinical Chemistry (2004) 50: 1156-1164; Margraf R L, Mao R, Highsmith W E, Holtegaard L M, Wittwer C T, “Mutation Scanning of the RET protooncogene using high resolution melting analysis”, Clinical Chemistry (2006) 52: 138-141; NGRL (Wessex) Reference Reagent Report January 2006, “Plasmid based generic mutation detection reference reagents; production and performance indicator field trial”; NGRL (Wessex) Reference Reagent Report January 2006. “Production and field trial evaluation of reference reagents for mutation screening of BRCA1, BRCA2, hMLH1 and MHS2”; NGRL (Wessex) Reference Reagent Report June 2006, “Mutation Scanning by High Resolution Melts: Evaluation of Rotor-Gene™ 6000 (Corbett Life Science), HR-1™ and 384 well LightScanner™ (Idaho Technology)”; Reed G H, Wittwer C T, “Sensitivity and specificity of single-nucleotide polymorphism scanning by high resolution melting analysis”, Clinical Chemistry (2004) 50: 1748-1754; Willmore-Payne C, Holden J A, Tripp S, Layfield L J, “Human malignant melanoma: detection of BRAF- and c-kit-activating mutations by high-resolution amplicon melting analysis”, Human Pathology (2005) 36: 486-493; Wittwer C T, Reed G H, Gundry C N, Vandersteen J G, Pryor R J, “High-resolution genotyping by amplicon melting analysis using LCGreen” Clinical Chemistry (2003) 49: 853-860; Worm J, Aggerholm A, Guldberg P, “In-tube DNA methylation profiling by fluorescence melting curve analysis” Clinical Chemistry (2001) 47: 1183-1189; Zhou L, Myers A N, Vandersteen J G, Wang L, Wittwer C T, “Closed-tube genotyping with unlabeled oligonucleotide probes and a saturating DNA dye”, Clinical Chemistry (2004) 50: 1328-1335; and Zhou L, Wang L, Palais R, Pryor R, Wittwer C T, “High-resolution DNA melting analysis for simultaneous mutation scanning and genotyping in solution”, Clinical Chemistry (2005) 51: 1770-1777.

Oligonucleotide primers can be designed or other techniques can be applied to screen lines for mutations/insertions. Through breeding, a plant line may then be developed that is homozygous for a mutated copy of the target nucleotide sequence. PCR primers for this purpose may be designed so that a large portion of the coding sequence of the target nucleotide sequence is specifically amplified using the sequence of the target nucleotide sequence from the species to be probed (see, for example, Baumann, E. et al. (1998), “Successful PCR-based reverse genetic screens using an En-1-mutagenised Arabidopsis thaliana population generated via single-seed descent”, Theor. Appl. Genet. 97:729 734).

DNA constructs for transforming a selected plant may comprise a coding sequence of interest operably linked to appropriate 5′ regulatory sequences (e.g., promoters and translational regulatory sequences) and 3′ regulatory sequences (e.g., terminators). In a preferred embodiment, the coding region is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine synthase (OCS) promoters.

### Modulation of Seed Dormancy

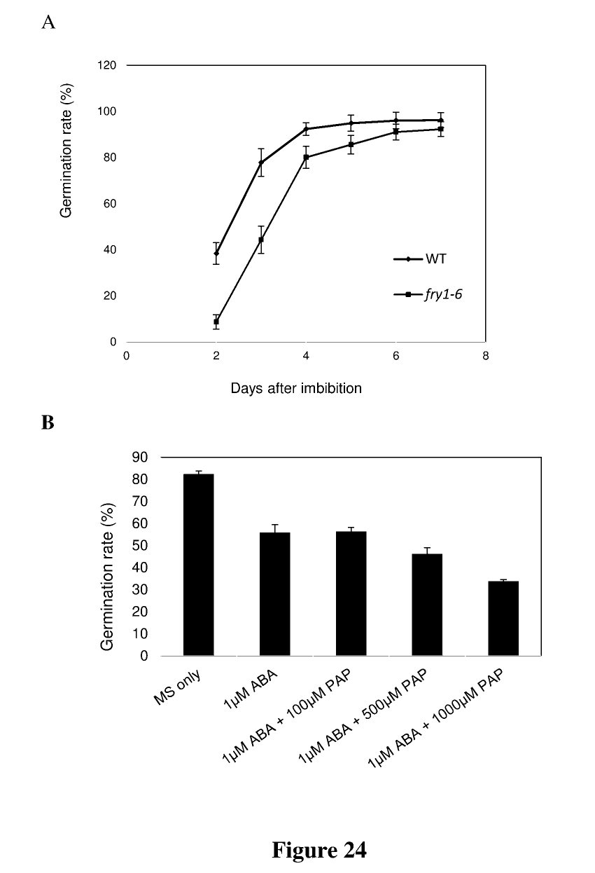

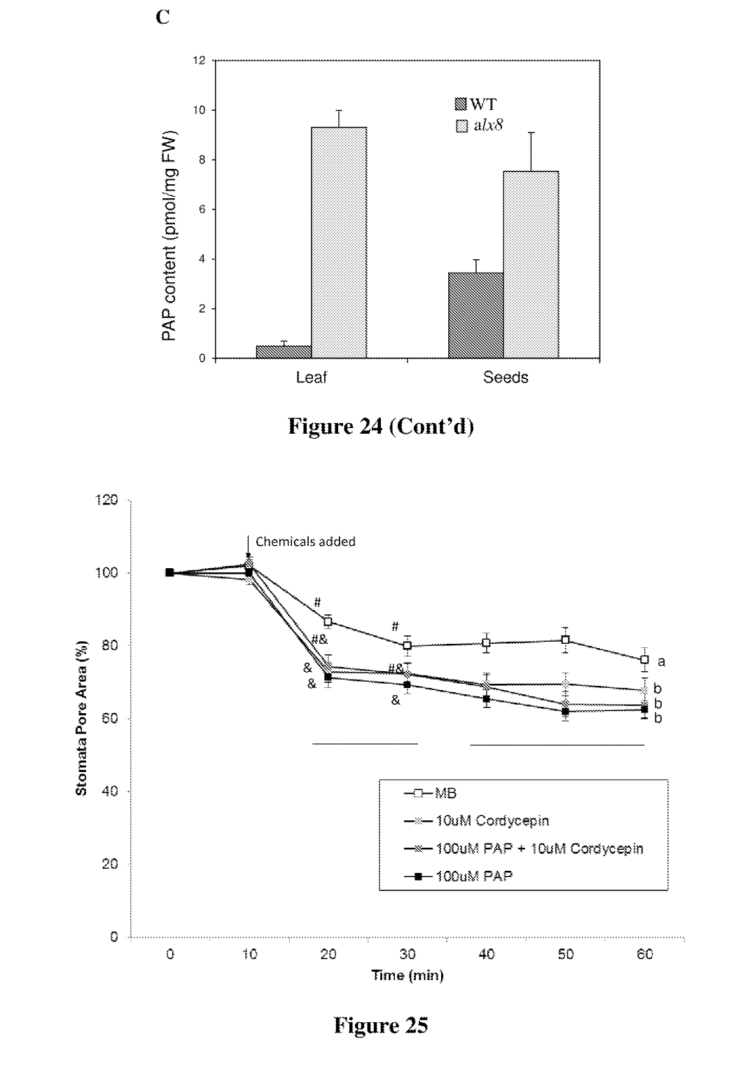

During the course of these studies, it was also noticed that seeds of plants that accumulated greater levels of PAP, also had delayed germination (and therefore extended dormancy) (see FIG. 24). Treatment of seeds with exogenous PAP extended the dormancy of seeds. Thus, an embodiment of the invention comprises treating seed with PAP or a derivative or analogue thereof.

It is contemplated that plants that accumulate PAP, through any of the treatments described above, will also accumulate PAP or a derivative or analogue thereof in their seeds.

Thus, in an embodiment, the present invention provides a method for extending the dormancy of a seed, as compared to an untreated seed, said method comprising accumulating in cells within said seed an increased level of PAP or a derivative or analogue thereof compared to an untreated or wild-type seed.

The seed may be obtained from a plant treated by a method according to the invention as described above.

Plants with Increased Stress Tolerance

Plants having modulated growth, compared to the plant(s) from which they are derived, obtained by any of the methods described above, are also encompassed within the ambit of the present invention. Such plants may include, for example, plants with increased or decreased growth (ie. increased or decreased biomass and/or increased or decreased seed or fruit yield), accelerated or delayed growth, shorter or longer life-cycles, earlier or delayed maturation. According to an embodiment, the present invention provides plants which are at least partially insensitive to environmental stresses (especially nutrient limitation, sodium or salt stress, drought, etc.) and therefore grow faster under those conditions compared to the plant(s) from which plants according to the invention are derived.

Also encompassed are plant parts, including but not restricted to leaves, stems, roots, tubers, flowers, fruits and seeds obtained from such plants.

Preferred forms of the present invention will now be described, by way of example only, with reference to the following examples (with relevant portions of Examples 1 to 3 having been previously presented in WO 2013/104026, but reproduced herein by way of complete description), including comparative data, and which are not to be taken to be limiting to the scope or spirit of the invention in any way.

### EXAMPLES

### Example 1—Materials and Methods

### Plant Materials, Growth and Stress Conditions

Seeds were germinated in soil and kept at 4° C. for 3 days to synchronize germination. Seedlings were grown at 100-150 μmol photons m⁻² s⁻¹, 12 h photoperiod, 21-23° C. and 50-55% humidity, unless otherwise stated.

In some experiments, the alx8 mutant (Col-0 background) was crossed with abi1-1, ost1-2 in the Ler background in order to generate double mutants. Homozygous F2 plants were screened using derived cleavable amplified polymorphic sequence (dCAPS) markers to confirm the presence of individual mutations and sequenced.

The Col-0 Ler F1 hybrid was generated as a control and in most experiments wild type refers to the F1 hybrid; otherwise both parental genotypes were used.

The ost1-2 snrk2.2 snrk2.3 triple mutant was obtained from Prof. Sean Cutler (University of California, Riverside) and crossed to the SAL1 null allele fry1-6 to generate the quadruple mutant.

Drought stress treatment was performed as previously reported.

For other oxidative stress treatments, leaf disks were floated on 0.1% Tween and either exposed to 1000 μmol photons m⁻² s⁻¹ white light (High-light); or to 500 mM H₂O₂, 100 μM methyl viologen (superoxide generator), or untreated (Control) under standard light intensity for 4 hours.

Five- to six-week-old plants were used to prepare epidermal peels. ABA at a concentration of 20 μM in water was root fed to hydroponically grown plants or sprayed onto leaves using a Studio Series IS-875 with a 0.5 mm nozzle opening (Iwata). We investigated if PAP accumulation in the double mutants could affect the production of NO during ABA-induced stomatal closure by treating epidermal peels with the inhibitor of NO synthase N(G)-methyl-L-arginine (NMA). For the investigation of the effect of different chemicals on stomatal closure, 20 μM ABA (Sigma-Aldrich), 100 mM LiCl, or 1 mM ATP, 1 mM or 100 μM PAP (Sigma-Aldrich) alone or in different combinations were prepared in infiltration buffer (1 mM PIPES KOH pH 6.0, 1 mM Sodium citrate, 1 mM KCl, 15 mM Sucrose) modified from a cordycepin infiltration buffer.

The mature full length WT AtSAL1 cDNA was amplified from WT Arabidopsis leaf cDNA using proofreading polymerase and cloned into the pGEM 3zf(+) vector. Mutagenesis was then carried out using different combinations of primers containing point mutations to change the individual cysteine codons into alanine, with the QuikChange II Site-Directed Mutagenesis kit (Stratagene, USA) as per manufacturer's instructions. All mutations were confirmed by Sanger sequencing. The resulting mutated AtSAL1 sequences were then cloned into either pHUE or prSETb (Life Technologies, USA), re-sequenced to confirm presence of mutation(s), and recombinant proteins purified as described below. For introduction of additional cysteines into ScHAL2 to produce ScHAL2+3C, the mutated sequence was synthesized and directly cloned into prSETb (Life Technologies, USA).

Protein Purification from Biological Samples

For screening of transgenic lines expressing SAL1, total leaf protein was extracted as follows. Briefly, leaf discs (approx. 25 mg) were flash frozen in liquid nitrogen, disrupted in Tissue Lyzer for 1 min at 30 Hz, and total protein extracted with 300 μl of extraction buffer [10% (v/v) glycerol, 150 mM Tris-HCl pH 8.0, 2% lithium dodecyl sulfate, 0.5 mM ethylenediaminetetraacetic acid (EDTA)] by vortexing and incubating on ice for 2 min. The sample was clarified by centrifugation at maximal speed on a table-top centrifuge at 4° C. and the clear-green supernatant mixed with approximately four volumes of −20° C. acetone. Total proteins were precipitated by incubation on dry ice for 45 min and pelleted by centrifugation for 10 min as before. A second acetone-wash of the pellet was performed to clear pigments and pellet disrupted by sonication. Precipitation was repeated as before, the pellet was dried in vacuum spin for 5 min and resuspended in 100 μl of solubilisation buffer (9 M urea, 4% (w/v) CHAPS, 1% (w/v) DTT, 35 mM Tris base). Western blot was performed for SAL1 as before and against PEPC using a 1:10000 dilution of polyclonal antibodies against maize recombinant PEPCase (kindly provided by Dr. Tsuyoshi Furumoto) as a loading control.

Arabidopsis leaf native proteins were extracted. Approximately 100 mg of either control or stressed Arabidopsis leaves were frozen in liquid nitrogen and ground to a fine powder with a ⅛″ steel ball with the TissueLyzer II (Qiagen, Germany). Native proteins were resuspended in ice-cold 50 mM Tris-HCl pH 7.5 supplemented with 1% PVP 360 and 1× Roche Protease Inhibitor Cocktail (Roche, Switzerland). Cellular debris was removed by centrifugation at 4° C. and the proteins in the supernatant quantified by Bradford assay. Native proteins were kept on ice and used immediately in Clear-Native PAGE and the activity assay, as described in later sections.

For detection of the C167-C190 intramolecular disulfide in endogenous AtSAL1, the native proteins were incubated with 10 mM iodoacetamide in the dark for 1 h to prevent oxidation of any cysteines that were reduced in vivo. Leaf protein was then precipitated in TCA/acetone, washed twice with cold acetone and resuspended in solubilisation buffer (9 M urea, 4% (w/v) CHAPS, 1% (w/v) DTT, 35 mM Tris base) before SDS-PAGE and western blotting as described below.

### Recombinant Protein Purification

Recombinant WT AtSAL1 protein was expressed in Escherichia coli BL21 DE3 cells (New England BioLabs, USA) as follows. The mature full length AtSAL1 cDNA (excluding the transit peptide sequence) was expressed as an AtSAL1-Ubiquitin fusion protein in the pHUE expression vector under IPTG induction and purified using Ni-NTA His-Bind Resin (Novagen, USA) according to manufacturer's instructions. AtSAL1-Ubiquitin was then digested with the deubiquitinylating enzyme Usp2 in a digestion buffer [50 mM Tris-HCl pH 8.0, 150 mM NaCl, 20 mM KCl, 2 mM β-mercaptoethanol] and re-purified through the Ni-NTA resin to yield the mature AtSAL1 protein of 95% purity as assayed by SDS-PAGE. The mature AtSAL1 protein was further purified into monomeric and dimeric fractions by size-exclusion chromatography (SEC) on a HiLoad 26/60 Superdex-200 SEC column (Life Technologies, USA). The monomeric (eluting between 195-220 mL) and dimeric (eluting between 170-195 mL) fractions obtained were concentrated by filter centrifugation, quantified by Bradford assay and stored in storage buffer [50 mM Tris-HCl pH 8.0, 150 mM NaCl, 20 mM KCl, 1 mM MgCl₂, 15% glycerol] at −80° C. Mutagenized variants of AtSAL1, WT ScHAL2, ScHAL2+3C and OsSAL1 proteins were purified essentially as described above, but were expressed with an N-terminus TEV protease recognition sequence in prSETb (Life Technologies, USA) instead of pHUE and the fusion protein was digested with TEV protease instead of Usp2 to yield the mature protein. WT Human BPNT1 was expressed in pNIC28 and purified as above.

### Protein Gel Electrophoresis and Immunoblotting

Recombinant protein, 0.5-1 μg, was incubated in degassed storage buffer [50 mM Tris-HCl pH 8.0, 150 mM NaCl, 50 mM KCl, 1 mM MgCl₂, 15% glycerol] in the presence of either 5 mM DTT (reducing conditions) or 5 mM oxidized DTT (oxidizing conditions) for 1 hour at RT, then NuPAGE LDS 4× Sample Loading Buffer (Life Technologies, USA) was added to a final concentration of 1×. Samples were heated at 70° C. for 10 minutes, cooled on ice and then resolved on a 10% Bis-Tris NuPAGE gel with MOPS buffer (Life Technologies, USA) at 150 V for 2.5 hours without addition of reducing agents or antioxidants. The protein bands were visualized by staining with GelCode Blue Safe Stain (Thermo Scientific, USA) according to manufacturer's instructions. For Clear-Native PAGE, proteins were incubated with redox agents as above but resuspended in Native Sample Loading buffer [100 mM Tris-HCl, 10% glycerol, 0.0025 bromophenol blue, pH 8.6) and resolved on a 3-12% Novex NativePAGE gel (Life Technologies, USA) in Native Running Buffer [25 mM Tris, 192 mM Glycine, pH 8.3] without denaturing agents.

For immunoblotting, total protein from tissue and cell fractions was extracted in 10% (w/v) tricarboxylic acid in cold acetone. 5 μg of leaf total protein extract and 5 ng of recombinant SAL1 (rSAL1) used as a positive antibody specificity control were resolved on 4-12% (w/v) SDS-PAGE (NuPAGE, Invitrogen), electro transferred to a nitrocellulose membrane and probed with a 1:1000 dilution of polyclonal antibodies raised against rSAL1 for 10 min. After 3 washes with PBS, the blot was incubated with 1:10000 dilution of HRP-conjugated goat anti-rabbit IgG for 10 min, washed 3 times and developed using the Super Signal® West FemtoChemiluminescent detection kit (Pierce) for 5 min.

### Crystallization, Data Collection and Refinement

AtSAL1 crystals were grown by vapor-diffusion in hanging-drops. Crystals formed at a protein concentration of 20 mg/ml in 20-30% PEG 2000-MME, 0.2M (NH₄)₂SO₄, 0.1 M HEPES pH 8.0-8.6. Diffraction data were collected at the Australian Synchrotron at the MX2 beamline (λ=0.9537 Å). The resolutions limits of the data were assessed on the basis of the significance of the CC_{1/2} at the P=0.001 level. Diffraction data were integrated using XDS and scaled using SCALA from the CCP4 program suite. The crystals belonged to the P6₁ space group and were merohedraly twinned (twin fraction: 0.508 for H, K, L and 0.492 for K, H, −L). Phases were obtained by molecular replacement in Phaser using the yeast homolog of SAL1 (PDB 1QGX) as the search model. The crystallographic asymmetric unit contained two copies of SAL1. Amplitude based twin-refinement was completed in Refmac5.

### Amino Acid Alignment

SAL1 amino acid sequences from the plant kingdom were retrieved from the Phytozome database. Amino acid sequences of SAL1 homologs in plants were aligned using the EBI web tool Clustal Omega program with default parameters, and visualized in GeneDoc.

### Estimation of Endogenous SAL1 Activity in Leaves

Arabidopsis leaf native proteins were extracted as follows. Approximately 100 mg of either control or ABA-treated Arabidopsis leaves were frozen in liquid nitrogen and ground to a fine powder with a ⅛″ steel ball with the TissueLyzer II (Qiagen, Germany). Native proteins were resuspended in ice-cold 50 mM Tris-HCl pH 7.5 supplemented with 1% PVP 360 and 1× Roche Protease Inhibitor Cocktail (Roche, Switzerland). Cellular debris was removed by centrifugation at 4° C. and the proteins in the supernatant quantified by Bradford assay. Native proteins were kept on ice and used immediately in activity assays, where 10 μg of total native protein extract was incubated with three different concentrations of PAP in Activity Buffer (100 mM Tris-MES pH 7.5, 1 mM Mg acetate) and initial activity assayed at 25° C. The reaction was stopped by flash-freezing in liquid nitrogen, and AMP produced by SAL1 degradation of PAP was quantified using the method for derivatization and detection of adenosines (see below).

### Activity Assays

For Michaelis-Menten kinetics, the activity of recombinant protein against PAP was assayed by incubating 0.2 μg protein in degassed Activity Buffer [100 mM Tris-MES pH 7.5, 1 mM Mg acetate] in the presence of either 5 mM DTT or 5 mM oxidized DTT (trans-4,5-Dihydroxy-1,2-dithiane) for 1 hour at 25° C., then increasing concentrations of PAP was added to a final volume of 150 μL and initial activity assayed at 25° C. The reaction was stopped by flash-freezing in liquid nitrogen, and AMP produced by SAL1 degradation of PAP was quantified using the method for derivatization and detection of adenosines via High Performance Liquid Chromatography. All Michaelis-Menten kinetics parameters were calculated using GraphPad Prism (GraphPad Software Inc., USA). For redox titration of activity, SAL1 protein was incubated as above with different ratios of reduced to oxidized DTT (final total concentration of 5 mM) or reduced to oxidized glutathione (GSH: GSSG, final concentration of 20 mM). Values for redox midpoint potential, E, was calculated by fitting titration data to the Nernst equation by using GraphPad Prism:

Eₕ=Eₘ+(RT/nF)(ln([GSSG]/[GSH]²)) for glutathione

And

Eₕ=Eₘ+(RT/nF)(ln([oxidized DTT]/[reduced DTT]))

with an RT/F of 25.7 mV and n=2.

For activity of AtSAL1 in native protein extracts from Arabidopsis, 10 μg of total native protein extract was incubated in the same Activity Buffer as above with increasing concentrations of PAP at 25° C. without any redox agents.

For estimation of inhibition of AtSAL1 activity by PAP analogue 12, 200 ng of purified recombinant SAL1 protein was incubated in 75 μl 2×activity buffer (200 mM Tris-MES pH 7.5, 2 mM Mg acetate) and Analogue 12 at a concentration of 0, 3.35, 13.4 or 50 μM for 1 hour at room temperature. The samples were then placed on ice, and 75 μl PAP added for a final reaction condition of 100 mM Tris-MES pH 7.5, 1 mM Mg acetate and 50 μM PAP. The reaction was allowed to proceed at 25 degrees Celsius for 5 minutes, then stopped by flash freezing in liquid nitrogen. The AMP formed from degradation of PAP by SAL1 was quantified by derivatization and resolving on HPLC. Duplicate treatments were carried out, and data presented as mean+/−standard error.

### Quantification of Metabolites

Total adenosines were extracted with 0.1 M HCl, derivatization with chloroacetaldehyde and quantified fluorometrically after HPLC fractionation. 3′-phosphoadenosine 5′-phosphate (PAP) quantification was performed by integrating the HPLC peak area and converting these to pmol units using standard curves of 1, 5 and 10 pmol standard.

Total leaf ABA content was quantified using a modified ELISA-based method. Approximately 100 mg of leaf tissue was harvested, immediately flash frozen with liquid nitrogen, and ground to a fine powder with a ⅛ ″ steel ball bearing in a 2 ml Eppendorf tube at 25 Hz for 2 min using the Tissue Lyser II (Qiagen, Germany). ABA was extracted from the ground tissue by shaking overnight (25 rpm, 4° C.) in 2 ml of 80% (v/v) methanol, followed by centrifugation (16000 g) at 4° C. The supernatant was collected, dried under vacuum to 25% of its original volume, and acidified to pH 3.0 using 0.5 M HCl. An equal volume of ethyl acetate was then added and allowed to mix by gentle inversion for 10 s. After standing for 30 s the upper organic layer of ethyl acetate was transferred to another 2 ml Eppendorf tube and dried under vacuum to complete dryness. The resulting pellet was resuspended in 1 ml of 1× Tris-Buffered Saline (TBS) [3.03 g/L Trizma base, 5.84 g/L Sodium chloride, 0.2 g/L Magnesium chloride hexahydrate, 0.2 g/L Sodium azide, pH 7.4]. A 1:10 dilution in TBS of each sample was used for the quantification assay, which was performed using the Phytodetek ABA Quantification Kit (Agdia, USA) according to the manufacturer's instructions.

### Measurement of Accumulation of PAP Analogues in Leaves Post-Spraying

Five week-old soil-grown wild type Arabidopsis plants were sprayed using a Studio Series IS-875 with a 0.5 mm nozzle opening (Iwata). Each plant was sprayed with approximately 100 μl of different PAP analogues (100 mM) or mock buffer (1% DMSO) and then returned to standard growth conditions. 100 mg leaf tissue was harvested and snap-frozen in liquid nitrogen at various timepoints post-spraying (1 hr, 4 hr, 1 d and 4 d). Total adenosines were extracted, derivatized and quantified by HPLC. Identification and quantification of Analogues 8, 12 and 13 were performed by comparison to authentic standards. Triplicate treatments were carried out, and data presented as mean+/−standard deviation.

### Gas Exchange Measurements

Six-week-old hydroponically grown plants were transferred from nutrient solution to the nutrient solution containing 20 μM ABA (Sigma-Aldrich). The effect of ABA on stomatal conductance (gₛ) was measured after 2 h of incubation (3 leaves per plant) using a LI-COR LI-6400 (LI-COR, USA).

### Infra-Red Thermography

Leaf temperature was monitored over time using an infra-red (IR) camera FLIR A600-Series, IR lens f=13.1 mm (FLIR Systems AB, Sweden). The temperature of individual leaves was monitored using the ThermaCAM™ Researcher Pro 2.10 software (FLIR Systems AB) and tissue harvested for total RNA and adenosine extraction after 2 h of incubation. Leaf temperature measurements of plants treated with PAP analogues were performed using a PSI IR camera (Photon Systems Instruments, Czech Republic) and analyzed using RIA Analyzer (Photon Systems Instruments, Czech Republic).

### Stomatal Bioassays

Measurements of stomatal aperture were performed with epidermal strips taken from five- to six-week-old plants. Fully developed leaves were cut and placed with their adaxial side onto cello tape. The vein was removed by razor blade and the abaxial epidermis was subsequently peeled off by using another strip of cello tape. To promote stomatal opening, the peels left adhered to the Scotch (810D Magic Tape, ¾ inch, 3M) were placed in petri dishes filled with stomatal opening solution (10 mM MES, 5 mM KCl, 50 μM CaCl₂, pH 6.5) and incubated for 2 h at 22° C. under 150-200 μmol photons m⁻² s⁻¹ of light. Once the stomata were fully open, strips were maintained in the opening buffer or treated with various compounds according to the experiment. After treatment, photographs of stomata were taken using Leica microscope (Leica Microsystems, Wetzlar, Germany) at 20×. Stomatal aperture was measured using the image-processing software ImageJ. For stomatal aperture measurements, the ratio of stomatal pore width and length was used instead of width as this normalizes the pores of stomata of different size. Small stomata with a stomatal pore length of less than 7.5 μm were discarded. For each data point, 90 stomata were measured in 3 independent experiments. In each experiment, 10 stomata were measured in 3 epidermal strips per sample per treatment.

Measurements of stomatal closure in response to ABA, PAP, cordycepin and ATP treatments were performed on epidermal strips from Arabidopsis and barley plants grown under specific conditions. Plants were grown under high humidity (approximately 80% RH) and 12 h/12 h and 22/20° C. light/dark cycle in a growth chamber. Epidermal peels of newly expanding true leaves of three to four-week old plants. Stomatal apertures were measured in epidermal peels of newly expanded leaves. Peels were taken from the abaxial leaf surface and were fixed to the glass bottom of the experimental chamber after coating the chamber surface with an optically clear and pressure-sensitive silicon adhesive (Factor II, Tucson, USA). The attached epidermal peel is subsequently immersed in an opening buffer (OB: 50 mM KCl, 5 mM MES titrated to pH 6.1 with NaOH) and incubated, at plant growth conditions, for approximately 2 h. Stomatal images were taken using a bright-field microscope capable of 400× magnification for 10 minutes in OB and subsequently in Measuring Buffer [MB: 10 mM KCl, 5 mM MES titrated to pH 6.1 with Ca(OH)₂] or PAP or cordycepin or ABA (dissolved in MB) for another 50 min. In total this was a 60 minute time course and images of the epidermal section were taken every 5 minutes using NIS Elements software (Nikon, Japan). Stomata aperture width and length were measured using Image J (NIH, USA). The pore area of each stomate was calculated using these values under the assumption that the area of a stomatal pore was that of an ellipse. Data are expressed as percentage over the control.

The ABA- and PAP-induced accumulation of ROS in guard cells of epidermal peels was measured using dichlorofluorescein diacetate (H2DCFDA, Invitrogen). After 2 h of incubation in OB, the epidermal peel was incubated with 20 μM dye dissolved in DMSO and MB for 30 min, and then rinsed with MB three times. Epidermal peels were incubated in ABA or PAP dissolved in MB (or MB only as a negative control) for 10 min. Fluorescence images were taken using a Leica inverted confocal microscope controlled by the LAS AF software (Leica Microsystems, Germany). We used the 496-nm excitation line of an argon multiline laser. H2DCFDA fluorescence emission was detected at 505 to 525 nm. Chloroplast fluorescence was detected at 680 to 700 nm in order to separate the autofluorescence of chlorophyll in guard cells. Images were quantified with Image J software (National Institutes of Health, USA). The background signal was measured from the empty region of the similar size and subtracted from the stomata signal to obtain corrected total stomatal fluorescence values. Each stomate was considered to represent a single biological unit and thus fluorescence values were averaged across all stomata at each time point for each treatment. At least three biological replicates were measured for each treatment.

### Effect of PAP and Analogues Thereof on Stomatal Aperture

Five week-old soil-grown wild type Arabidopsis plants were sprayed using a Studio Series IS-875 with a 0.5 mm nozzle opening (Iwata). Each plant was sprayed with approximately 100 μl of either 100 mM PAP, different PAP analogues or mock buffer (1% DMSO) and then returned to standard growth conditions. Leaf temperature was measured using an infrared camera before spraying and at four hours post-spraying. Raw data files were processed and analyzed using RIA Analyzer (PSI). Five replicates per treatment were carried out, and data presented as mean+/−standard error.

### Seed Germination Assays

Seeds used for germination assays were 3-months-old and all from seed stocks produced and harvested from plants grown under identical conditions at the same time. Seeds were sterilized and grown on 0.8% (w/v) agar plates containing Murashige-Skoog (MS) nutrients and 2% (w/v) sucrose. Different combinations of 0, 1 μM ABA, 0, 0.5 μM paclobutrazol and/or 0, 100, 500 and 1000 μM PAP were supplemented into the media using the infiltration buffer. At least 70-100 seeds from each genotype were plated on the same plate for efficient comparison. The seeds were stratified at 4° C. in the dark for 48 h before transfer to a growth cabinet and grown at 22° C. under 24-h light (100 μmol photons m⁻² s⁻¹) unless stated otherwise. The germination was scored after 3 days as the emergence of the radicle through the seed coat and the germination rate was calculated as the percentage of the total number of seeds per treatment with five replicates for each treatment. The experiment was repeated twice and statistical analyses are described below.

### Imaging

Plant photographs were taken using the Lumix DMC-FZ5 camera (Panasonic, Japan). Examination of Arabidopsis leaves was performed using Cambridge S360 (SEM; 1987; Leica/Cambridge, Wetzlar, Germany). All images were saved in TIFF format and analyzed using ImageJ software. The stomatal index (SI) was calculated as SI=100×[S/(E+S)], where S is the number of stomata and E is the number of epidermal cells. Leica microscope (Leica Microsystems GMBH, Wetzlar, Germany) was used to observe changes in stomatal aperture after various chemical treatments. All images were taken using SPOT Advanced software (SPOT™ Imaging Solutions, MI, USA) for Windows version 4.0.9.

### Global Transcript Analyses

Analysis of the changes in transcript abundance between Col-0Ler, alx8, ost1-2 and ost1-2 alx8 in the presence or absence of ABA was performed using Affymetrix Arabidopsis gene 1.0 ST arrays. Tissue samples were collected after 45 min of treatment for RNA analyses in biological triplicates using one leaf per sample per genotype per treatment. Total RNA was extracted using the guanidinium thiocyanate-phenol-chloroform extraction method using Trizol (Ambion), DNA removed using TURBO Dnase, RNA quality verified using a Bioanalyzer (Agilent Technologies), and spectrophotometric analysis was performed to determine the A260:A280 and A260:A230 ratios. Labeling and hybridization to Affymetrix Arabidopsis Gene 1.0 Arrays was performed by the Ramaciotti Centre for Genomics (University of New South Wales, Sydney, Australia).

Pre-processing and statistical analysis of the Affymetrix Arabidopsis Gene 1.0 ST Arrays was performed in R (v3.1.0) using the Bioconductor packages oligo (v1.28.0) and Limma (v3.20.1) respectively; and also using Affymetrix Power Tools suite (APT). Data quality checks and analysis was also performed using Partek Genomics Solution (PGS) software version 6.6 (default parameters; Partek, St. Louis) and near identical results were obtained compared to the R workflow. For R analyses raw cel files were read into R and the exon probe sets were background corrected, quantile normalized and expression values were summarized (using the median-polish) to the transcript level by calling the rma( ) function with the target=‘core’ option (to summarize to gene level features with the highest annotation confidence/evidence). Affymetrix Power Tools were employed to calculate the detection above background (DABG) probability for each probe set. A gene was considered present if at least one half of its constituent exons were assigned a detection P<0.05, only genes that were detected as present in greater than half the sample replicates in at least one sample group were retained for further analysis. Probe sets were further filtered to remove unannotated and control probe sets as well as any probe set reported to cross-hybridize (only probes annotated as crosshyb=1 by Affymetrix were retained), giving a final set of 13,780 detected genes.

### Statistical Analyses

For statistical analysis of differential gene expression, a linear model was fitted for the 2-factor design and coefficients determined for each of the 8 genotype x treatment factors (i.e. sample groups analyzed; equivalent to applying a classical genotype*treatment interaction model) using the lmFit function followed by empirical Bayes smoothing of the standard errors using eBays. Contrasts of interest were extracted and decideTests and topTable were applied to determine differentially expressed transcripts between the different conditions/genotypes using the adjust. method=“BH” option for the Benjamini-Hochberg (1995) method to adjust P-values for multiple comparisons. Transcripts were considered differentially expressed where the adjusted P-values were <0.05 (FDR 5%) and absolute fold-change >1.5. Gene-set enrichment tests were conducted using roast. For the gene-set analyses only probes mapping to a single TAIR10 gene were considered.

For biochemical and physiological experiments analyses of variance (ANOVA) were used to test for significant (p<0.05) differences between three or more sample groups for a particular treatment or time point. In limited cases where just two sample groups of interest were compared, the two-sample student's t-test assuming equal variance was used. Statistical procedures were carried out using the GraphPadInStat software (version 3.06).

For infrared thermography values over time after chemical spraying for individual replicates were fitted to orthogonal polynomial equations (two pots per treatment, two to three plants per pot). The calculated coefficients for each replicate were statistically compared to each other using ANOVA. Overall, there were significant differences. First, whole data set: F pr value per treatment (t8 F pr)=<0.001, 0.02, 0.003, <0.001 for variate constant, linear, quadratic or cubic, respectively; s.e.d values varied for each variate and were used to estimate the least square difference (LSD) as ˜s.e.d t×8 F pr). Second, reduced data set: F pr value per treatment=<0.001, 0.02, 0.01, 0.008 for variant constant, linear, quadratic or cubic, respectively.

Statistical analysis on seed germination experiments were performed using ANOVA (3- or 4-way) followed by Tukey's Honestly Significant Difference (HSD) post-hoc test in R software version 3.0.2.

For stomatal aperture R (version 3.1.3) was used for statistical analyses, performing an ANOVA to test for significant differences (p<0.05) in relative pore area between treatments at each time point using the aov function, as part of the R ‘stats’ package, and modelling stomatal closure (X) with a nested model (timepoint nested within treatment: X ˜Treatment/Timepoint). Additionally, to look at rates of closure, a mixed-effect model was produced taking into account random effects between stomates nested with peels: X ˜Timepoint*Treatment+Peel.ID/Stomate.ID. This model observed only timepoints 10-25 minutes as this was considered to be the predominant period of stomata closure. This was conducted using the lmer function from the lme4 package (used alongside the lmerTest package which influences the tests performed by the lmer function). Importantly, for these analyses, each stomate was considered to represent a single biological unit. This assumption was confirmed from the mixed effect model produced, which demonstrated that the variation that occurred between stomata from a single epidermal peel (0.1779) was less than the variation observed between epidermal peels (0.2490) for a particular treatment.

### Example 2—Previous Studies

As previously reported (International patent publication no. WO 2008/154695; Rossel J. B. et al (2007), Plant Cell 19(12):4091-4110 and in Wilson P. B. et al (2009), Plant Journal 58(2): 299-317), an Arabidopsis thaliana mutant, alx8 comprised a mutation in the gene encoding SAL1, that reduced expression of this protein. This plant, as well as other Arabidopsis thaliana plants comprising a mutation in this gene and deficient in SAL1 expression, were found to grow substantially normally under well-watered (WW), and normal to low light (LL) conditions (FIGS. 1A, 1C). However, under high light (HL) stress and water-stressed (WS) conditions, the sal1 mutants were found to accumulate less reactive oxidation species (ROS) (FIG. 1B) and to tolerate water-stressed conditions better (FIG. 1C) than wild-type plants.

Previous studies (Estavillo G. M. et al (2011), Plant Cell 23(11): 3992-4012) also showed that PAP is the substrate for SAL1, rather than PAPS or IPS as previously suggested. A proposed role for SAL1 in sulfur assimilation reactions is shown in FIG. 2A. These findings are supported by metabolite studies in sal1 mutants, that showed about 20-fold PAP accumulation, with small or insignificant changes in PAPS and inositol metabolite pools (FIGS. 2B, 2C).

The correlation between higher PAP levels in alx8 and drought tolerance led us to investigate a role for this metabolite during abiotic stress responses. Thus, we analyzed PAP levels in response to drought and HL in wild-type plants. PAP levels increased 30-fold in leaves of drought-stressed wild-type plants, coincident with a substantial decrease in plant relative water content (RWC) after 7 to 11 d of drought (FIG. 3). Analysis of variance (ANOVA) two-factor analyses indicated strong interaction between day and genotype for RWC and PAP, being significantly higher for alx8 relative to Col-0 (P<0.001). This increase did not occur in the early phase of drought and was observed only when there was a decline in RWC. A similar trend was observed for alx8, but it was delayed, again with PAP only rising as RWC declined. Similarly, exposure of Col-0 plants to HL for just 1 h resulted in significantly (P<0.005) higher PAP levels than in plants kept at LL (0.9 6 0.2 versus 0.6 6 0.2 pmol of PAP/mg fresh weight [FW], respectively), although this increase was much smaller than that observed during drought. Taken together, these results reveal that the level of the sulfur-related metabolite PAP is elevated in mutants lacking SAL1 and increased in response to at least two abiotic stresses.

Two studies have reported on the complementation of the morphological phenotypes of sal1 mutants by targeting SAL1 to the nucleus and Sc-SAL1 to the chloroplast. However, the significance of these findings with respect to PAP acting as a retrograde signal was not considered in those reports; rather, they concluded that the constructs used demonstrated the location of SAL1. PAP levels, chloroplast-specific responses, and drought responses were not measured. Furthermore, as mentioned above, the differing reported localizations of SAL1 have prevented any systematic analysis.

Our studies show that total leaf PAP pools can be significantly lowered by targeting Sc-SAL1 exclusively to the chloroplast and that induction of the nuclear gene APX2, which is routinely used to study HL and drought stress-induced retrograde signaling, was lowered when PAP was lowered by chloroplastic SAL1 complementation.

There are several lines of evidence that support the notion that PAP can move between cellular compartments, as shown in FIG. 4. First and most compelling is that nuclear targeting of SAL1 results in the full complementation of sal1 mutant phenotypes, including total leaf PAP levels, APX2 expression in LL and drought, ELIP2 expression in LL and HL, and drought tolerance. Second, sal1 and xrn2 xrn3 double mutants show a very similar molecular and morphological phenotype, suggesting that PAP accumulation can inhibit XRN function as originally proved in yeast and suggested to occur in plants. Indeed, it is reasonable to assume that once in the cytosol, PAP would diffuse freely through the nuclear pore as do other nucleotides. Thus, degradation of PAP pools in either the chloroplast, mitochondria, or nucleus have the potential to restore the wild-type phenotype at the molecular level. This could be interpreted as PAP being able to move between cellular compartments.

### Example 3—ABA and PAP Accumulate During Drought Stress and Regulate Stomatal Closure

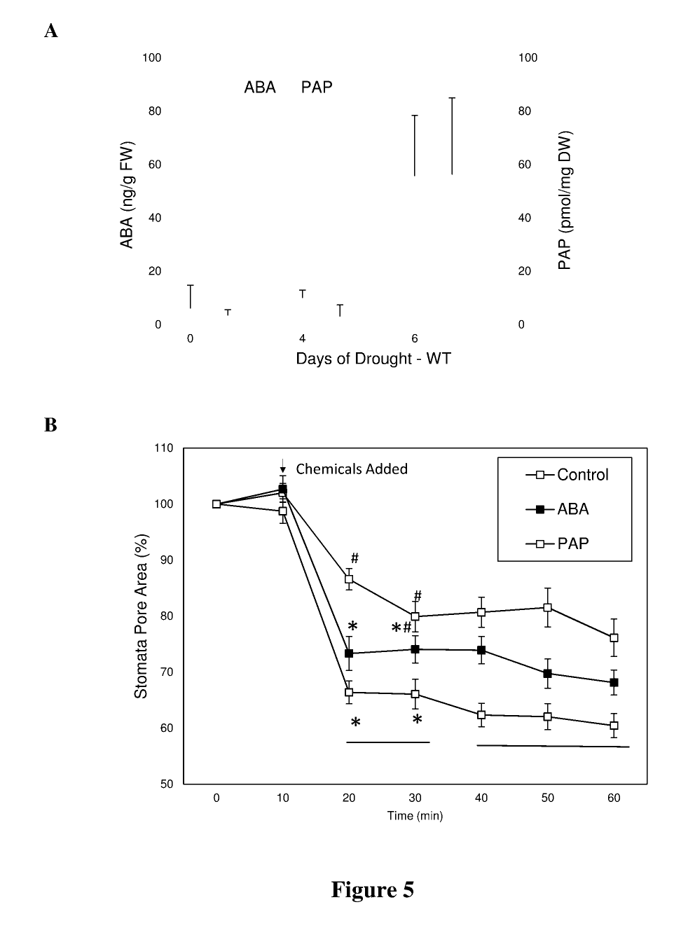

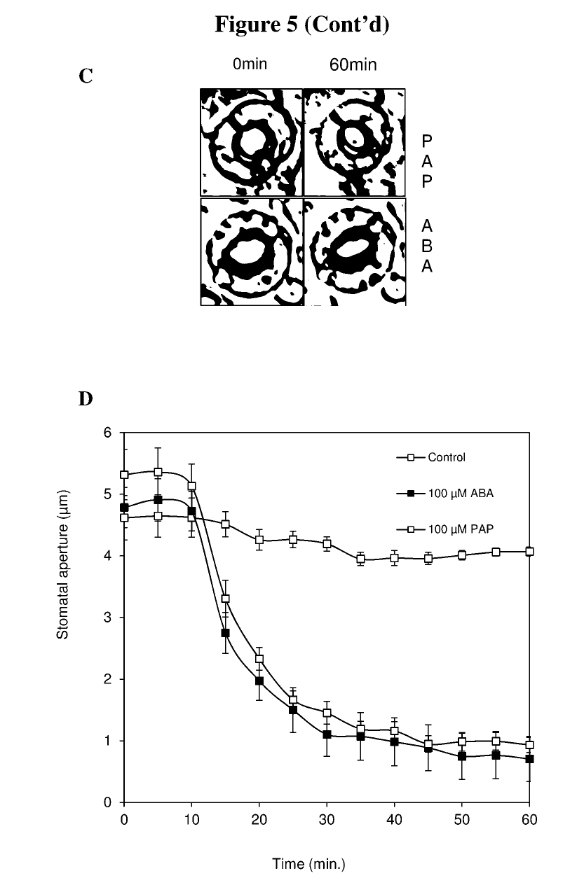

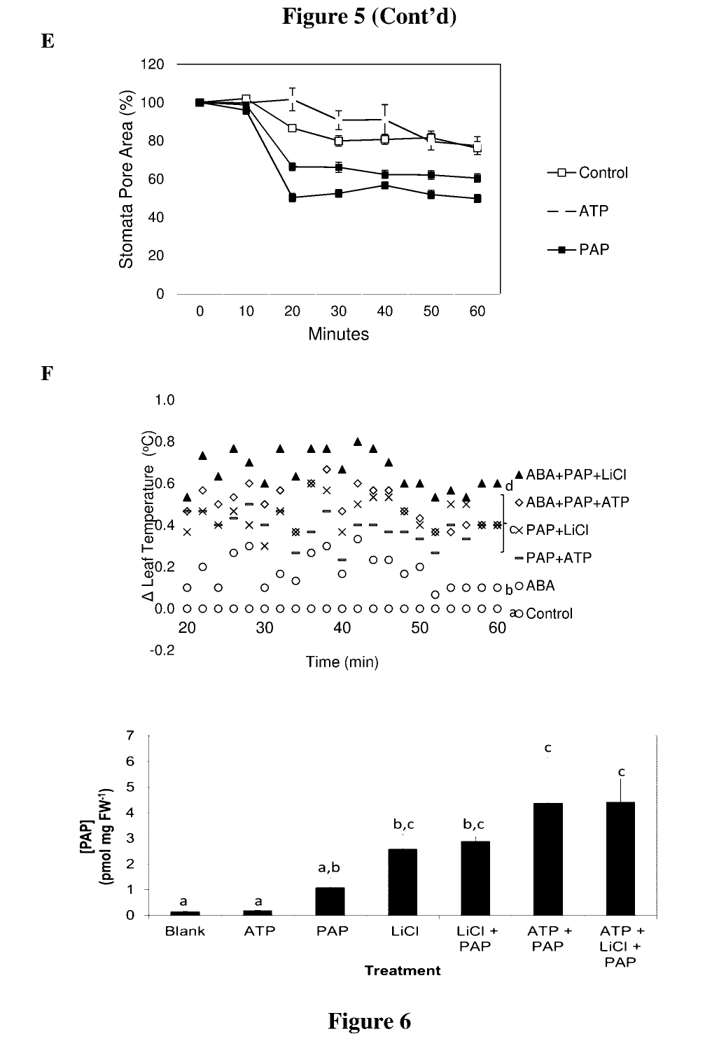

Our studies reveal that the two metabolites, ABA and PAP, accumulate concurrently under drought conditions (FIG. 5A). Exogenous ABA stimulates PAP accumulation by four fold, reaching levels similar to those observed in 6 days water stressed wild type leaves. This ABA-mediated PAP accumulation was not due to a change in SAL1 activity, but may have proceeded via stimulation of PAP synthesis.

We hypothesized that the SAL1-PAP retrograde pathway may be directly involved in guard cell signaling. If this is the case the pathway should be present in guard cells and manipulation of it should alter stomatal dynamics. Indeed, in addition to the vasculature SAL1 is enriched in epidermal peels and localized to chloroplasts of guard cells. Second, treatment of leaf peels with PAP elicited rapid stomatal closure similar to the ABA response (FIGS. 5C and 5D). Moreover, a stronger, cumulative response was achieved when in vivo degradation of exogenous PAP was attenuated with LiCl, an inhibitor of the PAP catabolic enzyme SAL1, or when PAP transport into the chloroplast, the site for its degradation, was prevented by ATP, a known co-substrate for the PAP antiporter (FIGS. 6 and 5E). The magnitude and rapid rate of ABA-mediated closure was further enhanced by PAP. These results were also shown through thermographic results (with increased difference in leaf internal temperature and external temperature as a result of stomatal closure), as shown in FIG. 5F, the greatest stomatal closure being shown for ABA+PAP+LiCl and ABA+PAP+ATP. Therefore, ABA stimulates PAP accumulation, PAP enhances ABA sensitivity, yet, PAP in and of itself can close stomata.

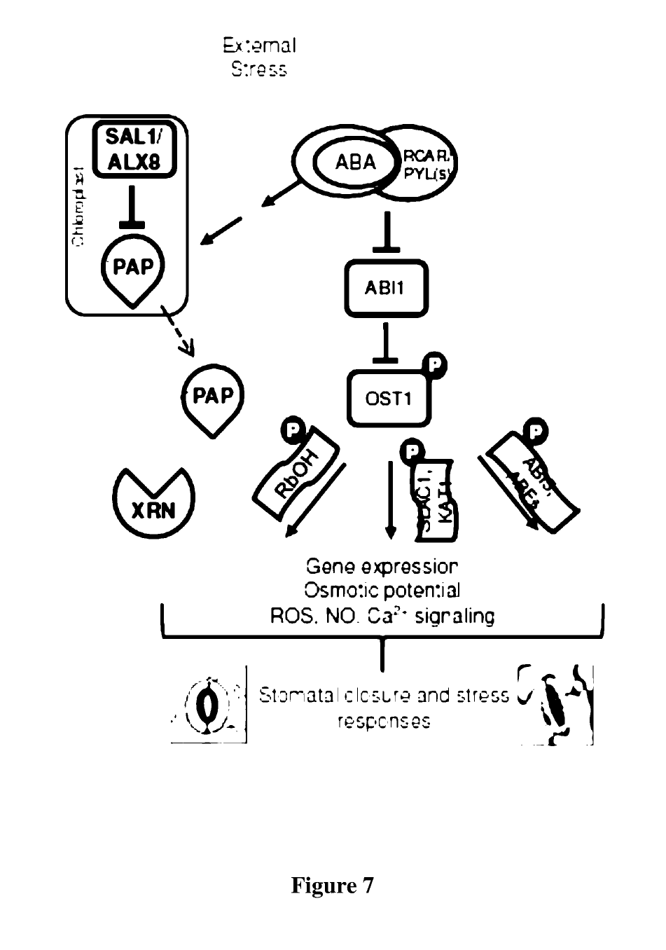

FIG. 7 shows a proposed model of ABA and PAP mediated stomata regulation based on our findings. In this model ABA binds to PYR/PYL/RCAR receptors and triggers stomatal closure primarily via ABA-receptor complexes that bind to protein phosphatase 2Cs such as ABI1, releasing their inhibition of SNF1-related protein kinase 2 (SnRK2) OST1 and promoting stomatal closure via phosphorylation of downstream proteins, examples of which are shown. ABI5 and ABFs represent transcription factors target of OST that regulate ABA-responsive genes; SLAC1 and KAT1 are anion and cation channels that mediate chloride efflux and potassium influx respectively, to modulate osmotic potential of the guard cells; and RboH is a NADPH oxidase that promotes the ROS burst. Thus, parallel and intersecting signaling networks mediated by ABI1/OST1, IP3, NO, Ca2+ and ROS all contribute to closure. Our prior work shows that part of the drought tolerance in alx8 is ABA independent. Our current studies indicate that ABA stimulates accumulation of PAP presumably via one of the ABA receptors. PAP levels are controlled in the chloroplast by SAL1/ALX8 and can move to the nucleus where PAP inhibits XRN2,3 altering RNA metabolism and changing gene expression. PAP is also able to induce the ROS burst and the activation of ion channels for stomatal closure. The ABA or drought mediated complementation of OST1 or ABI1 occurs in the absence of SAL1, XRN2,3 or when PAP is manipulated exogenously or endogenously. As discussed further below, this ABA-induced change in gene expression and stomatal closure cannot be a simple epistatic process, but is more likely to be a parallel pathway, although some common downstream processes are clearly involved.

### Example 4—sal1 Mutation, and Resulting Increase in PAP Concentration, Rescues ABA-Insensitive Stomatal Closure Mutants

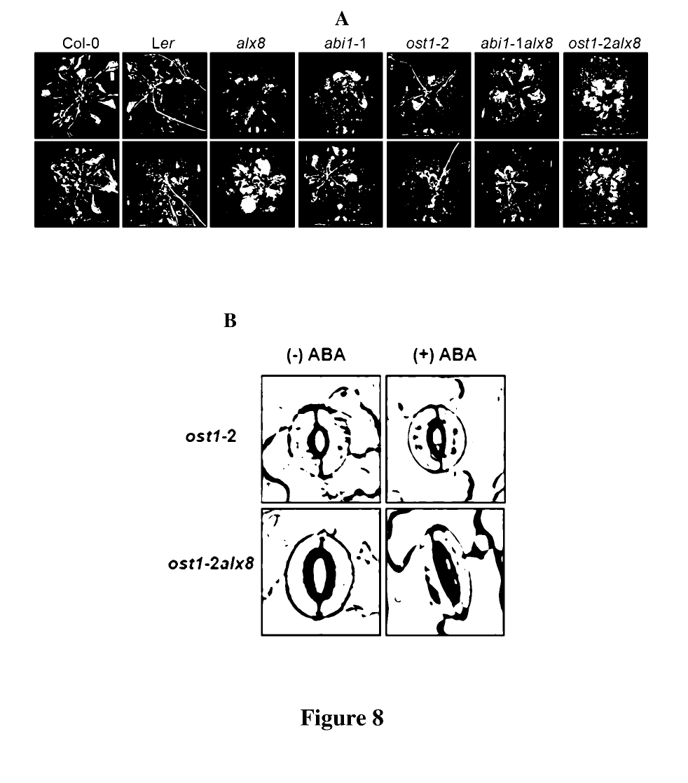

When challenged by drought stress abi1-1, aba2-3 and ost1-2 mutants (ABA-insensitive mutants which normally cannot close stomata) displayed widespread wilting and death before wild type. Conversely, all double and single mutants containing the alx8 lesion were green and photosynthetically-viable (FIG. 8A), maintained their relative water content, and displayed stomatal closure on ABA-stimulation (FIG. 8B). Differences in stomatal morphology or density as measured by CryoSEM do not provide a persuasive mechanism for the drought tolerance. Indeed, we calculated the net effect of stomatal number and aperture in well-watered plants to be a similar total stomatal opening area per leaf area. The complementation could not be readily explained by differences in ABA content, which were not significantly different in the genotypes examined, and, similarly, in aba2-3 alx8 the tolerance was similar to alx8, not aba2-3. Collectively, these results indicate that SAL1-PAP complementation functions in ABA signaling transduction, not synthesis. That is, elevated PAP in the double mutants should not be able to complement the drought sensitivity via increased ABA, as its effect should be blocked by abi1-1 or ost1-2.

Our results show that PAP could restore multiple aspects of ABA-stimulated stomatal closure in ABA insensitive genotypes including leaf temperature, stomatal conductance and aperture (FIG. 5). Stomatal aperture abi1-1 and ost1-2 plants did not respond to exogenous ABA. However, similar ABA-induced changes in ost1-2 alx8 were observed to that in wild type and alx8, indicating that stomatal responsiveness to the ABA treatment in ost1-2 was restored by the absence of SAL1, bypassing the blocked canonical ABI1/OST1 pathway. The restoration of ABA sensitivity in the double mutants is consistent with non-epistatic complementation: that is, if SAL1-PAP and OST1 act epistatically then ost1-2 alx8 stomata should not respond upon addition of ABA. The explanation most consistent with the observations is that the SAL1-PAP pathway bypasses the canonical OST1 and ABI1.

The aforementioned data raised the question as to whether PAP was stimulating an alternate ABA pathway and/or was in and of itself a regulator of stomatal movement. Stomatal closure in ost1-2, which was not responsive to exogenous ABA, responded to increased PAP levels in +LiCl and ABA+LiCl when sprayed on whole plants. K⁺, Cl⁻ and Ca²⁺ fluxes (which are key terminal outputs of ABA signaling that directly induce stomatal closure) in wild type, ost1-2, alx8 and ost1-2 alx8 plus/minus ABA were consistent with the restoration of ABA responsiveness in the double mutant. Of equal significance was that PAP alone mediated similar rapid changes in ion fluxes in all genotypes comparable to that of Ca²⁺ (and ABA in wild type); and in contrast to that for ost1-2 plus ABA. Therefore, PAP both restores ABA responsiveness in guard cells of abi1-1 and ost1-2, and, is in and of itself sufficient to mediate stomatal closure.

We investigated if PAP acts in guard cell signaling via two different secondary messengers, NO and ROS, that are required in the induction of ion fluxes necessary for stomatal closure. PAP likely mediates closure independent of NO as the presence of the inhibitor of NO synthase, N(G)-methyl-L-arginine (NMA) reduced ABA-induced stomatal closure in Col-0Ler, but did not affect ABA-induced closure in alx8, ost1-2 or ost1-2 alx8 plants. The ROS burst known to stimulate stomatal closure was observed in response to ABA treatment in wild type and was attenuated as expected in ost1-2. Significantly, the ROS burst was observed in the double mutant, to a similar degree to that of wild type. Of even greater significance was the observed phenocopying of the timing and extent of the ABA-induced ROS burst by exogenous PAP in wild type, ost1-2 and ost1-2 alx8. Thus, PAP stimulates ROS and stomatal closure independent of SnRKs and probably NO.

We then investigated if our recently reported epistatic relationship between XRNs and SAL1 in retrograde signaling could also function in guard cell signaling, as the XRNs are inhibited by PAP. The double mutant xrn2 xrn3 is drought tolerant as it genetically simulates PAP inhibition of nuclear XRNs. Indeed, ost1-2 xrn2 xrn3 plants closed their stomata in response to ABA to the same degree as wild type and alx8. Given that altering XRN-mediated RNA metabolism is sufficient to restore stomatal closure in ost1-2, the possibility that the restoration of ABA responsiveness in ost1-2 alx8 is transcriptionally mediated was investigated. Prior to undertaking arrays we trialed the application of the nuclear transcriptional inhibitor, actinomycin D (AD) which suppressed ABA-mediated closure to a lesser extent in ost1-2 alx8 than in the wild type.

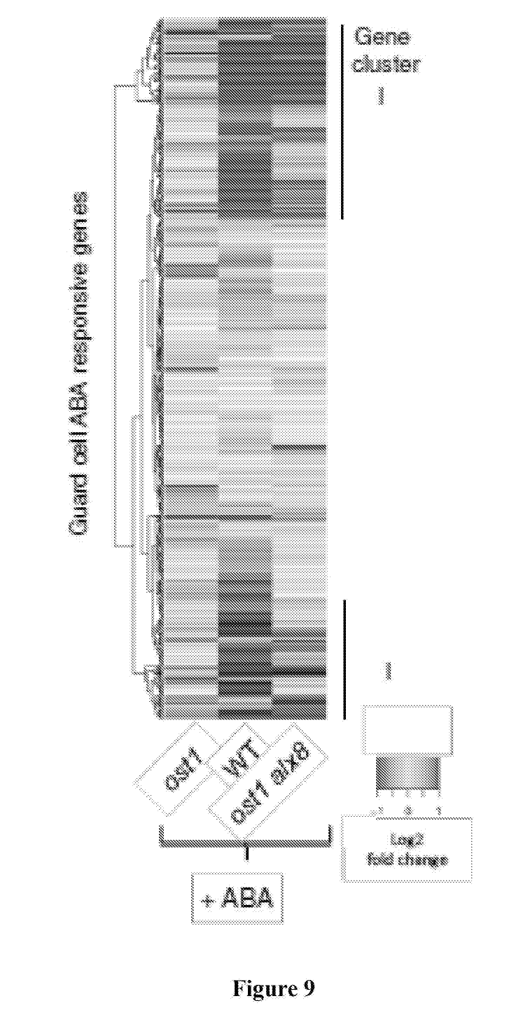

We performed transcriptome analyses of leaves treated with ABA and compared sets of genes that were differentially regulated among genotypes to investigate the molecular mechanism underlying restoration of ABA-responsiveness in ost1-2 alx8 (FIG. 9). First, it was found that the impaired ABA response in ost1-2 was substantially restored in ost1-2 alx8. That is, of the 1723 genes that respond differently to ABA in the wild type compared to ost1-2, in the double mutant plus ABA the induction of 1705 (99%) was restored, such that they were not significantly different to wild type plus ABA. The response was attenuated and there were other significant transcriptional differences between genotypes and treatments. Second, hierarchical clustering of the expression of 1173 ABA-responsive genes known to be expressed in guard cells in the four genotypes also showed that a large proportion of the genes induced by ABA in wild type leaves were not induced in ost1-2, but were significantly increased in ost1-2 alx8 leaves (gene cluster I, FIG. 9).

To investigate whether there were marked changes in the transcription of proteins that might account for the restoration of ABA responsiveness we analyzed the expression of genes in ABA signaling. Hierarchical clustering revealed a few subsets that were differentially expressed in all alx8 backgrounds and treatments compared to wild type+ABA and a few genes that were more highly expressed in alx8 and ost1-2 alx8 in response to ABA, but not in ost1-2 (gene cluster II). For the seven ABA receptors and three SnRK2 kinases (including OST1) detected as expressed there were no changes in their expression consistent with the restoration of ABA responsiveness in the double mutant, in agreement with PAP closing stomata independently of SnRKs. Some changes in expression of the PP2Cs in different genotypes and treatments were consistent with the complementation (gene cluster III).

Significantly, several genes involved in diverse, interlinked aspects of ABA signaling were differentially expressed in ost1-2 alx8. This included 10 transcription factors, 18 calcium signaling proteins [Calcium-Dependent Protein Kinases (CDPKs), Calcineurin B-like (CBLs) and CBL-Interacting Protein Kinases (CIPKs)], and genes directly regulating ROS homeostasis as well as protein import (table S11); all of which are critical in regulating the activities of inward rectifying channels (KATs) and slow anion channels (SLAC1/SLAHs), to mediate stomatal closure. Hence, the transcriptome data shows a degree of correlation with ROS, ion fluxes and stomatal closure in ost1-2 in response to ABA when PAP levels are modulated (FIG. 8B). It is likely that instead of changed expression of specific signaling components, the global transcriptional reprogramming of various aspects of the ABA network by PAP is sufficient to cumulatively restore stomatal closure in ost1-2 alx8.

The restoration of ABA sensitivity by PAP via transcription is also supported by the following observations. ABA-induced rapid stomatal closure (<10 min; FIGS. 5B and 5D) indicated that activation of the ABA signal transduction network, including transcriptional changes, occurs very quickly. The complete initiation of nuclear transcription in response to chloroplast signals is also extremely fast, in some cases within 1 min of signal initiation. This indicates that the chloroplast signal PAP is able to rapidly induce the necessary transcriptional reprogramming required for induction of ABA signaling. Indeed, the stomatal closure kinetics in PAP-treated epidermal peel is nearly identical to that of ABA (FIGS. 5B to 5D). Therefore, our current data unequivocally demonstrate that the SAL1-PAP-XRN retrograde pathway regulates multiple aspects of the ABA network, independent of OST1 and the SnRKs, but via ROS and changes in mRNA abundance.

### Example 5—Oxidative Redox Balance Favours Reduced SAL1 Activity and PAP Accumulation

The phosphonucleotide 3′-phosphoadenosine 5′-phosphate (PAP), is a by-product of sulfation reactions in secondary metabolism and intracellular signaling. The catabolic phosphatases (such as the Arabidopsis thaliana SAL1) and the encoding nucleotide sequences are highly conserved in the plant kingdom (monocots and dicots; see FIGS. 10-12). PAP regulates multiple metabolic pathways including sulfur and glutathione homeostasis as well as non-sulfation processes associated with photosynthetic signaling, salt toxicity and liver pathologies that can cause death. In Arabidopsis thaliana, PAP is degraded by Arabidopsis SAL1 (AtSAL1; the full encoded protein having the amino acid sequence as shown in FIG. 13—SEQ ID NO: 1, the encoding genomic sequence being shown in FIG. 14—SEQ ID NO:2) in chloroplasts, and acts as a retrograde signal, as described above, and in Example 7: it accumulates during drought and high-light stress and regulates >700 stress-inducible nuclear genes, including Ascorbate Peroxidase 2, a cytosolic reactive oxygen species (ROS) scavenger sensitive to hydrogen peroxide (H₂O₂) and photosynthetic redox state in chloroplasts. Yet, like virtually all chloroplast retrograde signaling pathways, it is unknown how stress is initially sensed by this pathway.

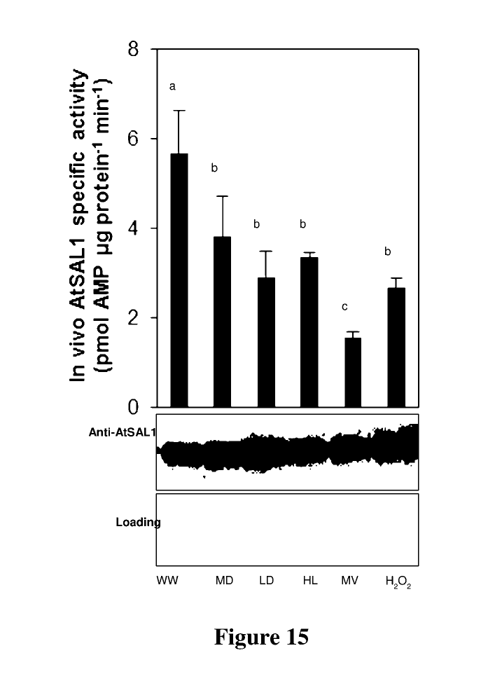

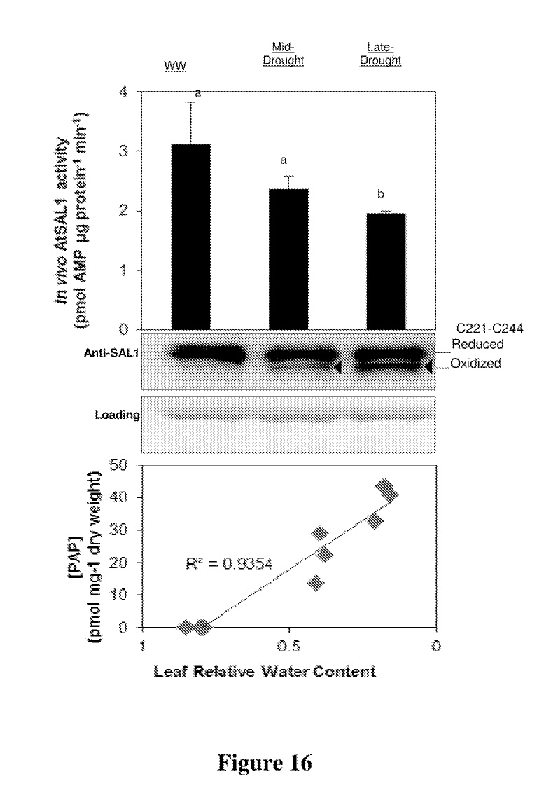

Here, we investigated whether accumulation of PAP as a signaling molecule is directly modulated by chloroplastic redox poise and oxidative stress. If AtSAL1 is the primary regulator of PAP, then drought, high-light, H₂O₂ and superoxide (O₂⁻) treatments might be expected to alter AtSAL1 protein levels, but they did not: instead, they significantly lowered AtSAL1 specific activity via another mechanism (FIGS. 15 and 16). We genetically manipulated chloroplast redox homeostasis; disabling key components of the Photosystem I (PSI) water-water cycle, ascorbate detoxification of ROS, and the glutathione cellular redox buffer. All of these changes exacerbated the decrease in AtSAL1 activity in response to high-light induced oxidative stress. Furthermore, in vitro AtSAL1 activity can be titrated by redox state, with a redox midpoint potential (Eₘ) within the cellular physiological range (FIG. 17). These results indicate that the chloroplast redox state directly contributes to the generation of the retrograde signal PAP by modulating AtSAL1 activity.

To understand the basis of AtSAL1 redox regulation, we crystallized AtSAL1 (PDB 4Y7Q) and identified three cysteine residues (C173, C221, C244; FIG. 18; C119, C167 and C190 in the mature peptide) as potential targets for redox regulation. AtSAL1 contains an intramolecular C221-C244 disulfide pair located on opposite strands of a beta-sheet (FIG. 18) that can form a forbidden disulfide: a metastable switch often used to control protein activity. Indeed, under oxidizing conditions, decreased AtSAL1 activity is accompanied by formation of a C221-C244 disulfide bond (FIG. 19; C221 shown as C167, and C244 shown as C190) reversible when conditions are returned to a reducing state (FIGS. 19B and D). Mutagenesis of cysteine residues in AtSAL1 to alanine abrogated disulfide formation and made the activity of the enzyme insensitive to the redox state (FIGS. 19B and D). Significantly, the C221-C244 disulfide also forms in vivo in drought-stressed Arabidopsis (FIGS. 16 and 19A), in correlation with decreased AtSAL1 activity (FIG. 17A) and PAP accumulation. Hence, the C221-C244 disulfide is a key post-translational mechanism regulating AtSAL1 activity.

Although previously reported as a monomer, we found that AtSAL1 exists in a monomer-dimer equilibrium (FIG. 20B). The crystal structure reveals a dimer interface, identified visually and with the Protein Interfaces, Surfaces and Assemblies (PISA) server, that is centered on C119 (FIG. 17; C119 shown as C173), suggesting a role for an intermolecular disulfide formation in dimerization. Our previous analysis of AtSAL1 activity (FIG. 15) was performed on a mixture of dimer and monomer. We observed strikingly different behavior of the monomeric and dimeric species if they are separated: whereas the monomer has high activity and is resistant to oxidation and inactivation, the dimer is sensitive to oxidation and is rapidly inactivated in sufficiently oxidizing conditions (FIG. 20A). Additionally, the dimer is stable under oxidizing conditions, owing to the presence of an intermolecular disulfide, but under sufficiently reducing conditions the protein returns to equilibrium between monomer and dimer. The same trend is observed in in-vivo samples, where the proportion of dimeric AtSAL1 increases under stress conditions. Thus, dimerization is required to achieve inactivation, and the monomer-dimer equilibrium itself can be further shifted in favor of the dimer under oxidizing conditions through the formation of a second, intermolecular disulfide bridge.

Given that dimerization appears to be essential for C221-C244 disulfide bond formation, we sought to investigate the mechanism that underlies this process. When we performed molecular dynamics (MD) simulations of monomeric AtSAL1 and estimated the flexibility of residues along the chain from the root mean square fluctuation (RMSF) of the C_{α} atoms, the simulation of reduced AtSAL1 shows an increase in the distance between C221 and C244 sulfur atoms from 5.9 Å observed in the non-covalent dimer of the crystal structure, to 6.9±0.8 Å (mean±standard deviation). This suggests that formation of C221-C244 is unlikely to be spontaneous in the monomer. When simulations were carried out with the C221-C244 disulfide, we observed a significant reduction in the flexibility of the dimerization interface (FIG. 21); in particular the RMSF for the C_{α} of C119 decreased from 2.4 to 1.4 Å. This highlights the interconnectedness of these regions of the protein and the importance of molecular flexibility. We propose that a positive feedback loop exists, where stabilization of the dimer interface leads to formation of the Cys221-Cys244 disulfide, which in turn further stabilizes the dimer. This also suggests the basis for the loss of activity: although no direct change to the active site of the enzyme is observed upon formation of either disulfide bond, enzyme inhibition through the stabilization of an inactive conformation is a well-established mechanism.

The redox buffer glutathione regulates chloroplast retrograde signaling and protein activity, and cysteine glutathionylation by the redox buffer glutathione can enable intramolecular disulfide bonding with a proximal cysteine via thiol/disulfide exchange. We thus investigated whether regulation of AtSAL1 by glutathionylation could bypass the requirement for a priori protein dimerization, and found that glutathionylation down-regulated both monomer and dimer enzymatic activities. The decreased activity in AtSAL1 oxidized with oxidized glutathione (GSSG) correlated with the formation of cysteine-glutathione disulfides in redox-sensitive C173 and C244 as detected by mass-spectrometry. The GSSG-treated AtSAL1 is able to form the C221-C244 disulfide, probably via thiol-disulfide exchange.

Therefore, PAP accumulation could be triggered by inhibiting AtSAL1 enzymatic activity via two redox pathways: the first involving dimerization and intermolecular disulfide bonding followed by the formation of an internal disulfide, and the second involving glutathionylation followed by the internal disulfide. Both pathways decrease enzyme flexibility via the internal disulfide (FIG. 21) and collectively inhibit AtSAL1 activity.

Many moonlighting proteins are evolutionarily-ubiquitous enzymes that have been implicated in diverse processes including metabolism and disease. Yet, no moonlighting stress sensors have been identified to date. The redox regulation of AtSAL1 by multiple processes (FIGS. 15, 16, 17, 19D and 20) suggests a moonlighting role as a direct and reversible sensor of oxidative stress within plant energy organelles, which is most likely conserved in the plant kingdom, despite its primary and evolutionarily-ubiquitous role in the metabolic control of sulfation. We therefore investigated whether similar moonlighting functions and mechanism of regulation are also present in AtSAL1 homologs in O. sativa (OsSAL1), S. cerevisiae (ScHAL2) and H. sapiens (HsBPNT1). The human gene is of particular interest as duplication has resulted in one copy, GPAPP, functioning in sulfation reactions, whereas the role of the second copy, BPNT1, is enigmatic. BPNT1 mouse knockouts do not show any phenotype related to sulfur assimilation, but they do present a 50-fold increase in PAP and severe liver pathologies, including hepatocellular damage leading to hypoproteinemia, edema and premature death.

Human HsBPNT1, yeast ScHAL2 and rice OsSAL1 all exhibited redox-sensitive activity correlating with glutathionylation of the protein (FIG. 17 shows results for the rice protein, also shown in FIG. 18 showing the surface cysteine residues—C203 and C221) equivalent to C221 and C244 in AtSAL1). In agreement with this, surface-exposed cysteines were observed in the crystal structures of HsBPNT1 (PDB ID: 2WEF) and ScHAL2 (1KA1), and in homology modeling of OsSAL1 based on the 1KA1 structure (38.2% identity). Yeast cells accumulated more PAP upon oxidative stress by O₂⁻ generator methyl viologen (MV) treatment, similar to oxidatively-stressed Arabidopsis and SAL knockouts in plants and mice. PAP inhibits RNA processing exoribonucleases (XRNs) resulting in co-expression of transcriptomes in sal1 and xrn Arabidopsis mutants. We observed co-expression between Δxrn1 and oxidatively-stressed yeast cells and significant enrichment of stress-response genes in the 2,666 transcripts targeted by XRN1 in human cells, with a significant proportion being expressed in multiple oxidation experiments. Thus, similar to Arabidopsis, the conserved redox regulation of ScHAL2 and HsBPNT1 allows activation of PAP-XRN stress signaling.

Intriguingly, although the moonlighting function of redox sensing is conserved, some aspects of ScHAL2 and HsBPNT1 regulation are not. ScHAL2 activity could be inhibited by drought stress when heterologously-expressed in Arabidopsis, indicating it is sensitive to cellular redox state. Yet, ScHAL2 lacks the three cysteines at positions structurally equivalent to those of AtSAL1, including the C221-C244 intramolecular disulfide. Therefore we introduced three additional cysteines (T21C, F127C, Y196C) into ScHAL2 (ScHAL2+3C), so that Y196C introduces potential for an intramolecular disulfide similar to AtSAL1. ScHAL2+3C was more redox sensitive in vivo than the wild-type form: yeast Δhal2 overexpressing ScHAL2+3C significantly accumulated PAP when challenged with mild H₂O₂ stress, whereas those overexpressing wild-type ScHAL2 did not, despite no difference in enzyme activity. This shows that the mechanism of AtSAL1 inactivation can be engineered into related proteins, and suggests that there is a more complex mechanism of regulation in Arabidopsis compared to Saccharomyces. Indeed, while the enzymes share function and redox-sensitivity across organisms, AtSAL1 appears more responsive to ROS and redox state. Differences in organellar localization (chloroplasts and mitochondria in Arabidopsis versus cytosol in humans and yeast) could be expected to have driven these differences, but targeting of AtSAL1 exclusively to the cytosol, chloroplast, nucleus and mitochondria could restore PAP levels in Arabidopsis Δsal1 to wild type (WT) and in each instance AtSAL1 activity was inhibited during drought. Thus, the redox regulation of SAL1 homologs could arise in evolution independent of the constraints of subcellular location.

Transient elevation of PAP levels as an oxidative stress signal by coupling its accumulation to the redox state perceived by SAL1/HAL2/BPNT1 appears to be a key process. In plants, plastid redox couples and ROS, particularly those associated with Photosystem I, are implicated in organelle signaling. Oxidative modulation of the AtSAL1-PAP pathway may provide a hub for perception of short-lived ROS that alter nuclear gene expression, but paradoxically cannot leave the organelles. Thus, AtSAL1 redox regulation by multiple posttranslational cysteine modifications may provide a potential point of convergence and capacity for fine-tuning these redox cues. The responsiveness of AtSAL1 to GSH/GSSG, the conservation of redox regulation across three eukaryotic kingdoms, and the observation that the AtSAL1 redox midpoint potential (FIGS. 17B and 22B) is similar to other sulfur and glutathione enzymes suggests redox control of SAL1 could have arisen primarily for metabolic homeostasis, but has taken on stress signaling functions.

The redox sensitivity of SAL1 also provides a mechanism for elevating PAP levels in plant in situ by, for example, treating the plant(s) with substances that induce oxidative conditions and/or applying artificial stress conditions to plants. Alternatively, or in addition, plants could be engineered to include cysteines at desired positions to make them more sensitive to oxidative conditions, making it easier to induce reduced SAL1 activity and allow accumulation of PAP.

### Example 6—SAL and its Encoding Sequence are Highly Conserved in the Plant Kingdom

SAL1 amino acid (FIG. 13; cysteine residues highlighted) and homologue sequences from various plant species were retrieved from the Phytozome database and aligned by ClustalW and Gblocks. The results are shown in FIGS. 10 (dicots) and 11 (monocots). Similarly, SAL1 (FIG. 14; start and stop codons, as well as cysteine-encoding codons highlighted) and homologue cDNA sequences have been aligned, and conserved cysteine-encoding codons highlighted (see FIG. 12).

Notably, all of the sequences show a high degree of conservation and, although the first cysteine residue (Cys 173) appears to be shared amongst Brassica members only (see FIG. 10, as well as the encoding sequence alignment: FIG. 12), Sal1 cysteine residues 221 and 244 are fully conserved across all dicots (see FIG. 10) and monocots (see FIG. 11), and the encoding codons are also substantially conserved across all species (allowing for a shift between grasses and the other plants investigated (see FIG. 12).

### Example 7—PAP Suppresses Seed Germination

We also investigated if PAP could also interact with ABA signaling in seeds. We found that PAP content in wild type seeds was seven fold higher than in leaves and this correlated with lower levels of the SAL1 transcript and SAL1 protein (FIG. 24C). Imbibition of dry seed was shown to increase SAL1 expression by more than 10 fold, which is the most dramatic change in SAL1 expression in 3,000 different perturbations (FIG. 24A). Indeed, the up-regulation of SAL1 is part of the transcriptional signature that defines germinating seeds. These findings indicate that PAP content may be specifically elevated in dry wild type seed to regulate germination. Indeed, higher levels of PAP in sal1 seed delayed its germination compared to wild type (FIG. 24B). More significantly, we were able to demonstrate that exogenous PAP can be perceived and repress germination in wild type seeds in a dose-dependent manner in conjunction with exogenous ABA (FIG. 24B) or lower levels of GA. Similarly, the lack of germination inhibition of abi1-1 in response to ABA, or ABA and the GA biosynthesis inhibitor, paclobutrazol, was largely restored in abi1-1 alx8 mutants under both treatments. The addition of PAP alone also restored ABA responsiveness in abi1-1. Both genetic and exogenous manipulation of PAP inhibits germination and enhances ABA sensitivity in Arabidopsis seeds; thus, PAP also participates in ABA-mediated germination control in seeds in an ABI1-independent manner, analogous to its role in guard cells.

PAP fulfills many of the criteria for secondary messengers. Its synthesis, catabolism and site of action are compartmentalized. We have shown that ABA stimulates PAP accumulation and modulation of any aspect of the PAP pathway (signal abundance, degradation, transport, localization, and perception) can impact on processes mediated by ABA. Additionally, PAP induces specific responses in different plant organs when levels are temporally manipulated exogenously or endogenously. Exogenous PAP can also move within organs to exert its effect in a different cell type, such as from the petiole via the vasculature to guard cells to induce stomatal closure, and across the seed coat to control germination of the embryo within. That both PAP and SAL1 are regulated in dry and germinating seed indicates it has physiological effects in different cell types. Additionally, foliar PAP accumulation is not only specifically regulated and induced in response to environmental stimuli such as drought, but has defined downstream molecular and physiological outputs: high light and drought triggering transcriptional changes, ROS bursts and ion fluxes in guard cells in an OST1-independent pathway and in an XRN-mediated manner. Finally, while all of the aforementioned features of PAP are reminiscent of well-established secondary messengers, we reason that an important test would be its conservation of function beyond Arabidopsis to other higher plants. Indeed, we present evidence that exogenous PAP regulates stomatal conductance in barley, a monocot (FIG. 6D).

Therefore, we conclude that PAP, and presumably analogues thereof, functions as a secondary messenger that acts in multiple cells, tissues and species, providing a previously unanticipated additional level of input and control into ABA-mediated signaling (FIG. 5). The interaction between PAP and ABA signaling indicates that input from chloroplasts, in response to stimuli such as oxidative stress, can be incorporated into other cellular responses to drought.

### Example 8—PAP Analogues Elicit Similar Stomatal Closure to PAP

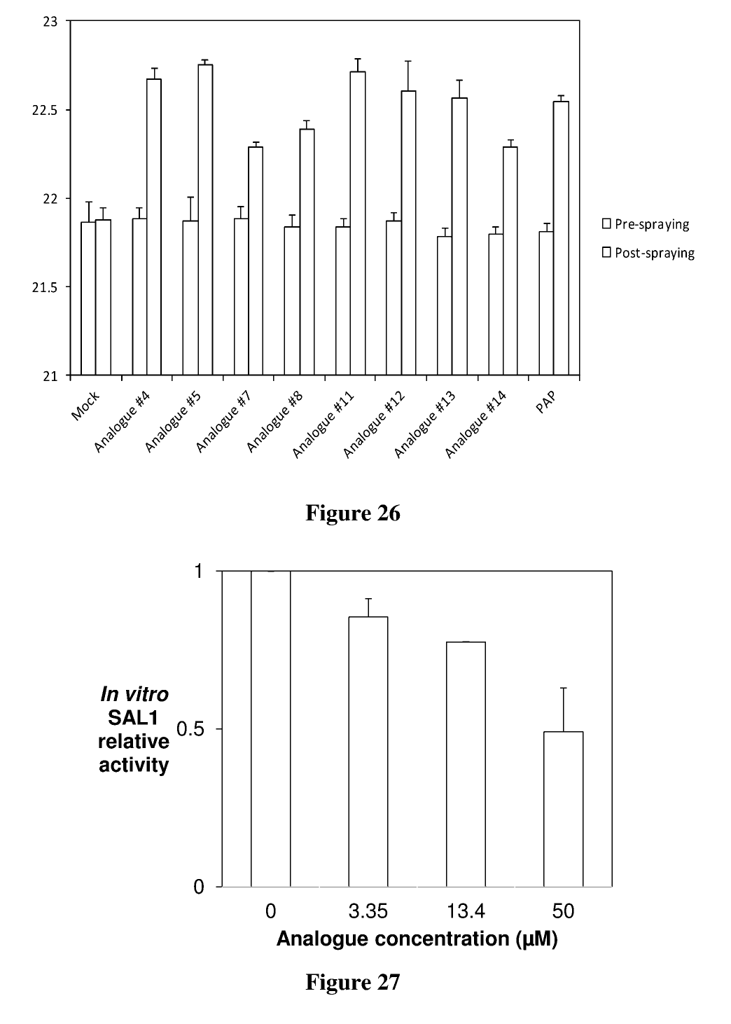

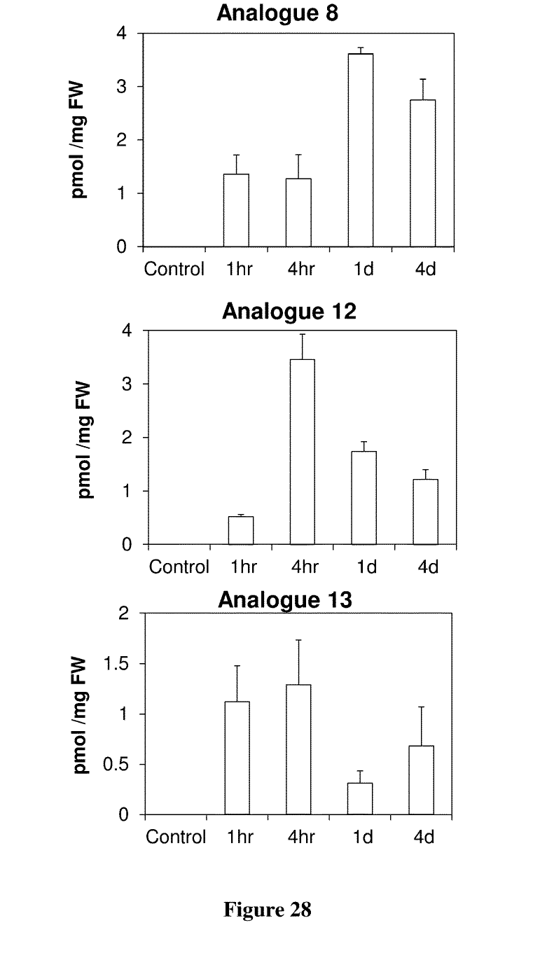

Treatment of leaf peels with cordycepin (10 μM) elicited rapid stomatal closure similar to the PAP response (FIG. 25), but at one tenth the concentration (PAP at 100 μM). Furthermore, other PAP analogues selected from those identified in Table 1 were also found to elicit stomatal closure in Arabidopsis leaves (FIG. 26), and analogue 12 was found to inhibit in vitro SAL1 activity (FIG. 27). These results validate our postulation that PAP analogues (and suitable derivatives) will act in the same manner as PAP, and provide an alternative manner of eliciting stress tolerance, especially drought tolerance, high light tolerance, or both drought and high light tolerance, in plants. Similarly, it is contemplated that PAP analogues will inhibit seed germination, providing a means for controlling germination in seeds.

### Example 9—PAP Analogues Accumulate in Arabidopsis Leaves after Foliar Spray

Soil-grown wild-type Arabidopsis thaliana plants sprayed with analogue 8, analogue 12 or analogue 13 (as identified in Table 1) showed rapid accumulation of PAP analogues to levels comparable with those for PAP found in leaf tissue in vivo, shortly after spraying, confirming the feasibility of mimicking PAP-induced events or inhibiting 3′(2′),5′-bisphosphate nucleotidase activity in plants in vivo by spraying plants with PAP analogues or derivatives.

It will be appreciated that, although specific embodiments of the invention have been described herein for the purpose of illustration, various modifications may be made without deviating from the spirit and scope of the invention as defined in the following claims.

1. A method for increasing stress tolerance of a plant, said method comprising increasing the levels of 3′-phosphoadenosine-5′-phosphate (PAP) or a derivative or analogue thereof in cells of said plant compared to an untreated or wild-type plant grown under the same stress conditions.

2. The method of claim 1, wherein the levels of PAP or said derivative or analogue thereof are increased in the nucleus of said cells.

3. The method of claim 1 or claim 2 which comprises administering to said plant PAP or said derivative or analogue thereof.

4. The method of claim 1 or claim 2 which comprises administering to said plant a substance that:
(i) enhances or promotes synthesis or accumulation of PAP or said derivative or analogue thereof; or
(ii) inhibits or compromises an activity metabolising or removing PAP or said derivative or analogue thereof.

5. The method of claim 4 which comprises administering to said plant a substance that inhibits or reduces the activity of a 3′(2′),5′-bisphosphate nucleotidase, optionally a SAL protein in said cells.

6. The method of claim 5, wherein said substance is a 3′(2′),5′-bisphosphate nucleotidase activity inhibitor.

7. The method of claim 6, wherein said substance is selected from PAP analogues or derivatives, or lithium or sodium ions.

8. The method of claim 5, wherein said 3′(2′),5′-bisphosphate nucleotidase is a SAL protein and said substance induces:
(i) at least partial inactivation of a SAL protein;
(ii) oxidative conditions inside said cells;
(iii) conformational changes, polymerization, denaturation, degradation, or any combination thereof of a SAL protein;
(iv) reactive oxygen species inside said cells; or
(v) any combination of (i) to (iv).

9. The method of claim 8, wherein one or more mutations are introduced into an endogenous SAL-encoding nucleotide sequence which makes the encoded SAL protein more susceptible to conformational changes, polymerization, denaturation, or degradation, or any combination thereof.

10. The method of claim 8, wherein an endogenous SAL-encoding sequence in said cells is replaced by an exogenous SAL-encoding sequence that encodes a SAL protein which is more susceptible to conformational changes, polymerization, denaturation, or degradation, or any combination thereof.

11. The method of claim 9 or claim 10 wherein the SAL protein which is more susceptible to conformational changes, polymerization, denaturation, or degradation, or any combination thereof comprises cysteine residues at positions equivalent to one or more of positions 173, 221 and 244 of SEQ ID NO:1.

12. The method of any one of claims 1 to 11 wherein said administration is foliar administration.

13. The method of any one of claims 1 to 11 wherein said administration is effected by application to the soil.

14. The method of claim 1 or claim 2, which comprises:
(i) introducing a mutation in an endogenous nucleotide sequence;
(ii) replacing an endogenous nucleotide sequence with an exogenous nucleotide sequence; or
(iii) inserting an exogenous nucleotide sequence;
in cells of said plant, wherein the resulting reduced activity, replaced activity or increased activity in said cells associated with said mutation or exogenous nucleotide sequence results in increased accumulation of PAP or said derivative or analogue thereof under at least stress conditions compared to a wild-type plant.

15. The method of claim 14 which comprises:
(i) introducing a mutation in an endogenous 3′(2′),5′-bisphosphate nucleotidase activity-encoding sequence, optionally a SAL-encoding sequence; or
(ii) replacing an endogenous 3′(2′),5′-bisphosphate nucleotidase-encoding sequence, optionally an endogenous SAL-encoding sequence, with an exogenous 3′(2′),5′-bisphosphate nucleotidase-encoding sequence, optionally an exogenous SAL-encoding sequence;
into said cells, whereby the 3′(2′),5′-bisphosphate nucleotidase activity, optionally SAL activity, in said cells is at least reduced under at least stress conditions compared to a wild-type plant.

16. The method of claim 15, wherein the 3′(2′),5′-bisphosphate nucleotidase is a SAL protein, and said mutated endogenous SAL-encoding sequence or exogenous SAL-encoding sequence encode a SAL protein comprising cysteine residues at positions equivalent to one or more of positions 173, 221 and 244 of SEQ ID NO:1.

17. The method of claim 16, which further comprises administering to said plant a substance that induces increased oxidative conditions inside said cells.

18. A method according to any one of claims 1 to 17, wherein said plant:
(i) yields a greater amount of above-ground plant matter;
(ii) grows faster;
(iii) develops faster;
(iv) survives longer;
(v) is photosynthetically viable for longer;
(vi) retains more water in leaves; or
(vii) prevents loss of above-ground matter compared to untreated conditions;
compared to an untreated or wild-type plant grown under the same stress conditions.

19. A method according to any one of claims 1 to 18, wherein said conditions are abiotic stress conditions.

20. The method according to claim 19, wherein said stress conditions are selected from the group comprising increased sodium, increased salinity, drought, light stress, and pH stress.

21. A method for extending the dormancy of a seed, as compared to an untreated seed, said method comprising accumulating in cells within said seed an increased level of PAP or a derivative or analogue thereof compared to an untreated or wild-type seed.

22. The method of claim 21, wherein said seed is obtained from a plant obtained by a method according to any one of claims 1 to 20.
